(19) United States
(12) Patent Application Publication
(10) Pub. No.: US 2008/0241184 A1
(43) Pub. Date: Oct. 2, 2008

(54) CANINE INFLUENZA VACCINES

(76) Inventors: Jules Maarten Minke, Corbas (FR); Kemal Karaca, Athens, GA (US); Jiansheng Yao, North York (CA)

Correspondence Address: Judy Jarecki-Black, Ph.D, J.D.;c/o Merial Limited, 3239 Satellite Blvd., Duluth, GA, 30096-4640, US

(21) Appl. No.: 12/020,656

(22) Filed: Jan. 28, 2008

Related U.S. Application Data
(63) Continuation-in-part of application No. 11/264,622, filed on Nov. 1, 2005.
Application No. 11/264,622 is a continuation-in-part of application No. 11/211,983, filed on Aug. 25, 2005, now Pat. No. 7,384,642.

(51) Int. Cl. A61K 39/00; A61P 37/00

(52) U.S. Cl. 424/199.1; 424/209.1

(57) ABSTRACT

The present invention encompasses influenza vaccines, in particular canine influenza vaccines. The vaccine may be a recombinant poxvirus vaccine or an inactivated vaccine. The invention also encompasses recombinant poxvirus vectors encoding and expressing influenza antigens, epitopes or immunogens which can be used to protect animals, in particular dogs, against influenza.

### INCORPORATION BY REFERENCE

This application is a continuation-in-part application of U.S. patent application Ser. No. 11/264,622 filed Nov. 1, 2005, which is a continuation-in-part of U.S. patent application Ser. No. 11/211,983 filed Aug. 25, 2005, which are both herein incorporated by reference.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention encompasses influenza vaccines, in particular canine influenza vaccines. The vaccine may be a recombinant poxvirus vaccine or an inactivated vaccine.

### BACKGROUND OF THE INVENTION

Respiratory diseases resembling influenza have infected thousands of dogs in the U.S. Recurrent outbreaks of severe respiratory disease characterized by coughing and fever have occurred in greyhounds at racing kennels. Pathological findings included severe pulmonary and plural hemorrhage, accurate to subacute erosive to hyperplastic tracheitis, bronchitis and bronchiolitis and bronchopneumonia. In 2004, eight greyhounds in Jacksonville, Fla. were killed by an equine influenza virus that jumped the species barrier from horses to dogs.

Equine influenza is a disease of horses, and the virus is in the same group of viruses that cause flu in people. The disease is present in horse populations throughout Europe, North America and parts of Asia, with horses typically developing a fever and a dry hacking cough. In the early stages of the disease, horses are reluctant to eat or drink for several days, but usually recover in two to three weeks.

H3N8 subtypes of equine influenza were previously isolated from lungs of two dogs from Florida and one dog from Texas who had died from the infection. Genetic sequence analyses and phylogenetic comparisons determined that all three canine isolates were closely related and evolved from contemporary strains of equine influenza H3N8. Immunohistochemistry demonstrated influenza antigen in bronchial gland epithelial cells, bronchial and bronchiolar epithelial cells and in alveolar macrophages. Seroconversion to canine influenza virus was demonstrated by hemagglutination inhibition and microneutralization assays.

Molecular and antigenic analyses of three influenza viruses isolated from outbreaks of severe respiratory disease in racing greyhounds revealed that they are closely related to H3N8 equine influenza virus (see, e.g., Crawford et al., Science. 2005 Oct. 21; 310(5747):482-5. Epub 2005 Sep. 26). Phylogenetic analysis indicated that the canine influenza virus genomes form a monophyletic group, consistent with a single interspecies virus transfer. Molecular changes in the hemagglutinin suggested adaptive evolution in the new host. The etiologic role of this virus in respiratory disease was supported by the temporal association of rising antibody titers with disease and by experimental inoculation studies. The geographic expansion of the infection and its persistence for several years indicate efficient transmission of canine influenza virus among greyhounds. Evidence of infection in pet dogs suggests that this infection may also become enzootic in this population.

In 2005, a mutated form of the Bird Flu (H3N8 Virus) was reported to have killed greyhounds in Massachusetts.

Accordingly, there is a need for an effective vaccine against influenza in canines.

Citation or identification of any document in this application is not an admission that such document is available as prior art to the present invention.

### SUMMARY OF THE INVENTION

The present invention encompasses canine influenza vaccines, which may be a recombinant canine influenza vaccine or an inactivated canine influenza vaccine.

In an embodiment wherein the canine influenza vaccine is a recombinant vaccine, advantageously, the vector is an avipox expression vector which may comprise a polynucleotide encoding an influenza antigen, epitope or immunogen. The influenza antigen, epitope or immunogen may be a hemagglutinin, matrix protein, neuraminidase, nonstructural protein, nucleoprotein, polymerase or any fragment thereof.

In an advantageous embodiment, the canine influenza antigen, epitope or immunogen is derived from a canine infected with influenza. For example, but not by limitation, influenza virus may be isolated from the broncho alveolar lavage and/or lung tissues of an affected dog. Isolation and characterization of the nucleotide sequence of the influenza virus infecting the dog may be done by routine experimentation by a person of ordinary skill in the art.

The canine influenza antigen, epitope or immunogen may be isolated from an equine influenza. The equine influenza may be an Ohio equine influenza, Kentucky equine influenza or a Newmarket equine influenza.

The avipox expression vector may be an attenuated avipox expression vector. In one embodiment, the avipox expression vector may be a canarypox vector, advantageously ALVAC. The influenza antigen, epitope or immunogen may be a hemagglutinin, such as H3. The canarypox vector may be CP 2242, CP1529 or CP1533 or expressing the H3 of a canine influenza isolate vCP2328)

The present invention also encompasses an inactivated influenza vaccine. The inactivated influenza vaccine may be an inactivated canine influenza. In another embodiment, the inactivated influenza vaccine may be an equine influenza, advantageously an Ohio equine influenza, Kentucky equine influenza or a Newmarket equine influenza. The vaccine may be inactivated with formalin or beta-propiolactone.

The invention also relates to method of eliciting an immune response against influenza in a canine, which may comprise administering a formulation comprising any one of the above recombinant influenza vaccine or inactivated vaccine and a pharmaceutically or veterinarily acceptable carrier, excipient or vehicle in an effective amount for eliciting an immune response. In an advantageous embodiment, an adjuvant may be added. The adjuvant may be aluminum hydroxide, aluminum phosphate, a carbomer or an oil-water-emulsion and optionally may comprise CpG. Advantageously, the administration may be subcutaneous or intramuscular.

The invention further relates to method of inducing an immune response against influenza in a canine, which may comprise administering a formulation comprising any one of the above recombinant influenza vaccine or inactivated vaccine and a pharmaceutically or veterinarily acceptable carrier, excipient or vehicle in an effective amount for inducing an immune response. In an advantageous embodiment, an adjuvant may be added. The adjuvant may be aluminum hydroxide, aluminum phosphate, a carbomer or an oil-water-emulsion, immunostimulating complex (ISCOM), and optionally may comprise CpG. Advantageously, the administration may be subcutaneous or intramuscular.

The invention further encompasses a kit for performing a method of eliciting or inducing an immune response which may comprise any one of the recombinant influenza vaccines or inactivated vaccines and instructions for performing the method.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the sequence of the insert in PJT004 (SEQ ID NO: 1);

FIG. 2 illustrates the sequence of the insert in PJT005 (SEQ ID NO: 2);

FIG. 3 illustrates a comparison of the amino acid sequence of EIV Ohio 03 strain HA to that of New Market strain H3 HA (SEQ ID NOS: 3 and 4);

FIG. 4A illustrates a construction of an ALVAC donor plasmid for generation of an ALVAC recombinant expressing codon optimized EIV H3 HA (Ohio 03);

FIG. 4B illustrates pALVAC C5 H6p-synthetic EIV H3 HA, pJY1571.1;

FIG. 5A illustrates a predicted amino acid sequence of product(s): EIV H3 HA;

FIG. 5B illustrates a nucleotide sequence of arms and insert with translation

FIG. 6 illustrates a vCP2242 Western blot analysis. A 1/1,000 dilution of pooled anti-EIV antibody was used for the analysis. Lane 1: 5 μl Fermentas Prestain protein marker, lane 2: 15 μl ALVAC cell pellet, lane 3: 15 μl vCP2242. cell pellet, lane 4: 15 μl vCP2242. cell pellet, lane 5: 15 μl vCP1533 cell pellet, lane 6: space, lane 7: 40 μl ALVAC supernatant, lane 8: 40 μl vCP2242. supernatant, lane 9: 40 μl vCP2242. supernatant and lane 10: 40 μl vCP1533 supernatant.

FIG. 7 illustrates the HI antibody responses to canine influenza virus (CIV) in dogs vaccinated with canarypox-vectored equine influenza virus (EIV) vaccines expressing either the hemagglutinin gene of A/equine/Kentucky/94 (vCP1529) or the hemagglutin gene of A2/equine/Ohio/03 (vCP2242). vCP2242 was administered by the subcutaneous (SC in FIG. 7) or the transdermal route (TD in FIG. 7) whereas vCP1529 was administered subcutaneously (SC in FIG. 7).

FIG. 8 illustrates the mean viral titers found in nasal swabs in control animals and vaccinated animals following intranasal challenge with influenza A/canine/NY/110659. Data=log10 virus isolation titers on MDCK cells.

FIG. 9 illustrates the mean body temperatures of control dogs and of dogs vaccinated subcutaneously twice at a 21-day interval with the hemagglutin gene of A2/equine/Ohio/03 (vCP2242) followed by challenge at day 43 with CIV-NY05 isolate (influenza A/canine/NY/110659).

FIG. 10 illustrates the mean body temperatures of control dogs (CON) and of dogs vaccinated subcutaneously twice at a 21-day interval with the hemagglutin gene of A2/equine/Ohio/03 (vCP2242) (VAC) followed by challenge at day 43 with CIV-NY05 isolate on days 42, 44, and 45. This figure is a box plot that has the mean (symbolized as a +) and the median (horizontal line), the 25^{th} and 75^{th} percentile (bottom and top of the box), minimum and maximum observations (whiskers), and outliers (small squares).

FIG. 11 illustrates the HI antibody responses to CIV HA antigen of A2/equine/Ohio/03 (vCP2242) followed by challenge at day 43 with CIV-NY05 isolate at three weeks post second vaccination and two weeks post challenge (Vac57), and in control dogs at two weeks post challenge (Cont57).

FIG. 12 illustrates the protective immune response of vCP2242 as characterized by the decreased time of virus shedding in the nasal cavity of dogs vaccinated subcutaneously twice at a 21-day interval with the hemagglutin gene of A2/equine/Ohio/03 (vCP2242) followed by challenge at day 43 with CIV-NY05 isolate as compared to control dogs.

FIG. 13 illustrates the protective immune response of vCP2242 as characterized by the decreased duration of virus shedding in the nasal cavity of dogs vaccinated subcutaneously twice at a 21-day interval with the hemagglutin gene of A2/equine/Ohio/03 (vCP2242) followed by challenge at day 43 with CIV-NY05 isolate as compared to control dogs.

### DETAILED DESCRIPTION

The present invention is based, in part, on Applicants' studies demonstrating a recombinant canarypox expressing equine influenza HA is immunogenic in dogs.

The present invention encompasses any influenza antigen, epitope or immunogen that elicits an immunogenic response in an animal, advantageously a vertebrate, more advantageously a dog. The influenza antigen, epitope or immunogen may be any influenza antigen, epitope or immunogen, such as, but not limited to, a protein, peptide or fragment thereof, that elicits, induces or stimulates a response in an animal, advantageously a vertebrate, more advantageously a dog.

In an advantageous embodiment, the canine influenza antigen, epitope or immunogen is derived from a canine infected with influenza. For example, but not by limitation, influenza virus may be isolated from the broncho alveolar lavage and/or lung tissues of an affected dog. Isolation and characterization of the nucleotide sequence of the influenza infecting the dog may be done by routine experimentation by a person of ordinary skill in the art.

In another advantageous embodiment, the canine influenza, antigen, epitope or immunogen may be derived from an equine infected with influenza or an equine influenza strain. Advantageously, the equine influenza strain is an Ohio equine influenza, Kentucky equine influenza strain or a Newmarket equine influenza strain. The canine influenza antigen, epitope or immunogen may be determined by one of ordinary skill of the art from the nucleotide sequences of Examples 4 and 5. Advantageously, the canine influenza antigen, epitope or immunogen is a hemagglutinin (HA) (e.g., HA precursor, H1, H2, protein, matrix protein (e.g., matrix protein M1 or M2), neuraminidase, nonstructural (NS) protein (e.g., NS1 or NS2), nucleoprotein (NP) and polymerase (e.g., PA polymerase, PB1 polymerase 1 or PB2 polymerase 2).

Examples of Kentucky equine influenza strains that may be used in methods of the present invention include, but are not limited to, equine influenza strains A/eq/Kentucky/98 (see, e.g., Crouch et al., Vaccine. 2004 Dec. 2; 23(3):418-25), A/Equi 2 (Kentucky 81) (see, e.g., Short et al., J Vet Pharmacol Ther. 1986 December; 9(4):426-32, Horner & Ledgard, N Z Vet J. 1988 December; 36(4):205-6), A/equine/Kentucky/1/81 (Eq/Ky) (see, e.g., Breathnach et al., Vet Immunol Immunopathol. 2004 April; 98(3-4):127-36), A/Equine/Kentucky/1/81 (H3N8) (see, e.g., Olsen et al., Vaccine. 1997 July; 15(10):1149-56, Morley et al. Vet Microbiol. 1995 June; 45(1):81-92, Ozaki et al., Vet Microbiol. 2001 Sep. 20; 82(2):111-9, Sugiura et al., J Virol Methods. 2001 October; 98(1):1-8, see, e.g., Sugiura et al., J Virol Methods. 2001 October; 98(1):1-8, Goto et al., J Vet Med. Sci. 1993 February; 55(1):33-7, Goto et al., J Vet Med. Sci. 1993 February; 55(1):33-7), A/Equine/Kentucky/1/91 (H3N8) (see, e.g., Youngner et al., Am J Vet Res. 2001 August; 62(8):1290-4), A/Equine/Kentucky/1277/90 (Eq/Kentucky) (see, e.g., Webster & Thomas, Vaccine. 1993; 11(10):987-93), A/Equine/Kentucky/2/91 (H3N8) (see, e.g., Donofrio et al., J Vet Diagn Invest. 1994 January; 6(1):39-43), A/Equine/Kentucky/79 (H3N8) (see, e.g., Donofrio et al., J Vet Diagn Invest. 1994 January; 6(1):39-43), A/equine/Kentucky/81 (see, e.g., Sugiura et al., J Virol Methods. 2001 October; 98(1):1-8), A/equine/Kentucky/91 (H3N8) (see, e.g., Gross et al., Equine Vet J. 1998 November; 30(6):489-97), A/equine-2/Kentucky/95 (H3N8) (see, e.g., Heldens et al., Vet J. 2004 March; 167(2):150-7) and A/equine-2/Kentucky/98 (see, e.g., Chambers et al., Equine Vet J. 2001 November; 33(7):630-6), the disclosures of which are incorporated by reference in their entireties.

Examples of Newmarket equine influenza strains that may be used in methods of the present invention include, but are not limited to, equine influenza strains A/eq/Newmarket/1/77 (see, e.g., Lindstrom et al., Arch Virol. 1998; 143(8):1585-98), A/eq/Newmarket/5/03 (see, e.g., Edlund Toulemonde et al., Vet Rec. 2005 Mar. 19; 156(12):367-71), A/Equi 2 (H3N8), Newmarket 1/93 (see, e.g., Mohler et al., Biotechnol Bioeng. 2005 Apr. 5; 90(1):46-58, Nayak et al., J Chromatogr B Analyt Technol Biomed Life Sci. 2005 Jul 8), A/equi-2/Newmarket-1/93 (see, e.g., Heldens et al., J Immunol Methods. 2002 Jun. 1; 264(1-2):11-7), A/equine/Newmarket/2/93 (see, e.g., Wattrang et al., Viral Immunol. 2003; 16(1):57-67), A/equine/Newmarket/79 (H3N8) (see, e.g., Duhaut & Dimmock, Virology. 2000 Sep. 30; 275(2):278-85, Noble & Dimmock, J Gen Virol. 1994 December; 75 (Pt 12):3485-91, Duhaut & Dimmock, Virology. 1998 Sep. 1; 248(2):241-53, Hannant & Mumford, Vet Immunol Immunopathol. 1989 July; 21(3-4):327-37, Hannant et al., Vet Microbiol. 1989 April; 19(4):293-303, Hannant et al., Vet Rec. 1988 Feb. 6; 122(6):125-8, Richards et al., Vet Immunol Immunopathol. 1992 June; 33(1-2):129-43, Heldens et al., Vet J. 2004 March; 167(2):150-7), A/equine/Newmarket/1/77 (H7N7) (see, e.g., Goto et al., J Vet Med. Sci. 1993 February; 55(1):33-7, Sugiura et al., J Virol Methods. 2001 October; 98(1):1-8, Sugiura et al., J Virol Methods. 2001 October; 98(1):1-8) and A/equine-2/Newmarket-2/93 (see, e.g., Heldens et al., Vet J. 2004 March; 167(2):150-7), the disclosures of which are incorporated by reference in their entireties.

The present invention also encompasses other equine influenza viruses, such as, but not limited to, equine influenza virus A/eq/Miami/63 (H3N8) (see, e.g., van Maanen et al., Vet Microbiol. 2003 Jun. 10; 93(4):291-306), A/equi 1 (Prague strain) (see, e.g., Horner & Ledgard, N Z Vet J. 1988 December; 36(4):205-6, Short et al., J Vet Pharmacol Ther. 1986 December; 9(4):426-32), A/Equi 2 (Miami) (see, e.g., Short et al., J Vet Pharmacol Ther. 1986 December; 9(4):426-32), A/equi-1/Prague/56 (Pr/56) (see, e.g., Heldens et al., J Immunol Methods. 2002 Jun. 1; 264(1-2):11-7), A/equi-2/Suffolk/89 (Suf/89) (see, e.g., Heldens et al., J Immunol Methods. 2002 Jun. 1; 264(1-2):11-7), A/Equine 2/Sussex/89 (H3N8) (see, e.g., Mumford et al., Vet Rec. 1994 Feb. 12; 134(7):158-62), A/equine/Sussex/89 (see, e.g., Wattrang et al., Viral Immunol. 2003; 16(1):57-67), A/equine-2/Saskatoon/90 (see, e.g., Chambers et al., Equine Vet J. 2001 November; 33(7):630-6), A/Equine/Prague/1/56 (H7N7) (see, e.g., Donofrio et al., J Vet Diagn Invest. 1994 January; 6(1):39-43, Morley et al. Vet Microbiol. 1995 June; 45(1):81-92), A/equine/Miami/1/63 (H3N8) (see, e.g., Morley et al. Vet Microbiol. 1995 June; 45(1):81-92, Ozaki et al., Vet Microbiol. 2001 Sep. 20; 82(2):111-9, Thomson et al., Vet Rec. 1977 May 28; 100(22):465-8, Mumford et al., Epidemiol Infect. 1988 June; 100(3):501-10, Donofrio et al., J Vet Diagn Invest. 1994 January; 6(1):39-43, Mumford et al., J Hyg (Lond). 1983 June; 90(3):385-95), A/Aichi/2/68 (H3N2) (see, e.g., Ozaki et al., Vet Microbiol. 2001 Sep. 20; 82(2):111-9), A/equine/Tokyo/2/71 (H3N8) (see, e.g., Goto et al., J Vet Med. Sci. 1993 February; 55(1):33-7), A/eq/LaPlata/1/88 (see, e.g., Lindstrom et al., Arch Virol. 1998; 143(8):1585-98), A/Equine/Jilin/1/89 (Eq/Jilin) (see, e.g., Webster & Thomas, Vaccine. 1993; 11(10):987-93), A/Equine/Alaska/1/91 (H3N8) (see, e.g., Webster & Thomas, Vaccine. 1993; 11(10):987-93), A/equine/Saskatoon/1/91 (H3N8) (see, e.g., Morley et al. Vet Microbiol. 1995 June; 45(1):81-92), A/equine/Rome/5/91 (H3N8) (see, e.g., Sugiura et al., J Virol Methods. 2001 October; 98(1):1-8), A/equine/La Plata/1/93 (H3N8) (see, e.g., Ozaki et al., Vet Microbiol. 2001 Sep. 20; 82(2):111-9), A/equine/La Plata/1/93 (LP/93) (see, e.g., Sugiura et al., J Virol Methods. 2001 October; 98(1):1-8), A/eq/Holland/1/95 (H3N8) (see, e.g., van Maanen et al., Vet Microbiol. 2003 Jun. 10; 93(4):291-306) and A/eq/Holland/2/95 (H3N8) (see, e.g., van Maanen et al., Vet Microbiol. 2003 Jun. 10; 93(4):291-306), the disclosures of which are incorporated by reference in their entireties.

In another advantageous embodiment, the canine influenza, antigen, epitope or immunogen may be derived from an equine infected with influenza or an equine influenza strain derived from a recent isolate.

The influenza antigen, epitope or immunogen may also be isolated from any influenza strain such as, but not limited to, avian H5N1 influenza virus, A/Hong Kong/156/97 (A/HK/156/97) (see, e.g., Leneva et al., Antimicrob Agents Chemother. 2001 April; 45(4):1216-24), avian H7N1 influenza strain (see, e.g., Foni et al., New Microbiol. 2005 January; 28(1):31-5), avian H9N2 influenza virus (see, e.g., Leneva et al., Antimicrob Agents Chemother. 2001 April; 45(4): 1216-24), avian influenza virus A/Chicken/HK/G9/97 (H9N2) (see, e.g., Leneva et al., Antimicrob Agents Chemother. 2001 April; 45(4): 1216-24), avian influenza virus A/Quail/HK/G1/97 (H9N2) (see, e.g., Leneva et al., Antimicrob Agents Chemother. 2001 April; 45(4):1216-24), avian influenza virus A/Teal/HK/W312/97 (H6N1) (see, e.g., Leneva et al., Antimicrob Agents Chemother. 2001 April; 45(4):1216-24), avian pandemic influenza A viruses of avian origin (see, e.g., Audsley & Tannock, Expert Opin Biol Ther. 2004 May; 4(5):709-17), cold-adapted (ca) and temperature sensitive (ts) master donor strain, A/Leningrad/134/17/57 (H2N2) (see, e.g., Youil et al., Virus Res. 2004 Jun. 15; 102(2):165-76), equine influenza virus (A/Equi 2 (H3N8), Newmarket 1/93) (see, e.g., Mohler et al., Biotechnol Bioeng. 2005 Apr. 5; 90(1):46-58; Nayak et al., J Chromatogr B Analyt Technol Biomed Life Sci. 2005 Jul. 8), equine-2 influenza virus (EIV; subtype H3N8) (see, e.g., Virology. 2001 Aug. 15; 287(1):202-13), equine-2 influenza virus, A/Equine/Kentucky/1/91 (H3N8) (see, e.g., Youngner et al., Am J Vet Res. 2001 August; 62(8):1290-4), human influenza virus A(H3N2) isolates (see, e.g., Abed et al., J Infect Dis. 2002 Oct. 15; 186(8):1074-80), human influenza virus A/Memphis/1/71 (H3N2) (see, e.g., Suzuki et al., Biochem J. 1996 Sep. 1; 318 (Pt 2):389-93), human influenza virus A/Nanchang/933/95 (H3N2) virus (see, e.g., Scholtissek et al., J. Virol. 2002 February; 76(4):1781-6), human influenza virus A/PR/8/34 (H1N1) virus (see, e.g., Scholtissek et al., J. Virol. 2002 February; 76(4):1781-6), human influenza virus A/Singapore/57 (H2N2) virus (see, e.g., Scholtissek et al., J. Virol. 2002 February; 76(4):1781-6), influenza virus A (see, e.g., Chare et al., J Gen Virol. 2003 October; 84(Pt 10):2691-703), influenza virus A PR/8/34 (PR8) virus (H1N1 subtype) (see, e.g., Mantani et al., Planta Med. 2001 April; 67(3):240-3), influenza virus A/Aichi/2/68(H3N2) (see, e.g., Miyamoto et al., Antiviral Res. 1998 August; 39(2):89-100), influenza virus A/Ann Arbor/6/60 cold-adapted virus (see, e.g., Treanor et al., J. Virol. 1994 December; 68(12):7684-8), influenza virus A/Beijing 32/92 (H3N2) (see, e.g., Zakay-Rones et al., J Altern Complement Med. 1995 Winter; 1(4):361-9), influenza virus A/Charlottesville/31/95 (H1N1) (see, e.g., Gubareva et al., J Gen Virol. 2002 November; 83(Pt 11):2683-92), influenza virus A/Kawasaki/86 (H1N1) virus (see, e.g., Staschke et al., Virology. 1998 Sep. 1; 248(2):264-74), influenza virus A/Korea/82 (H3N2) (see, e.g., Treanor et al., J. Virol. 1994 December; 68(12):7684-8), influenza virus A/Leningrad/134/57 (see, e.g., Egorov et al., J. Virol. 1998 August; 72(8):6437-41), influenza virus A/NWS/33 (H1N1) (see, e.g., Sidwell et al., Antiviral Res. 1998 February; 37(2):107-20), influenza virus A/PR/8/34(H1N1) (see, e.g., Miyamoto et al., Antiviral Res. 1998 August; 39(2):89-100), influenza virus A/PR8/34 (see, e.g., Nunes-Correia et al., Biochemistry. 1999 Jan. 19; 38(3):1095-101, Tree et al., Vaccine. 2001 May 14; 19(25-26):3444-50), influenza virus A/Puerto Rico (PR)/8/34 (see, e.g., Egorov et al., J. Virol. 1998 August; 72(8):6437-41), influenza virus A/Puerto Rico/8-Mount Sinai (see, e.g., Mazanec et al., J. Virol. 1995 February; 69(2):1339-43), influenza virus A/Shangdong 9/93 (H3N2) (see, e.g., Zakay-Rones et al., J Altem Complement Med. 1995 Winter; 1(4):361-9, Sidwell et al., Antiviral Res. 1998 February; 37(2):107-20), influenza virus A/Shingapol/1/57(H2N2) (see, e.g., Miyamoto et al., Antiviral Res. 1998 August; 39(2):89-100), influenza virus A/Singapore 6/86 (H1N1) (see, e.g., Zakay-Rones et al., J Altem Complement Med. 1995 Winter; 1(4):361-9), influenza virus A/Singapore/1/57 (H2N2) (see, e.g., Bantia et al., Antimicrob Agents Chemother. 1998 April; 42(4):801-7), influenza virus A/Texas 36/91 (H1N1) (see, e.g., Zakay-Rones et al., J Altern Complement Med. 1995 Winter; 1(4):361-9), influenza virus A/Texas/36/91 (H1N1) virus (see, e.g., Gubareva et al., J Infect Dis. 2001 Feb. 15; 183(4):523-31, Halperin et al., Vaccine. 1998 August; 16(13):1331-5), influenza virus A/Texas/36/91(H1N1) (see, e.g., Hayden et al., Antiviral Res. 1994 October; 25(2):123-31), influenza virus A/Udorn/72 virus infection (see, e.g., Shimizu et al., Virology. 1999 Feb. 15; 254(2):213-9), influenza virus A/Victoria/3/75 (H3N2) (see, e.g., Sidwell et al., Antiviral Res. 1998 February; 37(2):107-20), influenza virus A/Virginia/88(H3N2) (see, e.g., Hayden et al., Antiviral Res. 1994 October; 25(2):123-31), influenza virus A/WSN/33 (H1N1) (see, e.g., Lu et al., Arch Virol. 2002; 147(2):273-84), influenza virus A/WSN/33 (see, e.g., Gujuluva et al., Virology. 1994 Nov. 1; 204(2):491-505), influenza virus B (see, e.g., Chare et al., J Gen Virol. 2003 October; 84(Pt 10):2691-703), influenza virus B/Ann Arbor 1/86 (see, e.g., Zakay-Rones et al., J Altern Complement Med. 1995 Winter; 1(4):361-9), influenza virus B/Harbin/7/94 (see, e.g., Halperin et al., Vaccine. 1998 August; 16(13):1331-5, influenza virus B/Hong Kong/5/72 (see, e.g., Sidwell et al., Antiviral Res. 1998 February; 37(2):107-20), influenza virus B/Lee/40 (see, e.g., Miyamoto et al., Antiviral Res. 1998 August; 39(2):89-100), influenza virus B/Victoria group (see, e.g., Nakagawa et al., J Virol Methods. 1999 April; 79(1): 113-20), influenza virus B/Yamagata 16/88 (see, e.g., Zakay-Rones et al., J Altern Complement Med. 1995 Winter; 1(4):361-9), influenza virus B/Yamagata group (see, e.g., Nakagawa et al., J Virol Methods. 1999 April; 79(1): 113-20), influenza virus B/Yamanashi/166/98 (see, e.g., Hoffmann et al., Proc Natl Acad Sci USA. 2002 Aug. 20; 99(17):11411-6), influenza virus C (see, e.g., Chare et al., J Gen Virol. 2003 October; 84(Pt 10):2691-703), influenza virus strain A/Equi/2/Kildare/89 (see, e.g., Quinlivan et al., J Clin Microbiol. 2004 February; 42(2):759-63), influenza virus type B/Panama 45/90 (see, e.g., Zakay-Rones et al., J Altem Complement Med. 1995 Winter; 1(4):361-9), live, cold-adapted, temperature-sensitive (ca/ts) Russian influenza A vaccines (see, e.g., Palker et al., Virus Res. 2004 October; 105(2):183-94), Madin Darby Canine Kidney (MDCK)-derived cell line (see, e.g., Halperin et al., Vaccine. 2002 Jan. 15; 20(7-8):1240-7), mouse-adapted influenza virus A/Guizhou/54/89 (H3N2 subtype) (see, e.g., Nagai et al., Biol Pharm Bull. 1995 February; 18(2):295-9), mouse-adapted influenza virus A/PR/8/34 (A/PR8) (see, e.g., Nagai et al., Antiviral Res. 1995 January; 26(1): 11-25), mouse-adapted influenza virus B/Ibaraki/2/85 (see, e.g., Nagai et al., Biol Pharm Bull. 1995 February; 18(2):295-9), Russian live attenuated influenza vaccine donor strains A/Leningrad/134/17/57, A/Leningrad/134/47/57 and B/USSR/60/69 (see, e.g., Audsley & Tannock, J Virol Methods. 2005 February; 123(2):187-93), swine H1 and H3 influenza viruses (see, e.g., Gambaryan et al., Virus Res. 2005 Jul. 1), swine influenza A viruses (see, e.g., Landolt et al., Am J Vet Res. 2005 January; 66(1): 119-24), swine influenza virus (SIV) (see, e.g., Clavijo et al., Can J Vet Res. 2002 April; 66(2): 117-21), swine influenza virus A/Sw/Ger 2/81 (see, e.g., Zakay-Rones et al., J Altern Complement Med. 1995 Winter; 1(4):361-9), swine influenza virus A/Sw/Ger 8533/91 (see, e.g., Zakay-Rones et al., J Altern Complement Med. 1995 Winter; 1(4):361-9), turkey influenza virus A/Tur/Ger 3/91 (see, e.g., Zakay-Rones et al., J Altern Complement Med. 1995 Winter; 1(4):361-9) and turkey influenza virus A/turkey/Minnesota/833/80 (H4N2) (see, e.g., Gubareva et al., J. Virol. 1997 May; 71(5):3385-90), the disclosures of which are incorporated by reference.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may comprise a toxin or antitoxin.

The term “immunogenic protein or peptide” as used herein also refers includes peptides and polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

The term “immunogenic protein or peptide” further contemplates deletions, additions and substitutions to the sequence, so long as the polypeptide functions to produce an immunological response as defined herein. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The terms “immunogenic” protein or polypeptide as used herein also refers to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Methods especially applicable to the proteins of T. parva are fully described in the PCT Application Serial No. PCT/US2004/022605 incorporated herein by reference in its entirety.

Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998. Immunogenic fragments, for purposes of the present invention, will usually include at least about 3 amino acids, preferably at least about 5 amino acids, more preferably at least about 10-15 amino acids, and most preferably 25 or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it comprises or consists essentially of or consists of nucleotides to encode an epitope or antigenic determinant of an influenza protein or polyprotein. A polynucleotide encoding a fragment of the total protein or polyprotein, more advantageously, comprises or consists essentially of or consists of a minimum of 21 nucleotides, advantageously at least 42 nucleotides, and preferably at least 57, 87 or 150 consecutive or contiguous nucleotides of the sequence encoding the total protein or polyprotein. Epitope determination procedures, such as, generating overlapping peptide libraries (Hemmer B. et al., Immunology Today, 1998, 19 (4), 163-168), Pepscan (Geysen et al., (1984) Proc. Nat. Acad. Sci. USA, 81, 3998-4002; Geysen et al., (1985) Proc. Nat. Acad. Sci. USA, 82, 178-182; Van der Zee R. et al., (1989) Eur. J. Immunol., 19, 43-47; Geysen H. M., (1990) Southeast Asian J. Trop. Med. Public Health, 21, 523-533; Multipin.®. Peptide Synthesis Kits de Chiron) and algorithms (De Groot A. et al., (1999) Nature Biotechnology, 17, 533-561), and in PCT Application Serial No. PCT/US2004/022605 all of which are incorporated herein by reference in their entireties, can be used in the practice of the invention, without undue experimentation. Other documents cited and incorporated herein may also be consulted for methods for determining epitopes of an immunogen or antigen and thus nucleic acid molecules that encode such epitopes.

A “polynucleotide” is a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The invention further comprises a complementary strand to a polynucleotide encoding an influenza antigen, epitope or immunogen. The complementary strand can be polymeric and of any length, and can contain deoxyribonucleotides, ribonucleotides, and analogs in any combination.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

Hybridization reactions can be performed under conditions of different “stringency.” Conditions that increase stringency of a hybridization reaction are well known. See for example, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The invention further encompasses polynucleotides encoding functionally equivalent variants and derivatives of the influenza polypeptides and functionally equivalent fragments thereof which may enhance, decrease or not significantly affect properties of the polypeptides encoded thereby. These functionally equivalent variants, derivatives, and fragments display the ability to retain influenza activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the influenza polynucleotide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as

(N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The invention further encompasses the influenza polynucleotides contained in a vector molecule or an expression vector and operably linked to a promoter element and optionally to an enhancer.

The vector is advantageously a poxvirus, particularly a vaccinia virus or an avipox virus, such as fowlpox virus and canarypox virus. Advantageously, the virus is a canarypox virus. Advantageous canarypox strains may be an attenuated strain. The vector can express at least one epitope from Kentucky strains, Newmarket strains and/or Ohio strains. Advantageous canarypox constructs include, but are not limited to, vCP 1529, vCP 1533 and vCP 2242. Recombinant avipox viruses (see, e.g., U.S. Pat. Nos. 5,505,941 and 5,756,103), such as an attenuated recombinant canarypox virus, for instance ALVAC, or an attenuated fowlpox virus, for instance TROVAC, are especially advantageous. In one advantageous embodiment, the recombinant ALVAC vaccine described by Edlund Toulemonde et al., Vet Rec. 2005 Mar. 19; 156(12):367-71 may be used as a canine influenza vaccine. Other viruses that may be used in methods of the invention include, but are not limited to, vaccinia viruses, such as an attenuated vaccinia virus, for instance NYVAC, adenoviruses, such as canine adenoviruses (CAV), and herpesviruses, such as canine herpesvirus (CHV) or a feline herpesvirus (FHV).

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector needs not be capable of replication in the ultimate target cell or subject. The term includes cloning vectors also included are viral vectors.

The term “recombinant” means a polynucleotide semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

Elements for the expression of an influenza antigen, epitope or immunogen are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. an influenza peptide, advantageously, in the vector, an ATG is placed at 5′ of the reading frame and a stop codon is placed at 3′. Other elements for controlling expression may be present, such as enhancer sequences, stabilizing sequences, such as intron and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6; 312,683; U.S. patent application Serial No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., human adenovirus, canine adenovirus), herpesvirus (e.g. canine herpesvirus), baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-influenza peptides or fragments thereof to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of influenza antigens, epitopes or immunogens. Advantageously, the vector contains and expresses a polynucleotide that includes, consists essentially of, or consists of a polynucleotide coding for (and advantageously expressing) an influenza antigen, epitope or immunogen, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of an influenza antigen, epitope or immunogen (e.g., hemagglutinin, neuraminidase, nucleoprotein) or a fragment thereof.

According to another embodiment, the vector or vectors in the preparation comprise, or consist essentially of, or consist of polynucleotide(s) encoding one or more proteins or fragment(s) thereof of an influenza antigen, epitope or immunogen, the vector or vectors expressing the polynucleotide(s). The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different canine influenza isolates encoding the same proteins and/or for different proteins, but advantageously the same proteins. Preparations containing one or more vectors containing, consisting essentially of or consisting of polynucleotides encoding, and advantageously expressing, advantageously in vivo, an influenza antigen, fusion protein or an epitope thereof. The invention is also directed at mixtures of vectors that contain, consist essentially of, or consist of coding for, and express, different influenza antigens, epitopes or immunogens, e.g., an influenza antigen, epitope or immunogen from different species such as, but not limited to, humans, pigs, in addition to avian species including chicken, ducks and geese.

According to one embodiment of the invention, the expression vector is a viral vector, in particular an in vivo expression vector. In an advantageous embodiment, the expression vector is an adenovirus vector. Advantageously, the adenovirus is a human Ad5 vector, an E1-deleted and/or an E3-deleted adenovirus.

In one particular embodiment the viral vector is a poxvirus, e.g. a vaccinia virus or an attenuated vaccinia virus, (for instance, MVA, a modified Ankara strain obtained after more than 570 passages of the Ankara vaccine strain on chicken embryo fibroblasts; see Stickl & Hochstein-Mintzel, Munch. Med. Wschr., 1971, 113, 1149-1153; Sutter et al., Proc. Natl. Acad. Sci. U.S.A., 1992, 89, 10847-10851; available as ATCC VR-1508; or NYVAC, see U.S. Pat. No. 5,494,807, for instance, Examples 1 to 6 and et seq of U.S. Pat. No. 5,494,807 which discuss the construction of NYVAC, as well as variations of NYVAC with additional ORFs deleted from the Copenhagen strain vaccinia virus genome, as well as the insertion of heterologous coding nucleic acid molecules into sites of this recombinant, and also, the use of matched promoters; see also WO96/40241), an avipox virus or an attenuated avipox virus (e.g., canarypox, fowlpox, dovepox, pigeonpox, quailpox, ALVAC or TROVAC; see, e.g., U.S. Pat. Nos. 5,505,941, 5,494,807), swinepox, raccoonpox, camelpox, or myxomatosis virus.

According to another embodiment of the invention, the poxvirus vector is a canarypox virus or a fowlpox virus vector, advantageously an attenuated canarypox virus or fowlpox virus. In this regard, is made to the canarypox available from the ATCC under access number VR-111. Attenuated canarypox viruses are described in U.S. Pat. No. 5,756,103 (ALVAC) and WO01/05934. Numerous fowlpox virus vaccination strains are also available, e.g. the DIFTOSEC CT strain marketed by MERIAL and the NOBILIS VARIOLE vaccine marketed by INTERVET; and, reference is also made to U.S. Pat. No. 5,766,599 which pertains to the attenuated fowlpox strain TROVAC.

For information on the method to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No. 5,766,599 inter alia; as to canarypox mentionis made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus area advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Advantageously, the polynucleotide to be expressed is inserted under the control of a specific poxvirus promoter, e.g., the vaccinia promoter 7.5 kDa (Cochran et al., J. Virology, 1985, 54, 30-35), the vaccinia promoter I3L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

In a particular embodiment the viral vector is an adenovirus, such as a human adenovirus (HAV) or a canine adenovirus (CAV).

In one embodiment the viral vector is a human adenovirus, in particular a serotype 5 adenovirus, rendered incompetent for replication by a deletion in the E1 region of the viral genome, in particular from about nucleotide 459 to about nucleotide 3510 by reference to the sequence of the hAd5 disclosed in Genbank under the accession number M73260 and in the referenced publication J. Chroboczek et al Virol. 1992, 186, 280-285. The deleted adenovirus is propagated in E1-expressing 293 (F. Graham et al J. Gen. Virol. 1977, 36, 59-72) or PER cells, in particular PER.C6 (F. Falloux et al Human Gene Therapy 1998, 9, 1909-1917). The human adenovirus can be deleted in the E3 region, in particular from about nucleotide 28592 to about nucleotide 30470. The deletion in the E1 region can be done in combination with a deletion in the E3 region (see, e.g. J. Shriver et al. Nature, 2002, 415, 331-335, F. Graham et al Methods in Molecular Biology Vol 0.7: Gene Transfer and Expression Protocols Edited by E. Murray, The Human Press Inc, 1991, p 109-128; Y. Ilan et al Proc. Natl. Acad. Sci. 1997, 94, 2587-2592; U.S. Pat. No. 6,133,028; U.S. Pat. No. 6,692,956; S. Tripathy et al Proc. Natl. Acad. Sci. 1994, 91, 11557-11561; B. Tapnell Adv. Drug Deliv. Rev. 1993, 12, 185-199; X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci (region) eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter), in particular the enhancer/promoter region from about nucleotide −734 to about nucleotide +7 in M. Boshart et al Cell 1985, 41, 521-530 or the enhancer/promoter region from the pCI vector from Promega Corp. The CMV-IE promoter is advantageously of murine or human origin. The promoter of the elongation factor 1α can also be used. A muscle specific promoter can also be used (X. Li et al Nat. Biotechnol. 1999, 17, 241-245). Strong promoters are also discussed herein in relation to plasmid vectors. In one embodiment, a splicing sequence can be located downstream of the enhancer/promoter region. For example, the intron 1 isolated from the CMV-IE gene (R. Stenberg et al J. Virol. 1984, 49, 190), the intron isolated from the rabbit or human β-globin gene, in particular the intron 2 from the b-globin gene, the intron isolated from the immunoglobulin gene, a splicing sequence from the SV40 early gene or the chimeric intron sequence isolated from the pC1 vector from Promege Corp. comprising the human β-globin gene donor sequence fused to the mouse immunoglobulin acceptor sequence (from about nucleotide 890 to about nucleotide 1022 in Genbank under the accession number CVU47120). A poly(A) sequence and terminator sequence can be inserted downstream the polynucleotide to be expressed, e.g. a bovine growth hormone gene, in particular from about nucleotide 2339 to about nucleotide 2550 in Genbank under the accession number BOVGHRH, a rabbit β-globin gene or a SV40 late gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and/or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

In another particular embodiment the viral vector is a herpesvirus such as a canine herpesvirus (CHV) or a feline herpesvirus (FHV). For CHV, the insertion sites may be in particular in the thymidine kinase gene, in the ORF3, or in the UL43 ORF (see U.S. Pat. No. 6,159,477). In one embodiment the polynucleotide to be expressed is inserted under the control of a promoter functional in eukaryotic cells, advantageously a CMV-IE promoter (murine or human). A poly(A) sequence and terminator sequence can be inserted downstream the polynucleotide to be expressed, e.g. bovine growth hormone or a rabbit β-globin gene polyadenylation signal.

According to a yet further embodiment of the invention, the expression vector is a plasmid vector or a DNA plasmid vector, in particular an in vivo expression vector. In a specific, non-limiting example, the pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217, see, e.g., U.S. Pat. Nos. 5,846,946 and 6,451,769) can be utilized as a vector for the insertion of a polynucleotide sequence. The pVR1020 plasmid is derived from pVR1012 and contains the human tPA signal sequence. In one embodiment the human tPA signal comprises from amino acid M(1) to amino acid S(23) in Genbank under the accession number HUMTPA14. In another specific, non-limiting example, the plasmid utilized as a vector for the insertion of a polynucleotide sequence can contain the signal peptide sequence of equine IGF1 from amino acid M(24) to amino acid A(48) in Genbank under the accession number U28070. Additional information on DNA plasmids which may be consulted or employed in the practice are found, for example, in U.S. Pat. Nos. 6,852,705; 6,818,628; 6,586,412; 6,576,243; 6,558,674; 6,464,984; 6,451,770; 6,376,473 and 6,221,362.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Each plasmid comprises or contains or consists essentially of, in addition to the polynucleotide encoding an influenza antigen, epitope or immunogen, optionally fused with a heterologous peptide sequence, variant, analog or fragment, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently includes derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Preferably, the plasmids comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit β-globin gene (van Ooyen et al., Science, 1979, 206, 337-344).

As to the polyadenylation signal (polyA) for the plasmids and viral vectors other than poxviruses, use can more be made of the poly(A) signal of the bovine growth hormone (bGH) gene (see U.S. Pat. No. 5,122,458), or the poly(A) signal of the rabbit β-globin gene or the poly(A) signal of the SV40 virus.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof. Advantageous host cells include, but are not limited to, baby hamster kidney (BHK) cells, colon carcinoma (Caco-2) cells, COS7 cells, MCF-7 cells, MCF-10A cells, Madin-Darby canine kidney (MDCK) lines, mink lung (Mv1Lu) cells, MRC-5 cells, U937 cells and VERO cells. Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In an advantageous embodiment, the invention provides for the administration of a therapeutically effective amount of a formulation for the delivery and expression of an influenza antigen, epitope or immunogen in a target cell. Determination of the therapeutically effective amount is routine experimentation for one of ordinary skill in the art. In one embodiment, the formulation comprises an expression vector comprising a polynucleotide that expresses an influenza antigen, epitope or immunogen and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The cationic lipids containing a quaternary ammonium salt which are advantageously but not exclusively suitable for plasmids, are advantageously those having the following formula:

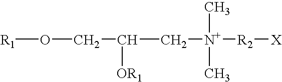

in which R₁ is a saturated or unsaturated straight-chain aliphatic radical having 12 to 18 carbon atoms, R₂ is another aliphatic radical containing 2 or 3 carbon atoms and X is an amine or hydroxyl group, e.g. the DMRIE. In another embodiment the cationic lipid can be associated with a neutral lipid, e.g. the DOPE.

Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994, Bioconjugate Chemistry, 5, 382-389), to form DMRIE-DOPE.

Advantageously, the plasmid mixture with the adjuvant is formed extemporaneously and advantageously contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is advantageously about 95: about 5 to about 5 about 95, more advantageously about 1: about 1, e.g., 1:1.

The DMRIE or DMRIE-DOPE adjuvant:plasmid weight ratio can be between about 50: about 1 and about 1: about 10, such as about 10: about 1 and about 1 about 5, and advantageously about 1: about 1 and about 1: about 2, e.g., 1:1 and 1:2.

The invention also provides for inactivated canine influenza vaccines. As used herein, the term “inactivated vaccine” means a vaccine composition containing an infectious organism or pathogen that is no longer capable of replication or growth. Inactivation may be accomplished by a variety of methods sufficient to prevent replication or growth of the organism while maintaining its immunogenicity.

The inactivated vaccine may be an inactivated form of an isolate of an influenza virus from an affected dog. The virus may be isolated from the alveoli or lung of an affected dog. In another embodiment, the inactivated vaccine may be an inactivated equine influenza. In an advantageous embodiment, the equine influenza is a Kentucky or Newmarket equine influenza. The inactivated vaccine may be an inactivated version of any one of the influenza strains described above.

An inactivated vaccine may be prepared as well from the harvested culture fluid. The virus may be produced either by inoculation of 10-11-day embryonated eggs (J. Violay et al U.S. Pat. No. 6,048,537) or by inoculation of BHK-21 cell culture (C. Ross et al Archiv. Für die gesamte Virusforschung 1970, 30, 82-88; T. Tolstova et al Acta Virol. 1966, 10, 315-321; Ho. Merten et al Adv. Exp. Med. Biol. 1996, 397, 141-151), of MDCK cell culture (J. Tree et al Vaccine 2001, 19, 3444-3450; Y. Ghendon et al Vaccine 2005, 23, 4678-4684; R. Brands et al Dev. Biol. Stand. 1999, 98, 93-100; R. Youil et al J. Virol. Methods 2004, 120, 23-31), of Vero cell culture (O. Kistner et al Vaccine 1998, 16, 960-968; E. Govorkova et al J. Virol. 1996, 70, 5519-5524). The allantoic fluid or the cell culture supernatant can be clarified by low centrifugation and/or filtration. The virus can be concentrated by ultrafiltration and can be purified by zonal centrifugation on sucrose gradient (J. Violay et al U.S. Pat. No. 6,048,537; O. Kistner et al idem), by gel filtration (D. Nayak et al J. Chromatogr. B Analyt. Technol. Biomed. Life Sci. 2005, 823, 75-81; S. Tomita et al Kitasato Arch. Exp. Med. 1971, 44, 185-196).

Inactivation may be achieved by treating the viruses by any of the methods commonly employed to make inactivated vaccines. These methods include but are not limited to formaldehyde treatment (O. Kistner et al idem; A. Garcia et al Avian Diseases 1998, 42, 248-256), betapropriolactone treatment (B. Bdowsky et al Vaccine 1991, 9, 398-402 and Vaccine 1993, 11, 343-348; N. Keverin et al Arch. Virol. 2000, 145, 1059-1066), ethylene-imine treatment (D. Swayne et al Avian Diseases 2001, 45, 355-365), treatment with organic solvents, treatment with detergents, treatment with Tween-ether or treatment with Triton X-100 (J. Vilay et al idem) for allantoic fluid. For the inactivation the concentration can be about 0.01-0.2% w/v for the formaldehyde; about 0.03-0.2% w/v for the betapropiolactone; about 0.5-20 mM for ethyleneimine. The methods recited herein serve as art-known examples for inactivating virus. Inactivated virus vaccines are usually administered mixed with an adjuvant. The inactivated vaccine can be administered to the animal by any of a plurality of methods which include but are not limited to inoculation intramuscularly, intradermally, or subcutaneously, spraying, ocularly, nasally, orally, or in ovo.

The immunogenic compositions and vaccines according to the invention may comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al., Proc. Natl. Acad. Sci., USA, 1996, 93, 2879-2883; WO98/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, e.g., DDA (5) cytokines, (6) aluminum hydroxide or aluminum phosphate, (7) saponin or (8) other adjuvants discussed in any document cited and incorporated by reference into the instant application, or (9) any combinations or mixtures thereof, or (10) immunostimulating complexes (ISCOMs) that are open cage-like complexes built up by cholesterols, lipids, immunogen, and saponins from the bark of the tree Quillaia saponaria Molina (Sjolander et al., J. Leukocyte Biol., 1998, 64 (6), 713-723).

The oil in water emulsion (3), which is especially appropriate for viral vectors, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil is used in combination with emulsifiers to form an emulsion. The emulsifiers may be nonionic surfactants, such as: esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated, or polyoxypropylene-polyoxyethylene copolymer blocks, such as Pluronic, e.g., L121.

The type (1) immunostimulating complex matrix type (ISCOM™ matrix) adjuvants are advantageous choices according to the invention, especially since it has been shown that this class of adjuvants are capable of up-regulating MHC Class II expression by APCs. An ISCOM™. matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Among the type (1) adjuvant polymers, preference is given to polymers of crosslinked acrylic or methacrylic acid, especially crosslinked by polyalkenyl ethers of sugars or polyalcohols. These compounds are known under the name carbomer (Pharmeuropa, vol. 8, no. 2, June 1996). One skilled in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name Carbopol (BF Goodrich, Ohio, USA) are especially suitable. They are crosslinked by allyl saccharose or by allyl pentaerythritol. Among them, reference is made to Carbopol 974P, 934P and 971P.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., Nature 186: 778-780, Jun. 4, 1960.

With regard to structure, the acrylic or methacrylic acid polymers and EMA are preferably formed by basic units having the following formula:

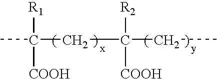

in which:
R₁ and R₂, which can be the same or different, represent H or CH₃
x=0 or 1, preferably x=1
y=1 or 2, with x+y=2.

For EMA, x=0 and y=2 and for carbomers x=y=1.

These polymers are soluble in water or physiological salt solution (20 g/l NaCl) and the pH can be adjusted to 7.3 to 7.4, e.g., by soda (NaOH), to provide the adjuvant solution in which the expression vector(s) can be incorporated. The polymer concentration in the final vaccine composition can range between 0.01 and 1.5% w/v, advantageously 0.05 to 1% w/v and preferably 0.1 to 0.4% w/v.

The cytokine or cytokines (5) can be in protein form in the immunogenic or vaccine composition, or can be co-expressed in the host with the immunogen or immunogens or epitope(s) thereof. Preference is given to the co-expression of the cytokine or cytokines, either by the same vector as that expressing the immunogen or immunogens or epitope(s) thereof, or by a separate vector therefor.

The invention comprehends preparing such combination compositions; for instance by admixing the active components, advantageously together and with an adjuvant, carrier, cytokine, and/or diluent.

Cytokines that may be used in the present invention include, but are not limited to, granulocyte colony stimulating factor (G-CSF), granulocyte/macrophage colony stimulating factor (GM-CSF), interferon α (IFN α), interferon β (IFN β), interferon γ, (IFN γ), interleukin-1α (IL-1α), interleukin-1β (IL-1β), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), tumor necrosis factor α (TNF α), tumor necrosis factor β (TNF β), and transforming growth factor β (TGF β). It is understood that cytokines can be co-administered and/or sequentially administered with the immunogenic or vaccine composition of the present invention. Thus, for instance, the vaccine of the instant invention can also contain an exogenous nucleic acid molecule that expresses in vivo a suitable cytokine, e.g., a cytokine matched to this host to be vaccinated or in which an immunological response is to be elicited (for instance, a canine cytokine for preparations to be administered to dogs).

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

In the case of therapeutic and/or pharmaceutical compositions based on a plasmid vector, a dose can comprise, consist essentially of or consist of, in general terms, about in 1 μg to about 2000 μg, advantageously about 50 μg to about 1000 μg and more advantageously from about 100 μg to about 800 μg of plasmid expressing the influenza antigen, epitope or immunogen. When the therapeutic and/or pharmaceutical compositions based on a plasmid vector is administered with electroporation the dose of plasmid is generally between about 0.1 μg and 1 mg, advantageously between about 1 μg and 100 μg, advantageously between about 2 μg and 50 μg. The dose volumes can be between about 0.1 and about 2 ml, advantageously between about 0.2 and about 1 ml. These doses and dose volumes are suitable for the treatment of canines and other mammalian target species such as equines and felines.

The therapeutic and/or pharmaceutical composition contains per dose from about 10⁴ to about 10¹¹, advantageously from about 10⁵ to about 10¹⁰ and more advantageously from about 10⁶ to about 10⁹ viral particles of recombinant adenovirus expressing an influenza antigen, epitope or immunogen. In the case of therapeutic and/or pharmaceutical compositions based on a poxvirus, a dose can be between about 10² pfu and about 10⁹ pfu. The pharmaceutical composition contains per dose from about 10⁵ to 10⁹, advantageously from about 10⁶ to 10⁸ pfu of poxvirus or herpesvirus recombinant expressing the influenza antigen, epitope or immunogen.

The dose volume of compositions for target species that are mammals, e.g., the dose volume of canine compositions, based on viral vectors, e.g., non-poxvirus-viral-vector-based compositions, is generally between about 0.1 to about 2.0 ml, preferably between about 0.1 to about 1.0 ml, and more preferably between about 0.5 ml to about 1.0 ml.

With inactivated compositions of the virus or organism or pathogen produced on the new cell culture, the animal may be administered approximately 10⁴-10⁹ equivalent CCID₅₀ (titer before inactivation), advantageously approximately 10⁵-10⁸ equivalent CCID₅₀ in a single dosage unit. Alternately, embryonated chicken eggs may provide a substrate for pathogen propagation, in which case the unit of measure is median egg infectious dose (EID₅₀). The volume of one single dosage unit can be between 0.2 ml and 5.0 ml and advantageously between 0.5 ml and 2.0 ml and more advantageously about 2.0 ml. One or more administrations can be done; e.g. with two injections at 2-4 weeks interval, and advantageously with a boost about 3 weeks after the first injection.

In an advantageous embodiment, an animal, advantageously a dog, is vaccinated with two doses of inactivated vaccine at about 3 to 4 week intervals via the subcutaneous route, although an intramuscular route is also contemplated. Blood samples may be collected on the day of the first and/or second vaccination and about 2 to 4 weeks after the second vaccination to determine the levels of anti-influenza virus specific antibodies by methods known to one of skill in the art, for example, virus neutralization, hemagglutination inhibition, ELISA or single radial heamolysis (SRH) tests.

The efficacy of the inactivated vaccines may be tested about 2 to 4 weeks after the second immunization by challenging animals, advantageously dogs, with a virulent strain of influenza, advantageously the influenza H3N8 strain. The animal may be challenged by spray, intra-nasally, intra-tracheally and/or orally. The challenge viral may be about 10⁵⁻⁸ EID50 in a volume depending upon the route of administration. For example, if the administration is by spray, a virus suspension is aerosolized to generate about 1 to 100 μm droplets, if the administration is intra-nasal, intra-tracheal or oral, the volume of the challenge virus is about 0.5 ml, 1-2 ml and 5-10 ml, respectively. Animals may be observed daily for 14 days following challenge for clinical signs, for example, fever, cough, nasal, ocular discharge, respiratory distress, anorexia and lethargy. In addition, groups of animals may be euthanized and evaluated for pathological findings of pulmonary and pleural hemorrhage, tracheitis, bronchitis, broncholilitis, and bronchopneumonia. Tracheal swabs may be collected from all animals post challenge days 1-14 for virus isolation. The presence or absence of viral antigens in respiratory tissues may be evaluated by immunohistochemistry, for example, on days 3, 7 and 10 post-challenge. Blood samples may be collected post-challenge (e.g., on days 7 and 14 post-challenge) and may be analyzed for the presence of anti-influenza H3N8 virus specific antibody.

It should be understood by one of skill in the art that the disclosure herein is provided by way of example and the present invention is not limited thereto. From the disclosure herein and the knowledge in the art, the skilled artisan can determine the number of administrations, the administration route, and the doses to be used for each injection protocol, without any undue experimentation.

The present invention contemplates at least one administration to an animal of an efficient amount of the therapeutic composition made according to the invention. The animal may be male, female, pregnant female and newborn. This administration may be via various routes including, but not limited to, intramuscular (IM), intradermal (ID) or subcutaneous (SC) injection or via intranasal or oral administration. The therapeutic composition according to the invention can also be administered by a needleless apparatus (as, for example with a Pigjet, Biojector or Vitajet apparatus (Bioject, Oregon, USA)). Another approach to administer plasmid compositions is to use electroporation (see, e.g. S. Tollefsen et al. Vaccine, 2002, 20, 3370-3378; S. Tollefsen et al. Scand. J. Immunol., 2003, 57, 229-238; S. Babiuk et al., Vaccine, 2002, 20, 3399-3408; PCT Application No. WO99/01158). In another embodiment, the therapeutic composition is delivered to the animal by gene gun or gold particle bombardment. In an advantageous embodiment, the animal is a vertebrate. In a more advantageous embodiment, the vertebrate is a dog.

One embodiment of the invention is a method of eliciting an immune response against influenza in an animal, comprising administering a formulation for delivery and expression of a recombinant poxvirus influenza vaccine or inactivated influenza vaccine in an effective amount for eliciting an immune response. Still another embodiment of the invention is a method of inducing an immunological or protective response against influenza in an animal, comprising administering to the animal an effective amount of a formulation for delivery and expression of an influenza antigen, epitope or immunogen wherein the formulation comprises recombinant poxvirus influenza vaccine or inactivated influenza vaccine and a pharmaceutically or veterinarily acceptable carrier, vehicle or excipient.

The invention relates to a method to elicit, induce or stimulate the immune response of an animal, advantageously a vertebrate. In one embodiment, the vertebrate is a dog but may also be a cat.

Another embodiment of the invention is a kit for performing a method of inducing an immunological or protective response against influenza in an animal comprising a recombinant influenza poxvirus vaccine or an inactivated influenza vaccine and instructions for performing the method of delivery in an effective amount for eliciting an immune response in the animal.

The invention will now be further described by way of the following non-limiting examples.

### EXAMPLE 1

### Vaccination of Dogs with Canarypox Expressing H3 Genes

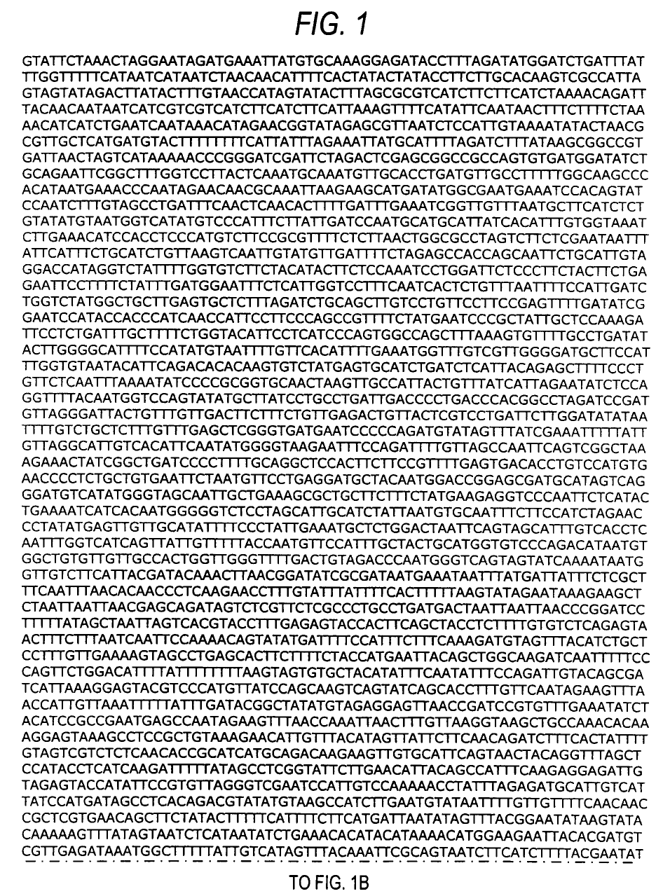

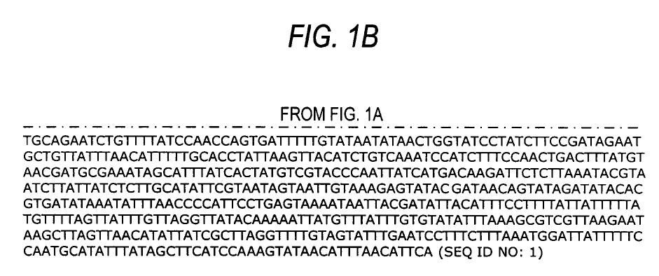

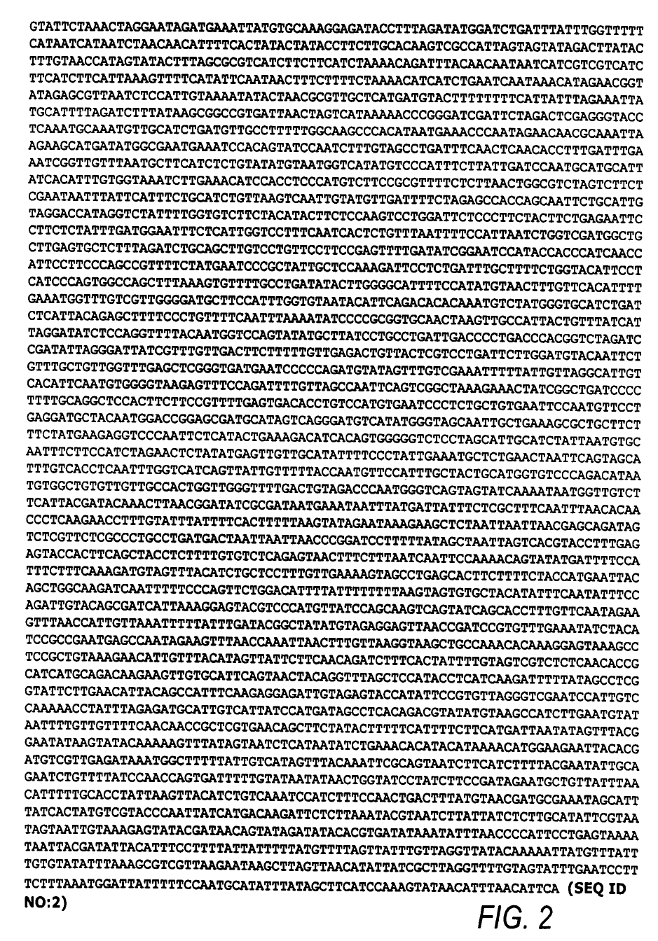

A study was conducted in which dogs were vaccinated dogs on days 0 and 21 with a canarypox expressing H3 genes from Kentucky (CP1529) and Newmarket (CP1533) equine influenza. The construction of CP1529 and CP1533 is described in Examples 1 (c5 locus), 4 and 5 of International Patent Publication WO99/44633, the disclosure of which is incorporated by reference. The nucleotide sequence of the donor plasmids pJT004 and pJT005 are presented in FIGS. 1 and 2.

Sera was collected and tested against H3N8 influenza viruses and the other viruses. As shown in Table 1, canarypox expressing hemagglutinin H3 genes induced a substantial amount of antibodies which specifically reacted with H3N8 strains but not with H1N1 or H7N7. Importantly antibodies were detectable within two weeks after the first immunization.

Accordingly, a canarypox expressing an influenza HA gene is immunogenic in dogs.

TABLE 1
Vaccination of dogs with canarypox expressing H3 hemaglutinin genes

| Antigen | Bleed | VACCINATED GROUP | | | | | CONTROL GROUP | | | | |
|---|---|---|---|---|---|---|---|---|---|---|---|
| N/1/93 (H3N8) | D0 | 0 | 0 | 0 | 0 | 0 | 0 | 0 | 0 | 0 | 0 |
| | D14 | 0 | 63.8 | 69.9 | 31.4 | 96.7 | n.s. | n.s. | n.s. | n.s. | n.s. |
| | D21 | 104.1 | 91.4 | 121.5 | 74.6 | 121.5 | n.s. | n.s. | n.s. | n.s. | n.s. |
| | D36 | 106 | 96.7 | 111.7 | 69.9 | 123.6 | n.s. | n.s. | n.s. | n.s. | n.s. |
| | D51 | 76.2 | 55.1 | 74.6 | 38.3 | 69.9 | n.s. | n.s. | n.s. | n.s. | n.s. |
| N/2/93 (H3N8) | D0 | 0 | 0 | 0 | 0 | 0 | 0 | 0 | 0 | 0 | 0 |
| | D14 | 0 | 25.1 | 59.4 | 29.2 | 60.8 | n.s. | n.s. | n.s. | n.s. | n.s. |
| | D21 | 86.2 | 71.4 | 102.2 | 65.3 | 406 | n.s. | n.s. | n.s. | n.s. | n.s. |
| | D36 | 82.8 | 74.6 | 96.7 | 62.3 | 100.4 | n.s. | n.s. | n.s. | n.s. | n.s. |
| | D51 | 52.3 | 37.1 | 66.8 | 26.1 | 57.9 | n.s. | n.s. | n.s. | n.s. | n.s. |
| Pr/56 (H7N7) | D0 | 0 | 0 | 0 | 0 | 0 | 0 | 0 | 0 | 0 | 0 |
| | D14 | 0 | 19.3 | 0 | 24.1 | 0 | n.s. | n.s. | n.s. | n.s. | n.s. |
| | D21 | 0 | 0 | 0 | 0 | 0 | n.s. | n.s. | n.s. | n.s. | n.s. |
| | D36 | 0 | 0 | 0 | 0 | 0 | n.s. | n.s. | n.s. | n.s. | n.s. |
| | D51 | 0 | 0 | 0 | 0 | 0 | n.s. | n.s. | n.s. | n.s. | n.s. |
| PR8 (H1N1) | D0 | 0 | 0 | 0 | 0 | 0 | 0 | 0 | 0 | 0 | 0 |
| | D14 | 0 | 16.7 | 0 | 19.3 | 0 | n.s. | n.s. | n.s. | n.s. | n.s. |
| | D21 | 0 | 0 | 0 | 0 | 0 | n.s. | n.s. | n.s. | n.s. | n.s. |
| | D36 | 0 | 0 | 0 | 0 | 0 | n.s. | n.s. | n.s. | n.s. | n.s. |
| | D51 | 0 | 0 | 0 | 0 | 0 | 0 | 0 | 0 | 0 | 0 |

n.s. = no sample

### EXAMPLE 2

### Contruction of the Donor Plasmid pALVAC C5 H6p-Synthetic EIV H3 HA, pJY1571.1)

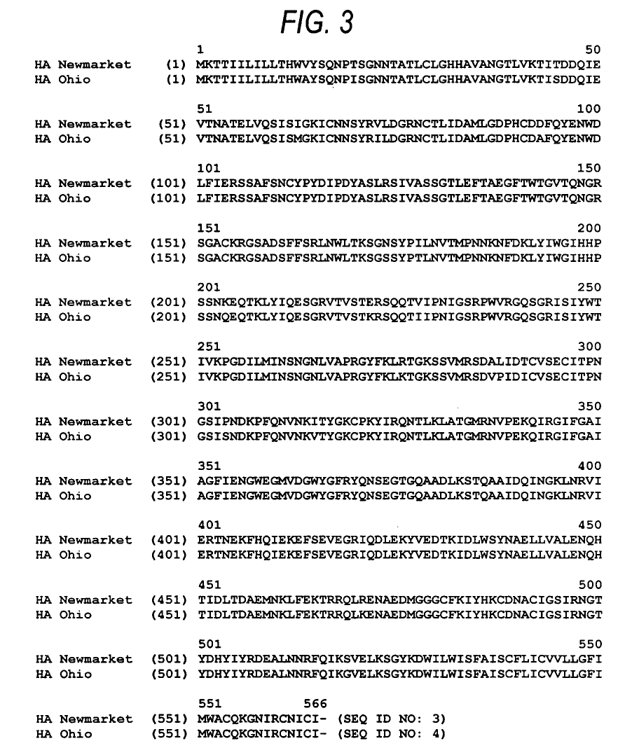

The HA gene was derived from equine influenza virus (EIV) H3N8 Ohio 03 strain isolated from a horse in 2003. The HA gene was synthetic with codon optimization for expression in mammlian cells. The amino acid sequence of EIV Ohio 03 strain HA was compared to that of New Market strain H3 HA and is presented in FIG. 3.

The purpose was the construction of an ALVAC donor plasmid for generation of an ALVAC recombinant expressing codon optimized EIV H3 HA (Ohio 03). The plasmid name was pALVAC C5 H6p-synthetic EIV H3 HA, pJY1571.1. The plasmid backbone is pALVAC C5 H6p. The promoter was a H6 promoter.

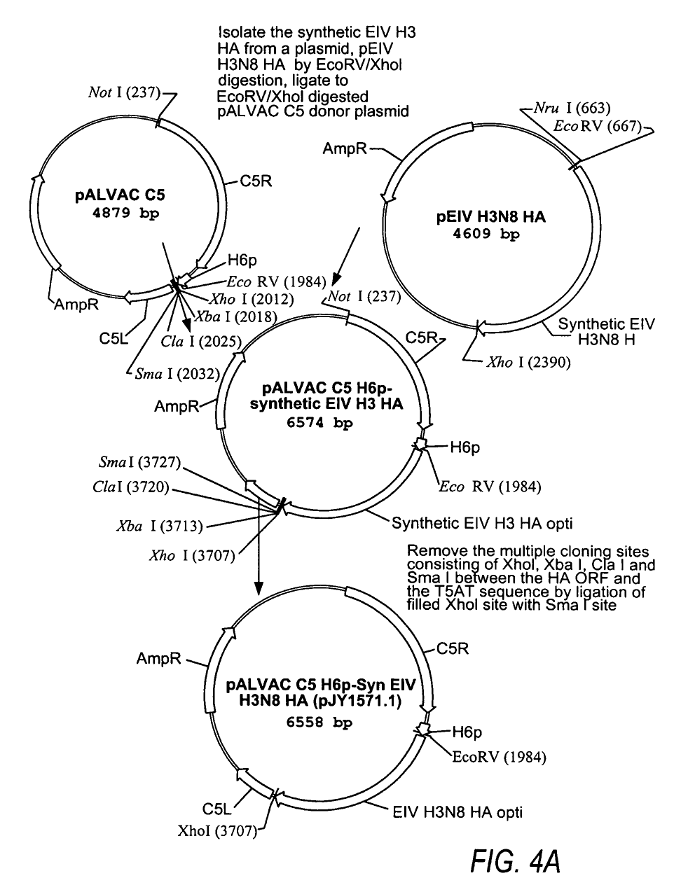

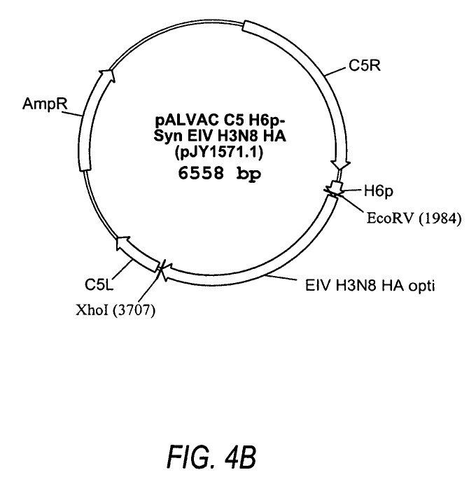

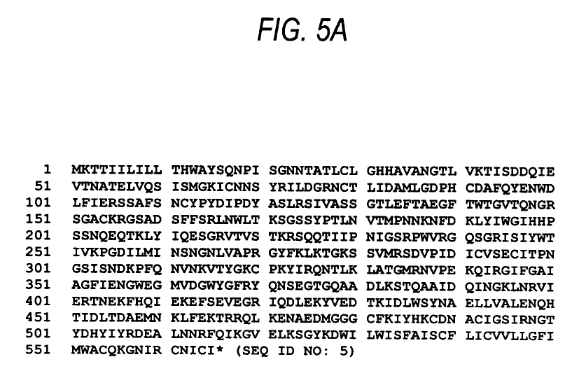

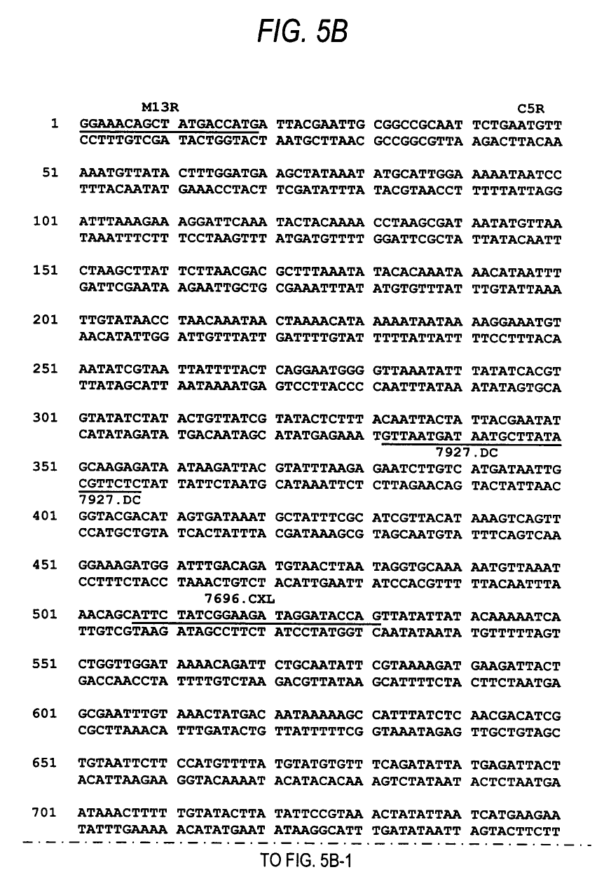

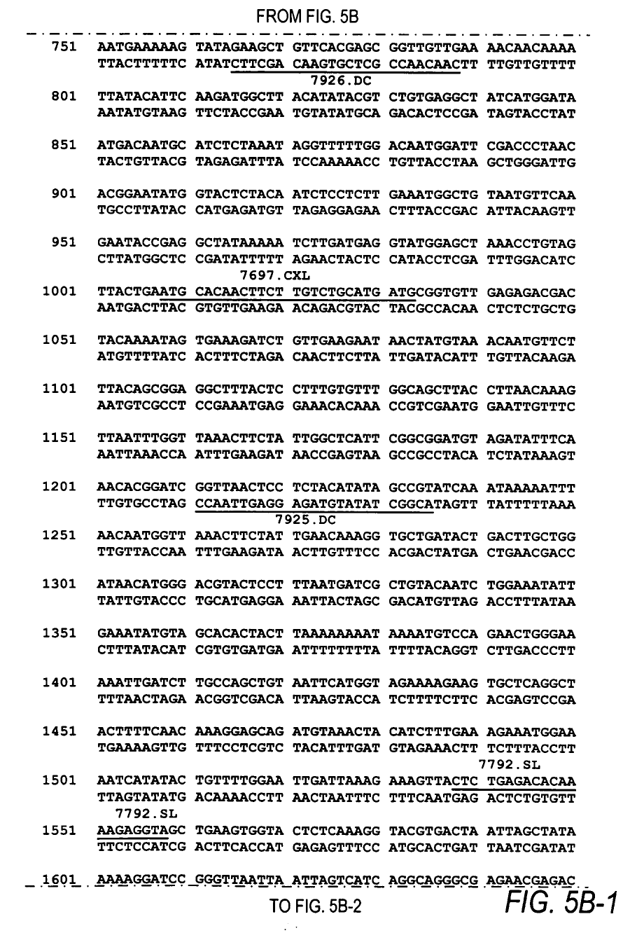

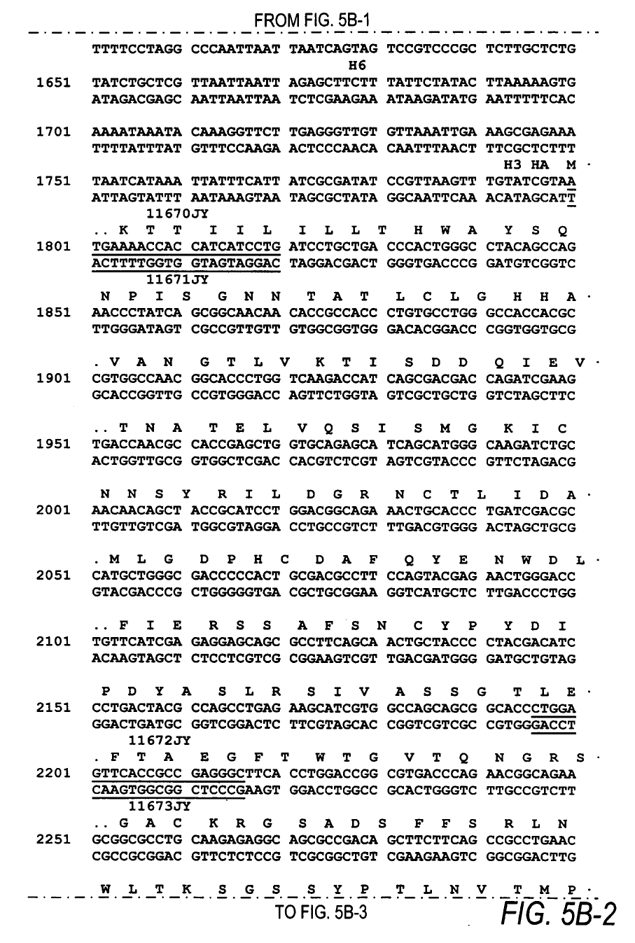

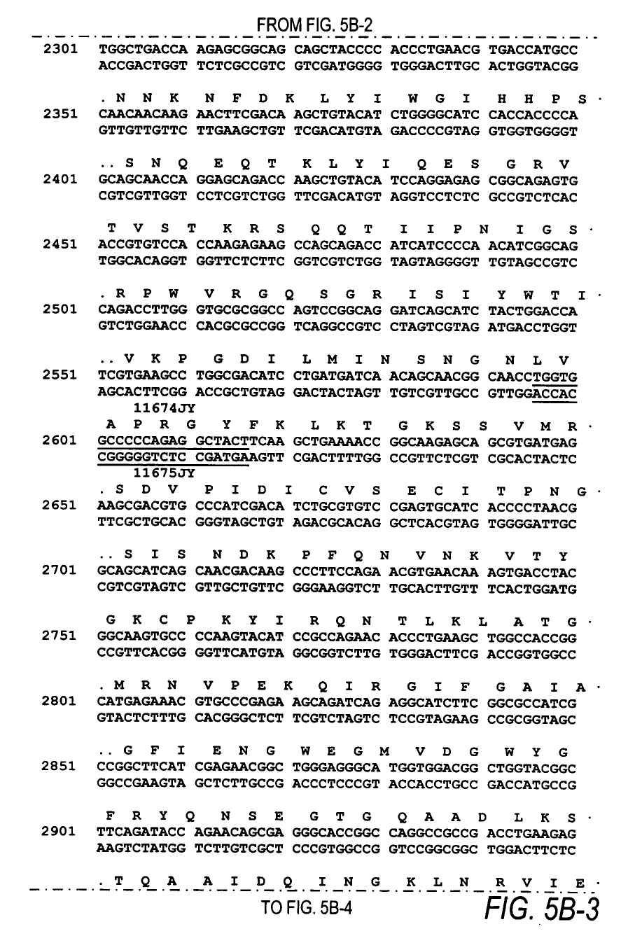

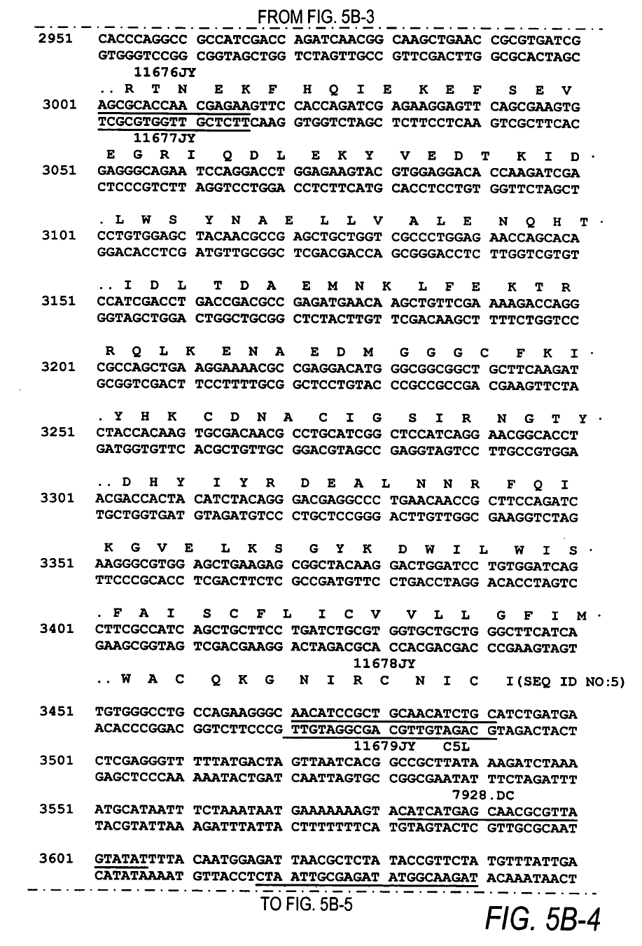

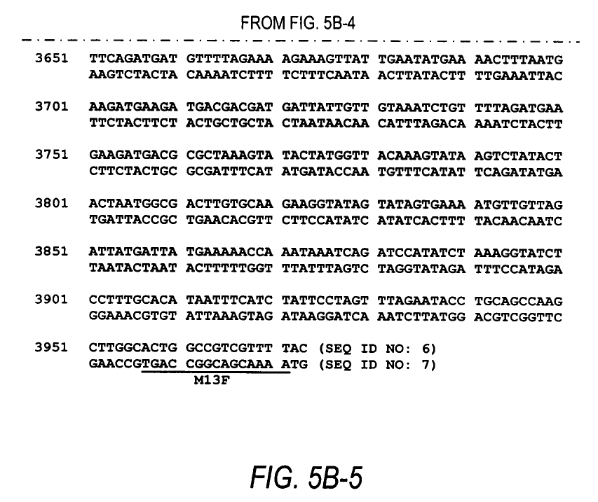

The description of plasmid construction was as follows and presented schematically in FIG. 4A. The synthetic EIV H3 HA (Ohio 03) was isolated from a plasmid pEIV H3N8 HA by EcoRV/XhoI digestion, and ligated to EcoRV/XhoI digested donor plasmid pALVAC C5 H6p to create pALVAC C5 H6p-Synthetic EIV H3 HA. In the resulting plasmid, there are multiple cloning sites consisting of XhoI, Xba I, Cla I and Sma I between the HA ORF and the T5AT sequence which serves as the transcription termination signal. To bring the HA ORF and the T5AT sequence close together, those cloning sites were then subsequently removed by ligation of re-filled XhoI site with Sma I site. The resulting plasmid pJY1571.1 was then sequenced and confirmed to contain the correct sequence. A diagram of the resulting plasmid is presented in FIG. 4B. The predicted amino acid sequence of EIV H3 HA is shown in FIG. 5A and the nucleotide sequence of arms and insert with translation is shown in FIG. 5B.

### EXAMPLE 3

### Construction of the Recombinant Canarypox vCP2242

The purpose of this example was the generation and characterization of ALVAC recombinant containing EIV H3N8 codon optimized HA inserted at C5 loci of ALVAC (vCP2242). The parental virus was ALVAC, the donor plasmid was pJY1571.1, the insertion site was a C5 Locus, the promoter was a H6 promoter and cells for in vitro recombination were primary chicken embryo fibroblast cells (1° CEF).

The in vitro recombination (IVR) was performed by transfection of 1° CEF cells with 15 μg of Not I-linearized donor plasmid pJY1571.1. The transfected cells were subsequently infected with ALVAC as rescue virus at MOI of 10. After 24 hr, the transfected-infected cells were harvested, sonicated and used for recombinant virus screening.

Recombinant plaques were screened based on the plaque lift hybridization method using a 821 bp EIV syn HA specific probe labeled with horse radish peroxidase (HRP) according to the manufacturer's protocol. After four sequential rounds of plaque purification, the recombinant designated as vCP2242 was generated and confirmed by hybridization as 100% positive for the EIV syn HA insert and 100% negative for the C5 ORF.

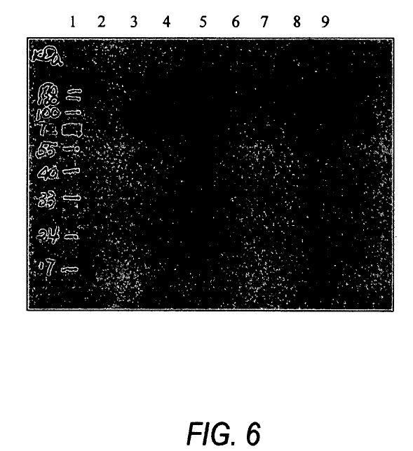

Expression analysis and sequence analysis were performed. Expression analysis was performed by Western blot. Primary CEF cells were infected with vCP2242 stock at MOI of 10 and incubated at 37 C for 26.5 hrs. The cells and culture supernatant were then harvested. Sample proteins were separated on a 10% SDS-PAGE gel, transferred to Immobilon nylon membrane, and probed with a pool of monoclonal mouse anti-EIV HA antibodies (anti Eq/AK/91: 124-ID9-1, 124-3E3-3, 124-4F3-2 and H3N8 A Eq/miami/63 pool at 1/1,000 dilution). Peroxidase-conjugated goat anti-mouse antiserum was used as a secondary antibody and the bands were visualized using luminol reagents. vCP2242 showed a protein expression profile with a 80 kDa protein expressed in cell pellet, but not in the culture medium as presented in FIG. 6.

Results of the sequence analysis demonstrated that the sequences of the EIV syn HA and C5L and C5R of ALVAC were correct.

### EXAMPLE 4

### Kentucky Equine Influenza Nucleotide Sequences

DEFINITION Influenza A virus (A/equine/Kentucky/5/02(H3N8)) nonstructural protein gene, complete cds.

```
ACCESSION AY855345; SEQ ID NO: 8
    1 agcaaaagca gggtgacaaa aacataatgg attccaacac tgtgtcaagc tttcaggtag
  61 actgttttct ttggcatgtc cgcaaacgat tcgcagacca agaactgggt gatgccccat
121 tccttgaccg gcttcgccga gaccagaagt ccctaagggg aagaggtatc actcttggtc
181 tggacatcga aacagccact catgcaggaa agcagatagt ggagcagatt ctggaaaagg
241 aatcagatga ggcacttaaa atgaccattg cctctgttcc tacttcacgc tacttaactg
301 acatgactct tgatgagatg tcaagagact ggttcatgct catgcccaag caaaaagtaa
361 caggctccct atgtataaga atggaccagg caatcatgga taagaacatc atacttaaag
421 caaactttag tgtgattttc gaaaggctgg aaacactaat actacttaga gccttcaccg
481 aagaaggagc agtcgttggc gaaatttcac cattaccttc tcttccagga catactaatg
541 aggatgtcaa aaatgcaatt ggggtcctca tcggaggact taaatggaat gataatacgg
601 ttagaatctc tgaaactcta cagagattcg cttggagaag cagtcatgag aatgggagac
661 cttcattccc ttcaaagcag aaatgaaaaa tggagagaac aattaagcca gaaatttgaa
721 gaaataagat ggttgattga agaagtgcga catagattga aaaatacaga aaatagtttt
781 gaacaaataa catttatgca agccttacaa ctattgcttg aagtagaaca agagataaga
841 actttctcgt ttcagcttat ttaatgataa aaaacaccct tgtttctact
```

DEFINITION Influenza A virus (A/equine/Kentucky/5/02(H3N8)) matrix protein gene, complete cds.

```
ACCESSION AY855344; SEQ ID NO: 9
      1 agcaaaagca ggtagatatt taaagatgag tcttctgacc gaggtcgaaa cgtacgttct
    61 ctctatcgta ccatcaggcc ccctcaaagc cgagatcgcg cagagacttg aagatgtctt
  121 tgcagggaag aacaccgatc ttgaggcact catggaatgg ctaaagacaa gaccaatcct
  181 gtcacctttg actaaaggga ttttaggatt tgtattcacg ctcaccgtgc ccagtgagcg
  241 aggactgcag cgtagacgct ttgtccaaaa tgcccttagt ggaaacggag atccaaacaa
  301 catggacaga gcagtaaaac tgtacaggaa gcttaaaaga gaaataacat tccatggggc
  361 aaaagaggtg gcactaagct attccactgg tgcactagcc agctgcatgg gactcatata
  421 caacagaatg ggaactgtga caaccgaagt ggcatttggc ctggtatgcg ccacatgtga
  481 acagattgct gattcccagc atcgatctca caggcagatg gtgacaacaa ccaacccatt
  541 aatcagacat gaaaacagaa tggtattagc cagtaccacg gctaaagcca tggaacagat
  601 ggcaggatca agtgagcagg cagcagaggc catggaggtt gctagtaagg ctaggcagat
  661 ggtacaggca atgagaacca ttgggaccca ccctagctcc agtgccggtt tgaaagatga
  721 tctccttgaa aatttacagg cctaccagaa acggatggga gtgcaaatgc agcgattcaa
  781 gtgatcctct cgttattgca gcaagtatca ttgggatctt gcacttgata ttgtggattc
  841 ttgatcgtct tttcttcaaa ttcatttatc gtcgccttaa atacgggttg aaaagagggc
  901 cttctacgga aggagtacct gagtctatga gggaagaata tcggcaggaa cagcagaatg
  961 ctgtggatgt tgacgatggt cattttgtca acatagagct ggagtaaaaa actaccttgt
1021 ttctact
```

DEFINITION Influenza A virus (A/equine/Kentucky/5/02(H3N8)) neuraminidase gene, complete cds.

```
ACCESSION AY855343; SEQ ID NO: 10
      1 agcaaaagca ggagtttaaa atgaatccaa atcaaaagat aatagcaatt ggatttgcat
    61 cattggggat attaatcatt aacgtcattc tccatgtagt cagcattata gtaacagtac
  121 tggtcctcaa taacaatgga acaggtctga actgcaaagg gacgatcata agagagtaca
  181 atgaaacagt aagagtagaa aaaattactc aatggtataa taccagtgca attaagtaca
  241 tagagagacc tccaaatgaa tactacatga acaacaccga accactttgt gaggcccaag
  301 gctttgcacc attttccaaa gataatggaa tacgaattgg gtcgagaggc catgtttttg
  361 tgataagaga accttttgta tcatgttcgc cctcagaatg tagaaccttt ttcctcacac
  421 agggctcatt actcaatgac aaacattcta acggcacagt aaaggaccga agtccatata
  481 ggactttgat gagtgtcaaa atagggcaat cacctaatgt gtatcaagct aggtttgaat
  541 cggtggcatg gtcagcaaca gcatgccatg atggaaaaaa atggatgaca gttggagtca
  601 cagggcccga caatcaagca attgcagtag tgaactatgg aggtgttccg gttgatatta
  661 ttaattcatg ggcaggggat atcttaagaa cccaagaatc atcatgcacc tgcattaaag
  721 gagactgtta ttgggtaatg actgatggac cggcaaatag gcaagctaaa tataggatat
  781 tcaaagcaaa agatggaaga gtaattggac agactgatat aagtttcaat gggggacaca
  841 tagaggagtg ttcttgttac cccaatgaag ggaaggtgga atgcatatgc agggacaatt
  901 ggactggaac aaatagacca attctggtaa tatcttctga tctatcgtac acagttggat
  961 atttgtgtgc tggcattccc actgacactc ctaggggaga ggatagtcaa ttcacaggct
1021 catgtacaag acctttggga aataaaggat acggtgtaaa aggtttcggg tttcgacaag
1081 gaactgacgt atgggccgga aggacaatta gtaggacttc aagatcagga ttcgaaataa
1141 taaaaatcag gaatggttgg acacagaaca gtaaagacca aatcaggagg caagtgatta
1201 tcgatgaccc aaattggtca ggatatagcg gttctttcac attgccggtt gaactaacaa
1261 aaaagggatg tttggtcccc tgtttctggg ttgaaatgat tagaggtaaa cctgaagaaa
1321 caacaatatg gacctctagc agctccattg tgatgtgtgg agtagatcat aaaattgcca
1381 gttggtcatg gcacgatgga gctattcttc cctttgacat cgataagatg taatttacga
1441 aaaaactcct tgtttctact
```

DEFINITION Influenza A virus (A/equine/Kentucky/5/02(H3N8)) nucleoprotein gene, complete cds.

```
ACCESSION AY855342; SEQ ID NO: 11
      1 agcaaaagca gggtagataa tcactcactg agtgacatca aagtcatggc gtctcaaggc
    61 accaaacgat cctatgaaca gatggaaact gatggggaac gccagaatgc aactgaaatc
  121 agagcatctg tcggaaggat ggtgggagga atcggccggt tttatgttca gatgtgtact
  181 gagcttaaac taaacgacca tgaagggcgg ctgattcaga acagcataac aatagaaagg
  241 atggtacttt cggcattcga cgaaagaaga aacaagtatc tcgaggagca tcccagtgct
  301 ggaaaagacc ctaagaaaac gggaggcccg atatacagaa ggaaagatgg gaaatggatg
  361 agggaactca tcctccatga taaagaagaa atcatgagaa tctggcgtca ggccaacaat
  421 ggtgaagacg ctactgctgg tcttactcat atgatgatct ggcactccaa tctcaatgac
  481 accacatacc aaagaacaag ggctcttgtt cggactggga tggatcccag aatgtgctct
  541 ctgatgcaag gctcaaccct cccacggaga tctggagccg ctggtgctgc agtaaaaggt
  601 gttggaacaa tggtaatgga actcatcaga atgatcaaac gcggaataaa tgatcggaat
  661 ttctggagag gtgaaaatgg tcgaaggacc agaattgctt atgaaagaat gtgcaatatc
  721 ctcaaaggga aatttcagac agcagcacaa cgggctatga tggaccaggt gagggaaggc
  781 cgcaatcctg gaaacgctga gattgaggat ctcattttct tagcacgatc agcacttatt
  841 ttgagaggat cagtagccca taaatcatgc ctacctgcct gtgtttatgg ccttgcagta
  901 accagtgggt atgactttga gaaggaagga tactctctgg ttggaattga tcctttcaaa
  961 ctactccaga acagtcaaat tttcagtcta atcagaccaa aagaaaaccc agcacacaag
1021 agccagttgg tgtggatggc atgccattct gcagcatttg aggacctgag agttttgaat
1081 ttcattagag gaaccaaagt aatcccaaga ggacagttaa caaccagagg agttcaaatt
1141 gcttcaaatg aaaacatgga gacaatagat tctagcacac ttgaactgag aagcaaatat
1201 tgggcaataa ggaccagaag tggaggaaac accagtcaac agagagcatc tgcaggacag
1261 ataagtgtgc aacctacttt ctcagtacag agaaatcttc cctttgagag agcaaccatt
1321 atggctgcat tcactggtaa cactgaaggg aggacttccg acatgagaac ggaaatcata
1381 aggatgatgg aaagtgccaa atcagaagat gtgtctttcc aggggcgggg agtcttcgag
1441 ctctcggacg aaaaggcaac gaacccgatc gtgccttcct ttgacatgag caatgaaggg
1501 tcttatttct tcggagacaa tgctgaggaa tttgacagtt aaagaaaaat acccttgttt
1561 ctact
```

DEFINITION Influenza A virus (A/equine/Kentucky/5/02(H3N8)) hemagglutinin precursor, gene, complete cds.

```
ACCESSION AY855341; SEQ ID NO: 12
      1 agcaaaagca ggggatattt ctgtcaatca tgaagacaac cattattttg atactactga
    61 cccattgggc ctacagtcaa aacccaatca gtggcaacaa cacagccaca ttgtgtctgg
  121 gacaccatgc agtagcaaat ggaacattgg taaaaacaat aagtgatgat caaattgagg
  181 tgacaaatgc tacagaatta gttcagagca tttcaatggg gaaaatatgc aacaactcat
  241 atagaattct agatggaaga aattgcacat taatagatgc aatgctagga gacccccact
  301 gtgacgtctt tcagtatgag aattgggacc tctttataga aagaagcagc gctttcagca
  361 attgctaccc atatgacatc cctgactatg catcgctccg atccattgta gcatcctcag
  421 gaacattgga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 agctatacat ctgggggatt catcacccga gctcaaatca agagcagaca aaattgtaca
  661 tccaagaatc aggacgagta acagtctcaa caaaaagaag tcaacaaaca ataatcccta
  721 acatcggatc tagaccgtgg gtcagaggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatctc caacgacaag ccattccaaa
  961 atgtgaacaa agttacatat ggaaaatgcc ccaagtatat caggcaaaac actttaaagt
1021 tggccactgg gatgaggaat gtaccagaaa agcaaatcag aggaatcttt ggagcaatag
1081 cgggattcat cgaaaacggc tgggaaggaa tggttgatgg gtggtatggg ttccgatatc
1141 aaaactctga aggaacaggg caagctgcag atctaaagag cactcaagca gccatcgacc
1201 agattaatgg aaagttaaac agagtgattg aaagaaccaa tgagaaattc catcaaatag
1261 agaaggaatt ctcagaagta gaaggaagaa ttcaggactt ggagaaatat gtagaagaca
1321 ccaaaataga cctatggtcc tacaatgcag aattgctggt ggctctagaa aatcaacata
1381 caattgactt aacagatgca gaaatgaata aattattcga gaagactaga cgccagttaa
1441 gagaaaacgc agaagacatg ggaggtggat gtttcaagat ttaccacaaa tgtgataatg
1501 catgcattgg atcaataaga aatgggacat atgaccatta catatacaga gatgaagcat
1561 taaacaaccg atttcaaatc aaaggtgttg agttgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A virus (A/equine/Kentucky/5/02(H3N8)) PA polymerase gene, complete cds.

```
ACCESSION AY855340; SEQ ID NO: 13
      1 agcgaaagca ggtactgatc caaaatggaa gactttgtgc gacagtgctt caatccaatg
    61 atcgtcgagc ttgcggaaaa ggcaatgaaa gaatatggag aggacccgaa aatcgaaaca
  121 aacaaatttg cagcaatatg cactcacttg gaagtctgct tcatgtactc ggatttccac
  181 tttattaatg aactgggtga gtcagtggtc atagagtctg gtgacccaaa tgctcttttg
  241 aaacacagat ttgaaatcat tgaggggaga gatcgaacaa tggcatggac agtggtaaac
  301 agcatctgca acaccacaag agctgaaaaa cctaaatttc ttccagattt atacgactat
  361 aaggagaaca gatttgttga aattggtgtg acaaggagag aagttcacat atactacctg
  421 gagaaggcca acaaaataaa gtctgagaaa acacatatcc acattttctc atttacagga
  481 gaggaaatgg ctacaaaagc ggactatact cttgatgaag agagtagagc caggatcaag
  541 accagactat tcactataag acaagaaatg gccagtagag gcctctggga ttcctttcgt
  601 cagtccgaga gaggcgaaga gacaattgaa gaaagatttg aaatcacagg gacgatgcgc
  661 aagcttgcca attacagtct cccaccgaac ttctccagcc ttgaaaattt tagagtctat
  721 gtggatggat tcgaaccgaa cggctgcatt gagagtaagc tttctcaaat gtccaaagaa
  781 gtaaatgcca gaatcgaacc attttcaaag acaacacccc gaccactcaa aatgccaggt
  841 ggtccaccct gccatcagcg atctaaattc ttgctaatgg atgctctgaa actgagcatt
  901 gaggacccaa gtcacgaggg agagggaata ccactatatg atgcaatcaa atgcatgaaa
  961 actttctttg gatggaaaga gcccagtatt gttaaaccac atgaaaaggg tataaacccg
1021 aactatctcc aaacttggaa gcaagtatta gaagaaatac aagaccttga gaacgaagaa
1081 aggaccccca agaccaagaa tatgaaaaaa acaagccaat tgaaatgggc actaggtgaa
1141 aatatggcac cagagaaagt ggattttgag gattgtaaag acatcagtga tttaaaacag
1201 tatgacagcg atgagccaga aacaaggtct cttgcaagtt ggattcaaag tgagttcaac
1261 aaagcttgtg agctgacaga ttcaagctgg atagagctcg atgaaattgg ggaggatgtc
1321 gccccaatag aatacattgc gagcatgagg agaaattatt ttactgctga gatttcccat
1381 tgtagagcaa cagaatatat aatgaaagga gtgtacatca acactgctct actcaatgca
1441 tcctgtgctg cgatggatga atttcaatta attccgatga taagtaaatg caggaccaaa
1501 gaagggagaa ggaaaacaaa tttatatgga ttcataataa agggaaggtc ccatttaaga
1561 aatgatactg acgtggtgaa ctttgtaagt atggaatttt ctctcactga tccaagattt
1621 gagccacaca aatgggaaaa atactgcgtt ctagaaattg gagacatgct tctaaggact
1681 gctgtaggtc aagtgtcaag acccatgttt ttgtatgtaa ggacaaatgg aacctctaaa
1741 attaaaatga aatggggaat ggaaatgagg cgctgcctcc ttcagtctct gcaacagatt
1801 gaaagcatga tcgaagctga gtcctcagtc aaagaaaagg acatgaccaa agaatttttt
1861 gagaacaaat cagagacatg gcctatagga gagtccccca aaggagtgga agagggctca
1921 atcgggaagg tttgcaggac cttattagca aaatctgtgt ttaacagttt atatgcatct
1981 ccacaactgg aagggttttc agctgaatct aggaaattac ttctcattgt tcaggctctt
2041 agggataacc tggaacctgg aacctttgat attggggggt tatatgaatc aattgaggag
2101 tgcctgatta atgatccctg ggttttgctt aatgcatctt ggttcaactc cttccttaca
2161 catgcactga agtagttgtg gcaatgctac tatttgctat ccatactgtc caaaaaagta
2221 ccttgtttct act
```

DEFINITION Influenza A virus (A/equine/Kentucky/5/02(H3N8)) PB1 polymerase 1 gene, complete cds.

```
ACCESSION AY855339; SEQ ID NO: 14
      1 agcgaaagca ggcaaaccat ttgaatggat gtcaatccga ctctactttt cttaaaggtg
    61 ccagcgcaaa atgctataag cacaacattc ccttatactg gagatcctcc ctacagtcat
  121 ggaacaggga caggatacac catggatact gtcaacagaa cacaccaata ttcagaaaag
  181 gggaaatgga caacaaacac tgagattgga gcaccacaac ttaatccaat cgatggacca
  241 cttcctgaag acaatgaacc aagtgggtac gcccaaacag attgtgtatt ggaagcaatg
  301 gctttccttg aagaatccca tcccggaatc tttgaaaatt cgtgtcttga aacgatggag
  361 gtgattcagc agacaagagt ggacaaacta acacaaggcc gacaaactta tgattggacc
  421 ttgaatagga atcaacctgc cgcaacagca cttgctaata caattgaagt gttcagatca
  481 aatggtctga cttccaatga atcagggagg ttgatggact tcctcaaaga tgtcatggag
  541 tccatgaaca aggaagaaat ggaaataaca acacacttcc aacgaaagag aagagtaaga
  601 gacaacatga caaagagaat ggtaacacag agaaccatag ggaagaaaaa acaacgatta
  661 aacagaaaga gttatctaat cagaacatta accctaaaca caatgaccaa ggacgctgag
  721 agagggaaat tgaaacgacg agcaatcgca accccaggga tgcagataag aggatttgta
  781 tattttgttg aaacactagc ccgaagaata tgtgaaaagc ttgaacaatc aggattgcca
  841 gttggcggta atgagaaaaa ggccaaactg gctaatgtcg tcagaaaaat gatgactaat
  901 tcccaagaca ctgaactctc cttcaccatc actggggaca ataccaaatg gaatgaaaat
  961 cagaacccac gcatattcct ggcaatgatc acatacataa ctagaaacca gccagaatgg
1021 ttcagaaatg ttctaagcat tgcaccgatt atgttctcaa ataaaatggc aagactgggg
1081 aaaggatata tgtttgaaag caaaagtatg aaactgagag ctcaaatacc agcagaaatg
1141 ctagcaagca ttgacctgaa atatttcaat gattcaacaa aaaagaaaat taaaaagata
1201 cgaccacttc tggttgacgg gactgcttca ctgagtcctg gcatgatgat gggaatgttc
1261 aacatgttga gcactgtgct gggtgtatcc atattaaacc tgggccagag gaaatacaca
1321 aagaccacat actggtggga tggtctgcaa tcatccgatg actttgcttt gatagtgaat
1381 gcgcctaatc atgaaggaat acaagctgga gtagacagat tctatagaac ttgcaaactg
1441 gtcgggatca acatgagcaa aaagaagtcc tacataaata gaaccggaac attcgaattc
1501 acaagctttt tctaccggta tggttttgta gccaatttca gcatggaact acccagtttt
1561 ggggtttccg gaataaatga atctgcagac atgagcattg gagtgacagt catcaaaaac
1621 aacatgataa ataatgatct cggtcctgcc acggcacaaa tggcactcca actcttcatt
1681 aaggattacc ggtacacata ccggtgccat agaggtgata cccagataca aaccagaaga
1741 tcttttgagt tgaagaaact gtgggaacag actcgatcaa agactggtct actggtatca
1801 gatgggggtc caaacctata taacatcaga aacctacaca tcccggaagt ttgtttaaaa
1861 tgggagctaa tggatgaaga ttataagggg aggctatgca atccattaaa tcctttcgtt
1921 agtcacaaag aaattgaatc agtcaacagt gcagtagtaa tgcctgcgca tggccctgcc
1981 aaaagcatgg agtatgatgc tgttgcaaca acacattctt ggatccccaa gaggaaccgg
2041 tccatattga acacaagcca aaggggaata ctcgaagatg agcagatgta tcagaaatgc
2101 tgcaacctgt ttgaaaaatt cttccccagc agctcataca gaagaccagt cggaatttct
2161 agtatggttg aggccatggt gtccagggcc cgcattgatg cacgaattga cttcgaatct
2221 ggacggataa agaaggatga gttcgctgag atcatgaaga tctgttccac cattgaagag
2281 ctcagacggc aaaaatagtg aatttagctt gatcttcatg aaaaaatgcc ttgtttctac
2341 t
```

DEFINITION Influenza A virus (A/equine/Kentucky/5/02(H3N8)) PB2 polymerase 2 gene, complete cds.

```
ACCESSION AY855338; SEQ ID NO: 15
      1 agcgaaagca ggtcaaatat attcaatatg gagagaataa aagaactgag agatctgatg
    61 ttacaatccc gcacccgcga gatactaaca aaaactactg tggaccacat ggccataatc
  121 aagaaataca catcaggaag acaagagaag aaccctgcac ttaggatgaa atggatgatg
  181 gcaatgaaat acccaattac agcagataag aggataatgg agatgattcc tgagagaaat
  241 gaacagggac aaaccctttg gagcaaaacg aacgatgctg gctcagaccg cgtaatggta
  301 tcacctctgg cagtgacatg gtggaatagg aatggaccaa caacgagcac aattcattat
  361 ccaaaagtct acaaaactta ttttgaaaag gttgaaagat tgaaacacgg aacctttggc
  421 cccgttcatt ttaggaatca agtcaagata agacgaagag ttgatgtaaa ccctggtcac
  481 gcggacctca gtgccaaaga agcacaagat gtgatcatgg aagttgtttt cccaaatgaa
  541 gtgggagcca gaattctaac atcggaatca caactaacaa taaccaaaga gaaaaaggaa
  601 gaacttcagg actgcaaaat tgctcccttg atggtagcat acatgctaga aagagagttg
  661 gtccgaaaaa caaggttcct cccagtggca ggcggaacaa gcagtgtata cattgaagtg
  721 ttgcatctga ctcagggaac atgctgggag caaatgtaca ccccaggagg agaagttaga
  781 aacgatgata ttgatcaaag tttaattatt gcagcccgga acatagtgag aagagcgaca
  841 gtatcagcag atccactagc atccctactg gaaatgtgcc acagtacaca gattggtgga
  901 ataaggatgg tagacatcct taagcagaat ccaacagagg aacaagctgt ggatatatgc
  961 aaagcagcaa tgggattgag aattagctca tcattcagct ttggtggatt caccttcaaa
1021 agaacaagtg gatcatcagt caagagagaa gaagaaatgc ttacgggcaa ccttcaaaca
1081 ttgaaaataa gagtgcatga gggctatgaa gaattcacaa tggtcggaag aagagcaaca
1141 gccattctca gaaaggcaac cagaagattg attcaattga tagtaagtgg gagagatgaa
1201 caatcaattg ctgaagcaat aattgtagcc atggtgtttt cgcaagaaga ttgcatgata
1261 aaagcagttc gaggcgattt gaactttgtt aatagagcaa atcagcgttt gaaccccatg
1321 catcaactct tgaggcattt ccaaaaagat gcaaaagtgc ttttccaaaa ttggggaatt
1381 gaacccatcg acaatgtaat gggaatgatt ggaatattgc ctgacatgac cccaagcacc
1441 gagatgtcat tgagaggagt gagagtcagc aaaatgggag tggatgagta ctccagcact
1501 gagagagtgg tggtgagcat tgaccgtttt ttaagagttc gggatcaaag gggaaacata
1561 ctactgtccc ctgaagaagt cagtgaaaca caaggaacgg aaaagctgac aataatttat
1621 tcgtcatcaa tgatgtggga gattaatggt cccgaatcag tgttggtcaa tacttatcaa
1681 tggatcatca ggaactggga aattgtaaaa attcagtggt cacaggaccc cacaatgtta
1741 tacaataaga tagaatttga gccattccag tccctggtcc ctagggccac cagaagccaa
1801 tacagcggtt tcgtaagaac cctgtttcag caaatgcgag atgtacttgg aacatttgat
1861 actgctcaaa taataaaact cctccctttt gccgctgctc ctccggaaca gagtaggatg
1921 cagttctctt ctttgactgt taatgtaaga ggatcgggaa tgaggatact tgtaagaggc
1981 aattccccag tgttcaacta caataaagcc actaaaaggc tcacagtcct cggaaaggat
2041 gcaggtgcgc ttactgagga cccagatgaa ggtacggctg gagtagaatc tgctgttcta
2101 agagggtttc tcattttagg taaagaaaac aagagatatg gcccagcact aagcatcaat
2161 gaactaagca aacttgcaaa aggggagaaa gccaatgtac taattgggca aggggacgta
2221 gtgttggtaa tgaaacggaa acgtgactct agcatactta ctgacagcca gacagcgacc
2281 aaaaggattc ggatggccat caattagtgt tgaattgttt aaaaacgacc ttgtttctac
2341 t
```

DEFINITION Influenza A virus (A/equine/Kentucky/1/91(H3N8)) hemagglutinin precursor (HA) gene, complete cds.

```
ACCESSION L39918; SEQ ID NO: 16
      1 agcaaaagca ggggatattt ctgtcaatca tgaagacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagagttct agatggaaga aattgcacat taatagatgc aatgctagga gacccccact
  301 gtgatgtctt tcagtatgag aattgggacc tcttcataga aagaagcagc gctttcagca
  361 gttgctaccc atatgacatc cctgactatg catcgctccg gtccattgta gcatcctcag
  421 gaacattgga attcacagca gagggattca catggacagg tgtcactcaa aacggaagaa
  481 gtggatcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacga agagcagaca aaattgtaca
  661 tccaagaatc aggacgagta acagtctcaa caaaaagaag tcaacaaaca ataatcccta
  721 acatcggatc tagaccgtgg gtcaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaactcgga aggaacagga caagctgcag atctaaagag cactcaagca gccatcgacc
1201 agatcaatgg aaaattaaac agagtgattg aaaggaccaa tgagaaattc catcaaatag
1261 agaaggaatt ctcagaagta gaagggagaa tccaggattt ggagaagtat gtagaagaca
1321 ccaaaataga cctatggtcc tacaatgcag aattgctggt ggctctagaa aatcaacata
1381 caattgactt aacagatgca gaaatgaata aattattcga gaagactaga cgccagttaa
1441 gagaaaacgc ggaagacatg ggaggtggat gtttcaagat ataccacaaa tgtgataatg
1501 catgcattgg atcaataaga aatgggacat atgaccatta catatacaga gatgaagcat
1561 taaacaaccg gtttcaaatc aaaggtgttg agttgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A virus (A/equine/Kentucky/1/92(H3N8)) hemagglutinin precursor (HA) gene, complete cds.

```
ACCESSION L39917; SEQ ID NO: 17
      1 agcaaaagca ggggatattt ctgtcaatca tgaagacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagggttct agatggaaga aattgcacat taatagatgc aatgctagga gacccccact
  301 gtgatgtctt tcagtatgag aattgggacc tcttcataga aagaagcagc gctttcagca
  361 attgctaccc atatgacatc cctgactatg catcgctccg gtccattgta gcatcctcag
  421 gaacattaga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacaa tgagcagaca aaattgtata
  661 tccaagaaac aggacgagta acagtctcaa caaaaagaag tcaacaaaca ataatcccta
  721 acatcggatc tagaccgtgg gtcaggggtc aatcaggcag gataagcata tactggacca
  781 ttgtaaaacc tggagatatc ctaatgataa acagcaatgg caacttagtt gcaccgcggg
  841 gatattttaa attgagaaca gggagaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaaatatat caggcaaaac actttaaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaactcgga aggaacagga caagctgcag atctaaagag cactcaagca gccatcgacc
1201 agatcaatgg aaaattaaac agagtgattg aaaggaccaa tgagaaattc catcaaatag
1261 agaaggaatt ctcagaagta gaagggagaa tccaggattt ggagaagtat gtagaagaca
1321 ccaaaataga cctatggtcc tacaatgcag aattgctagt ggctctagaa aatcaacata
1381 caattgactt aacagacgca gaaatgaata aattattcga gaagactaga cgccagttaa
1441 gagaaaacgc ggaagacatg ggaggtggat gtttcaagat ttaccacaaa tgtgataatg
1501 catgcattgg atcaataaga aatgggacat atgaccatta catatacaga gatgaagcat
1561 taaacaaccg atttcaaatc aaaggtgttg aattaaaatc aggctacaaa gattggatac
1621 tgtggatttc attcgccata tcatgcttct taatttgtgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A virus (A/equine/Kentucky/1/90(H3N8)) hemagglutinin precursor (HA) gene, complete cds.

```
ACCESSION L39915; SEQ ID NO: 18
      1 agcaaaagca ggggatattt ctgtcaatca tgaaaacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg aaaaatatgc aacaactcat
  241 atagggttct agatggaaga aattgcacat taatagatgc aatgctagga gaccctcact
  301 gtgatgtctt tcagtatgag aattgggacc tcttcataga aagaagcagc gctttcagca
  361 attgctaccc atatgacatc cctgactatg catcgctccg gtccattgta gcatcctcag
  421 gaacattaga attcacagca gagggattca catggacagg tgtcactcaa aacggaggaa
  481 gtggagcctg caaaagagga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacaa tgagcagaca aaattgtata
  661 tccaagaatc aggacgagta acagtctcaa caaaaagaag tcaacaaaca ataatcccta
  721 acatcggatc tagaccgtgg gtcaggggtc aatcaggcag gataagcata tactggacca
  781 ttgtaaaacc tggagatatc ctaatgataa acagtaatgg caacttagtt gcaccgcggg
  841 gatattttaa attgagaaca gggaaaagct ctgtaatgag atcagatgca cccatagaca
  901 cttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaactcgga aggaacagga caagctgcag atctaaagag cactcaagca gccatcgacc
1201 agatcaatgg aaaattaaac agagtgattg aaaggaccaa tgagaaattc catcaaatag
1261 agaaggaatt ctcagaagta gaagggagaa tcaaggactt ggagaagtat gtagaagaca
1321 ccaaaataga cctatggtcc tacaatgcag aattgctggt ggctctagaa aatcaacata
1381 caattgactt aacagatgca gaaatgaata aattattcga gaagactaga cgccagttaa
1441 gagaaaacgc ggaagacatg ggaggtggat gtttcaagat ctaccacaaa tgtgataatg
1501 catgcattgg atcaataaga aatgggacat atgaccatta catatacaaa gatgaagcat
1561 taaacaaccg atttcaaatc aaaggtgttg agttgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A virus (A/equine/Kentucky/1/94(H3N8)) hemagglutinin precursor (HA) gene, complete cds.

```
ACCESSION L39914; SEQ ID NO: 19
      1 agcaaaagca ggggatattt ctgtcaatca tgaagacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagagttct agatggaaga aattgcacat taatagatgc aatgctagga gacccccact
  301 gtgatgtctt tcagtatgag aattgggacc tcttcataga aagaagcagc gctttcagca
  361 attgctaccc atatgacatc cctgactatg catcgctccg gtccattgta gcatcctcag
  421 gaacattgga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacca acagcaaaca gaattgtaca
  661 tccaagaatc aggacgagta acagtctcaa caaaaagaag tcaacaaacg ataatcccta
  721 atatcggatc tagaccgtgg gtcaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaactcgga aggaacagga caagctgcag atctaaagag cactcaagca gccatcgacc
1201 agattaatgg aaaattaaac agagtgattg aaaggaccaa tgagaaattc catcaaatag
1261 agaaggaatt ctcagaagta gaagggagaa tccaggactt ggagaagtat gtagaagaca
1321 ccaaaataga cctatggtcc tacaatgcag aattgctggt ggctctagaa aatcaacata
1381 caattgactt aacagatgca gaaatgaata aattattcga gaagactaga cgccagttaa
1441 gagaaaacgc ggaagacatg ggaggtggat gtttcaagat ttaccacaaa tgtgataatg
1501 catgcattgg atcaataaga aatgggacat atgaccatta catatacaga gatgaagcat
1561 taaacaaccg atttcaaatc aaaggtgttg agttgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A virus (A/equine/Kentucky/1/81(H3N8)) nucleoprotein (NP) gene, complete cds.

```
ACCESSION AY291288; SEQ ID NO: 20
      1 agcaaaagca gggtagataa tcactcactg agtgacatca aagtcatggc gtctcaaggc
    61 accaaacgat cttatgagca gatggaaact ggtggggaac gccagaatgc aactgaaatc
  121 agagcatctg ttggaaggat ggtgggagga atcggccggt tctatgttca aatgtgtact
  181 gagcttaaac tcaacgacca tgaagggcgg ctgattcaga acagcataac aatagaaagg
  241 atggtacttt cggcattcga cgaaagaaga aacaagtacc tcgaggagca tcccagtgct
  301 gggaaagacc ccaagaaaac gggaggcccg atatacagaa ggagagatgg gaaatggatg
  361 agagaactca tcctccatga taaagaagaa atcatgagga tctggcgtca ggccaacaat
  421 ggtgaagacg ctactgctgg tcttactcat atgatgatct ggcactccaa tctcaatgac
  481 accacctacc aaagaacaag ggctcttgtt cgggctggga tggatcccag aatgtgctct
  541 ctgatgcaag gatcaactct cccacggaga tctggagctg ccggtgctgc agtgaagggt
  601 gttggaacaa tggtaatgga actcatcagg atgatcaaac gcgggataaa tgatcgaaac
  661 ttctggagag gtgaaaatgg tcgaagaacc agaattgctt atgaaagaat gtgcaacatc
  721 ctcaagggga aattccaaac agcagcacaa cgagcaatga tggaccaagt gagggagggc
  781 cgcaatcctg gaaatgctga gattgaggat ctcattttct tggcacgatc agcactcatt
  841 ctgagaggat cagtagccca taaatcatgc ctacctgcct gtgtttatgg ccttgcagta
  901 gccagtgggt atgactttga gaaagaggga tactctctgg ttggaattga tcctttcaaa
  961 ctactccaga acagccaaat tttcagtcta atcagaccga aagaaaatcc agcacacaag
1021 agccagctgg tgtggatggc atgccattct gcagcatttg aggacctgag agtttcgaat
1081 ttcattagag gaaccaaagt aatcccaaga ggacagttag caaccagggg agtgcaaatt
1141 gcttcaaatg aaaacatgga gacaatagat tctagcacac tcgaactgag gagcagatat
1201 tgggcaataa ggaccaggag tggggggaac accagtcaac agagagcatc tgcaggacag
1261 ataagtgtgc aacccacttt ctcagtgcag agaaatcttc cctttgaaag agcaaccatt
1321 atggctgcat tcactggaaa cactgagggg aggacttccg acatgagaac ggaaatcata
1381 aggatgatgg aaaatgccag atcagaagat gtgtctttcc aggggcgggg agtcttcgag
1441 ctctcggacg aaaaggcaac gaacccgatc gtgccttcct ttgacatgag caatgaaggg
1501 tcttatttct tcggagacaa tgctgaggag tttgacagtt aaagaaaaat acccttgttt
1561 ctact
```

DEFINITION Influenza A virus (A/Equine/Kentucky/1/92(H3N8)) gene for hemagglutinin precursor, partial cds.

```
ACCESSION D30683; SEQ ID NO: 21
      1 gtcaatcatg aagacaacca ttattttgat actactgacc cattgggtct acagtcaaaa
    61 cccaaccagt ggcaacaaca cagccacatt atgtctggga caccatgcag tagcaaatgg
  121 aacattggta aaaacaataa ctgatgacca aattgaggtg acaaatgcta ctgaattagt
  181 tcagagcatt tcaataggga aaatatgcaa caactcatat agggttctag atggaagaaa
  241 ttgcacatta atagatgcaa tgctaggaga cccccactgt gatgtctttc agtatgagaa
  301 ttgggacctc ttcatagaaa gaagcagcgc tttcagcaat tgctacccat atgacatccc
  361 tgactatgca tcgctccggt ccattgtagc atcctcagga acattagaat tcacagcaga
  421 gggattcaca tggacaggtg tcactcaaaa cggaggaagt ggagcctgca aaaggggatc
  481 agccgatagt ttctttagcc gactgaattg gctaacaaaa tctggaaact cttaccccac
  541 attgaatgtg acaatgccta acaataaaaa tttcgacaaa ctatacatct gggggattca
  601 tcacccgagc tcaaacaatg agcagacaaa attgtatatc caagaaacag gacgagtaac
  661 agtctcaaca aaaagaagtc aacaaacaat aatccctaac atcggatcta gaccgtgggt
  721 caggggtcaa tcaggcagga taagcatata ctggaccatt gtaaaacctg gagatatcct
  781 aatgataaac agcaatggca acttagttgc accgcgggga tattttaaat tgagaacagg
  841 gagaagctct gtaatgagat cagatgcacc catagacatt tgtgtgtctg aatgtattac
  901 accaaatgga agcatcccca acgacaaacc atttcaaaat gtgaacaaag ttacatatgg
  961 aaaatgcccc aaatatatca ggcaaaacac tttaaagctg gccactggga tgaggaatgt
1021 accagaaaag caaatcagag gaatctttgg agcaatagcg ggattcatag aaaacggctg
1081 ggaaggaatg gttgat
```

DEFINITION Influenza A virus (A/equine/Kentucky/1/97(H3N8)) hemagglutinin precursor (HA1) mRNA, partial cds.

```
ACCESSION AF197249; SEQ ID NO: 22
      1 agcaaaagca ggggatattt ctgtcaatca tgaagacaac cattattttg atactactga
    61 cccattgggc ctacagtcaa aacccaatca gtggcaacaa cacagccaca ttgtgtctgg
  121 gacaccatgc agtagcaaat ggaacattgg taaaaacaat aactgatgat caaattgagg
  181 tgacaaatgc tactgaatta gttcagagca tttcaatggg gaaaatatgc aacaactcat
  241 atagagttct agatggaaga aattgcacat taatagatgc aatgctagga gacccccact
  301 gtgatgtctt tcagtatgag aattgggacc tctttataga aagaagcagc gctttcagca
  361 attgctaccc atatgacatc cctgactatg catcgctccg gtccattgta gcatcctcag
  421 gaacattgga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacca agagcagaca aaattgtaca
  661 tccaagaatc aggacgagta acagtctcaa caaaaagaag tcaacaaaca ataatcccta
  721 acatcggatc tagaccgtgg gtcaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatctc caacgacaaa ccattccaaa
  961 atgtgaacaa agttacatat ggaaaatgcc ccaagtatat caggcaaaac actttaaagc
1021 tggccactgg gatgaggaat gaaccagaaa agcaaatcag a
```

DEFINITION Influenza A virus (A/equine/Kentucky/1/96(H3N8)) hemagglutinin precursor (HA1) mRNA, partial cds.

```
ACCESSION AF197248; SEQ ID NO: 23
      1 agcaaaagca ggggatattt ctgtcaatca tgaagacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagagttct agatggaaga aattgcacat taatagatgc aatgctagga gacccccact
  301 gtgatgtctt ccagtatgag aattgggacc tcttcataga aagaagcagc gctttcagca
  361 attgctaccc atatgacatc cctgactatg catcgctccg gtccattgta gcatcctcag
  421 gaacattaga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacca aaagcagaca gaattgtaca
  661 tccaagaatc aggacgagta acagtctcaa caaaaagaag tcaacaaacg ataatcccta
  721 atatcggatc tagaccgtgg gttaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat gtaccagaaa agcaaatcag a
```

DEFINITION Influenza A virus (A/equine/Kentucky/9/95(H3N8)) hemagglutinin precursor (HA1) mRNA, partial cds.

```
ACCESSION AF197247; SEQ ID NO: 24
      1 agcaaaagca ggggatattt ctgtcaatca tgaagacaac cattattttg atactactga
    61 cccattgggt ctacagtcaa aacccaacca gtggaaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagagttct agatggaaga aattgcacat taatagatgc aatgctagga gacccccact
  301 gtgatgtctt ccagtatgag aattgggacc tcttcataga aagaagcagc gctttcagca
  361 attgctaccc atatgacatc cctgactatg catcgctccg gtccattgta gcatcctcag
  421 gaacattgga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacca aaagcagaca gaattgtaca
  661 tccaagaatc aggacgagta acagtctcaa caaaaagaag tcaacaaacg ataatcccta
  721 atatcggatc tagaccgtgg gtcaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat gtaccagaaa agcaaatcag a
```

DEFINITION Influenza A virus (A/equine/Kentucky/1/98(H3N8)) hemagglutinin precursor (HA1) mRNA, partial cds.

```
ACCESSION AF197241; SEQ ID NO: 25
      1 agcaaaagca ggggatattt ctgtcaatca tgaagacaac cattattttg atactactga
    61 cccattgggt ctacagtcaa aacccaacca gtggaaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 ataaagttct agatggaaga aattgcacat taatagatgc aatgctagga gacccccact
  301 gtgatgtctt ccagtatgag aattgggacc tcttcataga aagaagcagc gctttcagca
  361 attgctaccc atatgacatc cctgactatg catcgctccg gtccattgta gcatcctcag
  421 gaacattaga attcacagca gagggattca catggacagg tgtcactcaa aacggaagaa
  481 gtggagcctg caaaagggga tcagccgata gtttctttag ccgactgaat tggctaacaa
  541 aatctggaaa ctcttacccc acattgaatg tgacaatgcc taacaataaa aatttcgaca
  601 aactatacat ctgggggatt catcacccga gctcaaacca acagcagaca gaattgtaca
  661 tccaagaatc aggacgagta acagtctcaa caaaaagaag tcaacaaacg atagtcccta
  721 atatcggatc tagaccgtgg gttaggggtc aatcaggcag gataagcata tactggacca
  781 ttgtaaaacc tggagatatc ctaatgataa acagtaatgg caacttagtt gcaccgcggg
  841 gatattttaa attgaaaaca gggaaaagct ctgtaatgag atcagatgca cccatagaca
  901 tttgtgtgtc tgaatgtatt acaccaaatg gaagcatccc caacgacaaa ccatttcaaa
  961 atgtgaacaa agttacatat ggaaaatgcc ccaagtatat caggcaaaac actttaaagc
1021 tggccactgg gatgaggaat ataccagaaa agcaaatcag a
```

DEFINITION Influenza A virus (A/eq/Kentucky/81(H3N8)) hemagglutinin mRNA, complete cds.

```
ACCESSION U58195; SEQ ID NO: 26
      1 agcaaaagca ggggatactt tctgtcaatc atgaagacaa ccattatttt gatactactg
    61 acccattggg tctacagtca aaacccaacc agtggcaaca acacagccac actatgtctg
  121 ggacaccatg cagtagcaaa tggaacattg gtaaaaacaa taactgatga ccaaattgag
  181 gtgacaaatg ctactgaatt agttcagagc acttcaatag ggaaaatatg caacaaccca
  241 tatagggttc tagatggaag aaactgcaca ttaatagatg caatgctagg agatccccac
  301 tgtgatgttt ttcagtatga gaattgggac ctcttcatag aaagaagcag cgctttcagc
  361 aattgctacc catatgacat ccctgactat gcatcgctcc ggtctattgt ggcatcttca
  421 ggaacattag aattcacagc agagggattc acatggacag gtgtcactca aaacggagga
  481 agtggagcct gcagaagggg gtcagccgat agtttcttta gccgactgaa ttggctaaca
  541 aaatctggaa attcttaccc cacattgaat gtaacaatgc ctaacaataa caatttcgat
  601 aaactataca tctgggggat ccatcacccg agcacaaaca atgagcagac aaaattgtat
  661 atccaagaat cagggcgagt aacagtctca acaaaaagaa gtcaacaaac aataatcccc
  721 aacatcggat ctagaccgtg ggtcaggggt caatcaggca ggataagcat atattggacc
  781 attgtgaaac ctggagatat cctaatgata aacagtaatg gcaacttagt tgcaccgcgg
  841 ggatatttta aaatgcgaac agggaaaagc tctgtaatga gatcagatgc acccatagac
  901 acttgtgtgt ccgagtgtat tacaccaaat ggaagcatcc ccaacgacaa accatttcaa
  961 aatgtgaaca aagttacata tggaaaatgc cccaagtata tcaagcagaa tactttgaag
1021 ctggccactg ggatgaggaa tgtaccagaa aagcaaatca gaggaatctt tggagcaata
1081 gcgggattca tagaaaacgg ctgggaagga atggttgatg ggtggtatgg attccgatat
1141 cagaattcgg aaggaacagg acaagctgca gatctaaaga gcactcaagc agccatcgac
1201 cagatcaatg gaaaattgaa cagagtgatt gaaaggacca atgagaaatt ccatcaaata
1261 gagaaggaat tctcagaagt agaagggaga atccaggact tggagaagta tgtagaagac
1321 accaaaatag acctatggtc ctacaatgca gagttactgg tggctctaga aaatcaacat
1381 acgattgact taacagatgc agaaatgaat aaattattcg agaagactag gcgccagtta
1441 agagaaaacg cggaagacat ggggggtgga tgtttcaaga tttatcacaa atgtgataat
1501 gcatgcattg gatcaataag aaatgggaca tatgaccatt acatatacag agatgaagca
1561 ttaaacaacc gatttcaaat taaaggtgtt gaattgaaat caggctacaa agattggata
1621 ctgtggattt cattcgccat atcatgcttc ttaatttgcg ttgttctatt gggtttcatc
1681 atgtgggctt gccaaaaagg caacatcaga tgcaacattt gcatttgagt aaactgataa
1741 ttaaaaacac ccttgtttct act
```

DEFINITION Influenza A virus (A/equine/Kentucky/2/86 (H3N8)) membrane protein M1 and membrane protein M2 genes, complete cds.

```
ACCESSION M63540; SEQ ID NO: 27
      1 agcaaaagca ggtagatatt taaagatgag tcttctaacc gaggtcgaaa cgtacgttct
    61 ctctattgta ccatcaggcc ccctcaaagc cgagatcgcg cagagacttg aagatgtctt
  121 tgcagggaag aacaccgatc ttgaggcact catggaatgg ctaaagacaa gaccaatcct
  181 gtcacctctg actaaaggga ttttaggatt tgtgttcacg ctcaccgtgc ccagtgagcg
  241 aggactgcaa cgtagacgct ttgtccaaaa tgcccttagt ggaaacggag atccaaataa
  301 catggacaga gcagtaaaac tgtacaagaa gcttaaaaga gaaataacat tccatggggc
  361 aaaagaggtg gcactcagct attccactgg tgcactagcc agctgcatgg gactcatata
  421 caacagaatg gggactgtga caaccgaagt ggcatttggc ctggtatgcg ccacatgtga
  481 acagattgct gattcccagc atcgatctca caggcagatg gtgacaacaa ccaacccact
  541 aatcagacat gaaaacagaa tggtactagc cagtaccaca gctaaaacca tggagcaggt
  601 ggcagggtcg agtgagcagg cagcagaggc catggaggtt gctagtaagg ccaggcagat
  661 ggtgcaggca atgaggacca ttgggaccca ccctagctcc agtgccggtt tgaaagatga
  721 tcttcttgaa aatttgcagg cctaccagaa acggatggga gtgcaaatgc agcggttcaa
  781 gtgatcctct cgttattgca gcaagtatca ttgggatctt gcacttgata ttgtggattc
  841 ttgatcgcct tttcttcaaa ttcatttatc gtctccttaa atacggtttg aaaagagggc
  901 cttctacgga aggagtacct gagtctatga gggaagaata tcggcaggaa cagcagaatg
  961 ctgtggatgt tgacgatggt cattttgtca acatagagct ggagtaaaaa actaccttgt
1021 ttctact
```

DEFINITION Influenza A virus isolate A/equine/Kentucky/76 nonstructural protein, complete cds.

```
ACCESSION M80971; SEQ ID NO: 28
    1 agcaaaagca gggtgacaaa aacataatgg attccaacac tgtgtcaagc tttcaggtag
  61 actgttttct ttggcatgtc cgcaaacgat ttgcagacca agaactgggt gatgccccat
121 tccttgaccg gcttcgccga gaccagaagt ccctaaaagg aagaggcagc actcttggtc
181 tggacatcga aacagccact cgtgcaggaa agcagatagt ggagcggatt ctggaagagg
241 agtcagatga ggcacttaaa atgaccattg cctctgttcc tgcttcacgc tacttaactg
301 acatgactct tgatgagatg tcaagagact ggttcatgct catgcccaag cagaaagtaa
361 caggctccct atgtataagg atggaccagg caatcatgga taagaacatc atactaaaag
421 caaactttag tgtgattttc gaaaggctgg agacactaat actacttaga gctttcaccg
481 aagaaggagc agtcgttggc gaaatttcac cattgccttc tcttccagga catactaatg
541 aggatgtcaa aaatgcaatt ggggtcctca tcggaggact taaatggaat gataacacag
601 ttagaatctc tgaaactcta cagagattcg cttggagaag cagtcatgag aatgggagac
661 cttcattccc tccaaagcag aaacgaaaaa tggcgagaac aattgagtca gaagtttgaa
721 gaaataaggt ggttgattga agaagtgcga catagattga aaaatacaga aaatagtttt
781 gaacaaataa catttatgca agccttacaa ctattgcttg aagtagaaca agagataaga
841 actttctcgt ttcagcttat ttaatgataa aaaacaccct tgtttctact
```

DEFINITION Influenza A virus (A/eq/Kentucky/92(H3N8)) matrix proteins M1 and M2 (M) gene, complete cds.

```
ACCESSION AF001683; SEQ ID NO: 29
    1 atgagtcttc tgaccgaggt cgaaacgtac gttctctcta tcgtaccatc aggccccctc
  61 aaagccgaga tcgcgcagag acttgaagat gtctttgcag ggaagaacac cgatcttgag
121 gcactcatgg aatggctaaa gacaagacca atcctgtcac ctctgactaa agggatttta
181 ggattcgtat tcacgctcac cgtgcccagt gagcgaggac tgcagcgtag acgctttgtc
241 caaaatgccc ttagtggaaa cggagatcca aacaacatgg acagagcagt aaaactgtac
301 aggaaactta aaagagaaat aacattccat ggggcaaaag aggtggcact cagctattcc
361 actggtgcac tagccagctg catgggactc atatacaaca gaatgggaac tgtgacaacc
421 gaagtggcat ttggcctagt atgcgccaca tgtgaacaga ttgctgattc ccagcatcga
481 tctcacaggc agatggtgac aacaaccaac ccattaatca gacatgaaaa cagaatggta
541 ttagccagta ccacggctaa agccatggag cagatggcag ggtcgagtga gcaggcagca
601 gaggccatgg aggttgctag taaggctagg cagatggtac aggcaatgag gaccattggg
661 acccacccta gctccagtgc cggtttgaaa aatgatctcc ttgaaaattt gcaggcctac
721 cagaaacgga tgggagtgca aatgcagcga ttcaagtgat cctctcgtta ttgcagcaag
781 tatcattggg atcttgcact tgatattgtg gattcttgat cgccttttct tcaaattcat
841 ttatcgtcgc cttaaatacg ggttgaaaag agggccttct acggaaggag tacctgagtc
901 tatgagggaa gaatatcggc aggaacagca gaatgctgtg gatgttgacg atggtcattt
961 tgtcaacata gagctggagt aa
```

DEFINITION Influenza A virus (A/eq/Kentucky/81 (H3N8)) matrix proteins M1 and M2 (M) gene, complete cds.

```
ACCESSION AF001676; SEQ ID NO: 30
    1 atgagtcttc taaccgaggt cgaaacgtac gttctctcta tcgtaccatc aggccccctc
  61 aaagccgaga tcgcgcagag acttgaagat gtctttgcag ggaagaacac cgatcttgag
121 gcactcatgg aatggctaaa gacaagacca atcctgtcac ctctgactaa agggatttta
181 ggatttgtgt tcacgctcac cgtgcccagt gagcgaggac tgcagcgtag acgctttgtc
241 caaaatgccc ttagtggaaa cggagatcca aacaacatgg acagagcagt aaaactgtac
301 aggaagctta aaagagaaat aacattccat ggggcaaaag aggtggcact cagctattcc
361 actggtgcac tagccagctg catgggactc atatacaaca gaatggggac tgtgacaacc
421 gaagtggcat ttggcctggt atgcgccaca tgtgaacaga ttgctgattc ccagcatcga
481 tctcacaggc agatggtgac aacaaccaac ccactaatca gacatgaaaa cagaatggta
541 ctagccagta ccacagctaa agccatggaa cagatggcag ggtcgagtga gcaggcagca
601 gaggccatgg aggttgctag taaggccagg cagatggtac aggcaatgag gaccattggg
661 acccacccta gctccagtgc cggtttgaaa gatgatcttc ttgaaaattt gcaggcctac
721 cagaaacgga tgggagtgca aatgcagcga ttcaagtgac cctctcgtta ttgcagcaag
781 tatcattggg atcttgcact tgatattgtg gattcttgat cgccttttct tcaaattcat
841 ttatcgtcgc cttaaatacg gtttgaaaag agggccttct acggaaggag tacctgagtc
901 tatgagggaa gaatatcggc aggaacagca gaatgctgtg gatgttgacg atggtcattt
961 tgtcaacata gagctggagt aa
```

DEFINITION Influenza A virus (A/eq/Kentucky/92(H3N8)) nonstructural proteins NS1 and NS2 (NS) gene, complete cds.

```
ACCESSION AF001671; SEQ ID NO: 31
    1 atggattcca acactgtgtc aagctttcag gtagactgtt ttctttggca tgtccgcaaa
  61 cgattcgcag accaagaact gggtgatgcc ccattccttg accggcttcg ccgagaccag
121 aagtccctaa aaggaagagg tagcactctt ggtctggaca tcgaaacagc cactcgtgca
181 ggaaagcaga tagtggagca gattctggaa gaggaatcag atgaggcact taaaatgacc
241 attgcctctg ttcctgcttc acgctactta actgacatga ctcttgatga gatgtcaaga
301 gactggttca tgctcatgcc caagcagaaa gtaacaggct ccctatgtat aagaatggac
361 caggcaatca tggataagaa catcatactt aaagcaaact ttagtgtgat tttcgaaagg
421 ctggaaacac taatactact tagagccttc accgaagaag gagcagtcgt tggcgaaatt
481 tcaccattgc cttctcttcc aggacatact aatgaggatg tcaaaaatgc aattggggtc
541 ctcatcggag gacttaaatg gaatgataat acggttagaa tctctgaaac tctacagaga
601 ttcgcttgga gaagcagtca tgagaatggg agaccttcat tccctccaaa gcagaaacga
661 aaaatggaga gaacaattga gccagaagtt tgaagaaata agatggttga ttgaagaagt
721 gcgacataga ttgaaaaata cagaaaatag ttttgaacaa ataacattta tgcaagcctt
781 acaactattg cttgaagtag aacaagagat aagaactttc tcgtttcagc ttatttaa
```

DEFINITION Influenza A virus (A/eq/Kentucky/1/88(H3N8)) nonstructural proteins NS1 and NS2 (NS) gene, complete cds.

```
ACCESSION AF001664; ; SEQ ID NO: 32
    1 atggattcca acactgtgtc aagctttcag gtagactgtt ttctttggca tgtccgcaaa
  61 cgatttgcag accaagaact gggtgatgcc ccattccttg accggcttcg ccgagaccag
121 aagtccctaa aaggaagagg cagcactctt ggtctggaca tcgaaacagc cactcgtgca
181 ggaaagcaga tagtggagca gattctggaa gaggaatcag atgaggcact taaaatgacc
241 attgcctctg ttcctgcttc acgctactta actgacatga ctcttgatga gatgtcaaga
301 gactggttca tgctcatgcc caagcagaaa gtaacaggct ccctatgtat aaggatggac
361 caggcaatca tggataagaa catcatacta aaagcaaact ttagtgtgat tttcgaaagg
421 ctggagacac taatactact tagagccttc accgaagaag gagcagtcgt tggcgaaatt
481 tcaccattgc cttctcttcc aggacatact aatgaggatg tcaaaaatgc aattggggtc
541 ctcatcggag gacttaaatg gaatgataat acagttagag tctctgaaac tctacagaga
601 ttcgcttgga gaagcagtca tgagaatggg agaccttcat tccctccaaa gcagaaacga
661 aaaatggcga gaacaattga gccagaagtt tgaagaaata agatggttga ttgaagaagt
721 gcgacataga ttgaaaaata cagaaaatag ttttgaacaa ataacattta tgcaagcctt
781 acaactattg cttgaagtag aacaagagat aagaactttc tcgtttcagc ttatttaa
```

DEFINITION Influenza A/equine/Kentucky/2/86 (H3N8) nucleoprotein (seg 5) mRNA, complete cds.

```
ACCESSION M30751; SEQ ID NO: 33
      1 agcaaaagca gggtagataa tcactcactg agtgacatca aagtcatggc gtctcaaggc
    61 accaaacgat cttatgagca gatggaaact ggtggggaac gccagaatgc aactgaaatc
  121 agagcatctg tcggaaggat ggtgggagga atcggccggt tctatgttca gatgtgtact
  181 gagcttaaac tcaacgacca tgaagggcgg ctgattcaga acagcataac aatagaaagg
  241 atggtacttt cggcattcga cgaaagaaga aacaagtacc tcgaggagca tcccagtgct
  301 gggaaagacc ccaagaaaac gggaggcccg atatacagaa ggaaagatgg gaaatggatg
  361 agagaactca tcctccatga taaagaagaa atcatgagga tctggcgtca ggccaacaat
  421 ggtgaagacg ctactgctgg tcttactcat atgatgatct ggcactccaa tctcaatgac
  481 accacatacc aaagaacaag ggctcttgtt cgggctggga tggatcccag aatgtgctct
  541 ctgatgcaag gatcaaccct cccacggaga tctggagctg ccggtgctgc agtaaaaggt
  601 gttggaacaa tggtaatgga actcatcagg atgatcaaac gcgggataaa tgatcgaaat
  661 ttctggagag gtgaaaatgg tcgaagaacc agaattgctt atgaaagaat gtgcaatatc
  721 ctcaaaggga aattccaaac agcagcacaa cgggcaatga tggaccaagt gagggagggc
  781 cgcaatcctg gaaatgctga gattgaggat ctcattttct tggcacgatc agcactcatt
  841 ttgagaggat cagtagccca taaatcatgc ctacctgcct gtgtttatgg ccttgcagta
  901 gccagtgggt atgactttga gaaggaagga tactctctgg ttggaattga tcctttcaaa
  961 ctactccaga acagccaaat tttcagtcta atcagaccga aagaaaatcc agcacacaag
1021 agccagttgg tgtggatggc atgccattct gcagcatttg aggacctgag agttttgaat
1081 ttcattagag gaaccaaagt aatcccaaga ggacagttag caaccagagg agtgcaaatt
1141 gcttcaaatg aaaacatgga gacaatagat tctagcacac tcgaactgag gagcagatat
1201 tgggcaataa ggaccaggag tggagggaac accagtcaac agagagcatc tgcaggacag
1261 ataagtgtgc aacccacttt ctcagtgcag agaaatcttc cctttgaaag agcaaccatt
1321 atggctgcat tcactgggaa cactgagcgg aggacttccg acatgagaac ggaaatcata
1381 aggatgatgg aaaatgccag atcagaagat gtgtctttcc aggggcgggg agtcttcgag
1441 ctctcggacg aaaaggcaac gaacccgatc gtgccttcct ttgacatgag caatgaaggg
1501 tcttatttct tcggagacaa tgctgaggag tttgacagtt aaagaaaaat acccttgttt
1561 ctact
```

DEFINITION Influenza A/Equine/Kentucky/2/86 (H3N8), PB2 polymerase, complete cds.

```
ACCESSION M73526 M36049; SEQ ID NO: 34
      1 agcaaaagca ggtcaaatat attcaatatg gagagaataa aagaactgag agatctaatg
    61 tcacagtccc gcacccgcga gatactaaca aaaactactg tggaccatat ggccataatc
  121 aagaaataca catcaggaag acaagagaag aaccccgcac ttaggatgaa gtggatgatg
  181 gcaatgaaat acccaattac agcagataag aggataatgg aaatgattcc tgagagaaat
  241 gaacaggggc aaaccctttg gagcaaaacg aacgatgctg gctcagaccg cgtaatggta
  301 tcacctctgg cagtgacatg gtggaatagg aatggaccaa caacgagcac aattcattat
  361 ccaaaagtct acaaaactta ttttgaaaaa gttgaaaggt taaaacacgg aacctttggc
  421 cccgttcatt ttaggaatca agtcaagata agacggagag ttgacgtaaa ccctggtcac
  481 gcggacctca gtgccaaaga agcacaagat gtgatcatgg aagttgtttt cccaaatgaa
  541 gtgggagcca gaattctaac atcggaatca caactaacaa taaccaaaga gaaaaaagaa
  601 gaacttcagg actgcaaaat tgcccccttg atggtagcat acatgctaga aagagagttg
  661 gtccgaaaaa caaggttcct cccagtggct ggcggaacaa gcagtgtata cattgaggtg
  721 ttgcatctga ctcagggaac gtgctgggaa caaatgtaca ccccaggagg agaagttaga
  781 aacgatgaca ttgatcaaag tttaattatt gctgcccgga acatagtgaa aagagcgaca
  841 gtatcagcag atccactagc atccctgctg gagatgtgcc acagtacaca gattggtgga
  901 ataaggatgg tagacatcct taagcagaat ccaacagagg aacaagctgt ggatatatgc
  961 aaagcagcaa tggggttaag aattagctca tcattcagct ttggtggatt cacctttaag
1021 agaacaagtg gatcatcagt caagagagaa gaagaaatgc ttacgggcaa ccttcaaaca
1081 ttgaaaataa gagtgcatga gggctatgaa gaattcacaa tggtcggaag aagagcaaca
1141 gccattctca gaaagacaac cagaagattg attcaattga tagtaagtgg gagagatgaa
1201 cagtcaattg ctgaagcaat aattgtagcc atggtgtttt cgcaagaaga ttgcatgata
1261 aaagcagttc gaggcgattt gaacttcgtt aatagagcaa atcagcgctt gaaccccatg
1321 catcaactct tgaggcattt ccaaaaggat gcaaaagtgc ttttccagaa ttgggggatt
1381 gaacccatcg acaatgtgat gggaatgatc ggaatattgc ccgacatgac cccaagcacc
1441 gagatgtcat tgagaggagt gagagtcagc aaaatgggag tggatgagta ctccagcact
1501 gagagagtgg tggtgagcat tgaccgtttt ttaagagttc gggatcaaag gggaaacata
1561 ctactgtccc ctgaagaggt cagtgaaaca caaggaacgg aaaagctgac aataatttat
1621 tcatcatcaa tgatgtggga gattaatggt cccgagtcag tgttggtcaa tacttatcaa
1681 tggatcatca gaaactggga aattgtgaaa attcaatggt cacaggatcc cacaatgtta
1741 tacaataaga tagaatttga gccattccag tccctggtcc ctagggccac cagaagccaa
1801 tacagcggtt tcgtaaggac cctgtttcag caaatgcgag atgtacttgg aacatttgac
1861 actgctcaaa taataaaact cctccctttt gccgctgctc ctccggaaca gagtagaatg
1921 cagttctctt ctttgactgt taatgtaaga ggatcgggaa tgaggatact tgtaagaggc
1981 aattccccag tgttcaacta caacaaagcc actaagaggc tcacagtcct cggaaaggat
2041 gcaggtgcgc ttactgaaga cccagatgaa ggtacggctg gagtagaatc tgctgttctg
2101 agagggtttc tcatcttagg taaagaaaac aagagatatg gcccagcact aagcatcaat
2161 gaactgagca aacttacaaa aggggagaaa gctaatgtgc taattgggca aggggacgtg
2221 gtgttggtaa tgaaacggaa acgtgactct agcatactta ctgacagtca gacagcgacc
2281 aaaaggattc ggatggccat caattagtgt tgaattgttt aaaaacgacc ttgtttctac
2341 t
```

DEFINITION Influenza A/equine/Kentucky/1/87 (H3N8) hemagglutinin (HA) RNA (seg. 4), complete cds.

```
ACCESSION M24728 J04336; SEQ ID NO: 35
      1 agcaaaagca ggggatattt ctgtcaatca tgaagacaac cattgttttg atactactga
    61 cccattgggt ctacagtcaa aacccaacca gtggcaacaa cacagccaca ttatgtctgg
  121 gacaccatgc agtagcaaat ggaacactgg taaaaacaat aactgatgac cagattgagg
  181 tgacaaatgc tactgaatta gttcagagca tttcaatagg gaaaatatgc aacaactcat
  241 atagggttct agatggaaga aattgcacat taatagatgc aatgctagga gacccccact
  301 gtgatgtttt tcngtatgag aattgggacc tcttcataga aagaagcagc gctttcagca
  361 attgctaccc atatgacatc cctgactatg catcgctccg gtctattgtg gcatcctcag
  421 gaacattaga attcacagca gagggattca catggacagg tgtcactcaa aacggaagaa
  481 gtggagcctg caaaagggga tcagccgata gtttctttag ccgactgaat tggctaacaa
  541 aatctggaaa ttcttacccc acattgaatg tgacaatgcc taacaataac aatttcgata
  601 aactatacat ctgggggatt catcacccga gctcaaacaa tgagcagaca aaattgtata
  661 tccaagaatc aggacgagta acagtctcaa caaaaagaag tcaacaaaca ataatcccca
  721 acatcggatc tagaccgtgg gtcaggggtc aatcaggcag gataagcata tattggacca
  781 ttgtgaaacc tggagatatc ctaatgataa acagtaatgg caacttagtt gcaccgcggg
  841 gatatttcaa attgagaaca gggaaaagct ctgtaatgag atcagatgca cccatagaca
  901 cttgtgtgtc tgaatgtatt acaccaaatg gaagcatccc caacgacaaa ccattccaaa
  961 atgtgaacaa agttacatat ggaaaatgcc ccaagtatat caggcaaaac actttgaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaattcgga aggaacagga caagctggag atctaaagag cactcaagca gccatcgacc
1201 agatcaatgg aaaattaaac agagtgattg aaaggaccaa tgagaaattc catcaaatag
1261 agaaggaatt ctcagaagta gaagggagaa tccaggactt ggagaagtat gtagaagaca
1321 ccaaaataga cctatggtcc tacaatgcag aattgctggt ggctctagaa aatcaacata
1381 caattgactt aacagatgca gaaatgaata aattattcga gaagactagg cgccagttaa
1441 gagaaaacgc ggaagacatg ggaggtggat gtttcaggat ttaccacaaa tgtgataatg
1501 catgcattgg atcaataaga aatgggacat atgaccatta catatacaga gatgaagcat
1561 taaacaaccg atttcaaatt aaaggtgttg agttgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

DEFINITION Influenza A/equine/Kentucky/2/86 (H3N8) hemagglutinin (HA) RNA (seg. 4), complete cds.

```
ACCESSION M24727 J04336; SEQ ID NO: 36
      1 agcaaaagca ggggatattt ctgtcaatca tgaagacaac cattattttg atactactga
    61 cccattgggt ctacagtcaa aacccaacca gtggcaacaa cacagccaca ttatgtctgg
  121 gacaccatgc agtagcaaat ggaacattgg taaaaacaat aactgatgac caaattgagg
  181 tgacaaatgc tactgaatta gttcagagca tttcaatagg gaaaatatgc aacaactcat
  241 atagggttct agatggaaga aattgcacat taatagatgc aatgctagga gacccccact
  301 gtgatgtttt tcngtatgag aattgggacc tcttcataga aagaagcagc gcttccagca
  361 attgctaccc atatgacatc cctgactatg catcgctccg gtctattgtg gcatcctcag
  421 gaacattaga attcacagca gagggattca catggacagg tgtcactcaa aacggaagaa
  481 gtggagcctg caaaagggga tcagccgata gtttctttag ccgactgaat tggctaacaa
  541 aatctggaaa ttcttacccc acattgaatg tgacaatgcc taacaataac aatttcgata
  601 agctatacat ctgggggatc catcacccga gctcaaacaa tgagcagaca aaattgtata
  661 tccaagaatc aggacgagta acagtctcaa caaaaagaag tcaacaaaca ataatcccca
  721 acatcggatc tagaccgtgg gtcaggggtc aatcaggcag gataagcata tattggacca
  781 ttgtgaaacc tggagatatc ctaataataa acagtaatgg caacttagtt gcaccgcggg
  841 gatatttcaa attgcgaaca gggaaaagct ctgtaatgag atcagatgca cccatagaca
  901 cttgtgtgtc tgaatgtatt acaccaaatg gaagcatccc caacgacaaa ccatttcaaa
  961 atgtgaacaa agttacatat ggaaaatgcc ccaagtatat caggcaaaac actttgaagc
1021 tggccactgg gatgaggaat gtaccagaaa agcaaatcag aggaatcttt ggagcaatag
1081 cgggattcat agaaaacggc tgggaaggaa tggttgatgg gtggtatgga ttccgatatc
1141 aaaactcgga aggaacagga caagctggag atctaaagag cactcaagca gccatcgacc
1201 agatcaatgg aaaattgaac agagtgattg aaaggaccaa tgagaaattc catcaaatag
1261 agaaggaatt ctcagaagta gaagggagaa tccaggactt ggagaagtat gtagaagaca
1321 ccaaaataga cctatggtcc tacaatgcag agttgctggt ggctctagaa aatcaacata
1381 caattgactt aacagatgca gaaatgaata aactattcga gaagactagg cgccagttaa
1441 gagaaaacgc ggaagacatg ggaggtggat gtttcaagat ttatcacaaa tgtgataatg
1501 catgcattgg atcaataaga aatgggacat atgaccatta catatacaga gatgaagcat
1561 taaacaaccg atttcaaatt aaaggtgtag agctgaaatc aggctacaaa gattggatac
1621 tgtggatttc attcgccata tcatgcttct taatttgcgt tgttctattg ggtttcatta
1681 tgtgggcttg ccaaaaaggc aacatcagat gcaacatttg catttgagta aactgatagt
1741 taaaaacacc cttgtttcta ct
```

### EXAMPLE 5

### Newmarket Equine Influenza Nucleotide Sequences

DEFINITION Influenza A virus (A/equine 2/Suffolk/89(H3N8)) NS1 gene.

```
ACCESSION X80060; SEQ ID NO: 37
    1 atggattcca acactgtgtc aagctttcag gtagactgtt ttctttggca tgtccgcaaa
  61 cgatttgcag accaagaact gggtgatgcc ccattccttg accggcttcg ccgagaccag
121 aagtccctaa aaggaagagg tagcactctt ggtctggaca tcgaaacagc cactcgtgca
181 ggaaagcaga tagtggagca gattctggaa gaggaatcag atgaggcatt taaaatgacc
241 attgcctctg ttcctgcttc acgctactta actgacatga ctcttgatga gatgtcaaga
301 gactggttca tgctcatgcc caagcagaaa gtaacaggct ccctatgtat aagaatggac
361 caggcaatca tggataagaa catcatactt aaagcaaact ttagtgtgat tttcgaaagg
421 ctggagacac taatactact cagggccttc accgaagaag gagcagtcgt tggcgaaatt
481 tcaccattgc cttctcttcc aggacatact aatgaggatg tcaaaaatgc aattggggtc
541 ctcatcggag gacttaaatg gaatgataat acggttagag tctctgaaac tctacagaga
601 ttcgcttgga gaagcagtca tgagaatggg agaccttcat tccctccaaa gcagaaacga
661 aaaatggaga gaacaattga gtcagaagtt tga
```

DEFINITION Influenza A virus (A/eq/Newmarket/93/(H3N8)) HA1 gene for HAY subunit of haemagglutinin, genomic RNA.

```
ACCESSION X85089; SEQ ID NO: 38
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agtccagagc
  181 atttcaatag ggaaaatatg caacaactca tatagggttc tagatggaag aaattgcaca
  241 ttaatagatg caatgctagg agacccccat tgtgatgatt ttcagtatga gaattgggac
  301 ctcttcatag aaagaagcag cgctttcagc aattgctacc catatgacat ccctgactat
  361 gcatcgctcc ggtccattgt agcatcctca ggaacattag aattcacagc agaggggttc
  421 acatggacag gtgtcactca aaacggagga agtggagcct gcaaaagggg atcagccgat
  481 agtttcttta gccgactgaa ttggctaaca aaatctggaa attcttaccc catattgaat
  541 gtgacaatgc ctaacaataa aaatttcgat aaactataca tctgggggat tcatcacccg
  601 agctcaaaca aagagcagac aaaattatat atccaagaat caggacgagt aacagtctca
  661 acagaaagaa gtcaacaaac agtaatccct aacatcggat ctaggccgtg ggtcaggggt
  721 caatcaggca ggataagcat atactggacc attgtaaaac ctggagatat tctaatgata
  781 aacagtaatg gcaacttagt tgcaccgcgg ggatatttta aattgagaac agggaaaagc
  841 tctgtaatga gatcagatgc actcatagac acttgtgtgt ctgaatgtat tacaccaaat
  901 ggaagcatcc ccaacgacaa accatttcaa aatgtgaaca aaattacata tggaaaatgc
  961 cccaagtata tcaggcaaaa cactttaaag ctggccactg ggatgaggaa tgtaccagaa
1021 aagcaaatca ga
```

DEFINITION Influenza A virus (A/eq/Newmarket/93/(H3N8)) HA1 gene for HAY subunit of haemagglutinin, genomic RNA.

```
ACCESSION X85088; SEQ ID NO: 39
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agttcagagc
  181 atttcaatag ggaaaatatg caacaactca tatagagttc tagatggaag aaattgcaca
  241 ttaatagatg caatgctagg agacccccac tgtgatgtct ttcagtatga gaattgggac
  301 ctcttcatag aaagaagcag cgctttcagc aattgctacc catatgacat ccctgactat
  361 gcatcgctcc ggtccattgt agcatcctca ggaacattgg aattcacagc agagggattc
  421 acatggacag gtgtcactca aaacggaaga agtggagcct gcaaaagggg atcagccgat
  481 agtttcttta gccgactgaa ttggctaaca aaatctggaa actcttaccc cacattgaat
  541 gtgacaatgc ctaacaataa aaatttcgac aaactataca tctgggggat tcatcacccg
  601 agctcaaacc aacagcagac agaattgtac atccaagaat caggacgagt aacagtctca
  661 acaaaaagaa gtcaacaaac gataatccct aatatcggat ctagaccatg ggtcaggggt
  721 caatcaggca ggataagcat atactggacc attgtaaaac ctggagatat cctaatgata
  781 aacagtaatg gcaacttagt tgcaccgcgg ggatatttta aattgaaaac agggaaaagc
  841 tctgtaatga gatcagatgc acccatagac atttgtgtgt ctgaatgtat tacaccaaat
  901 ggaagcatcc ccaacgacaa accatttcaa aatgtgaaca aagttacata tggaaaatgc
  961 cccaagtata tcaggcaaaa cactttaaag ctggccactg ggatgaggaa tgtaccagaa
1021 aagcaaatca ga
```

DEFINITION Influenza A virus (A/eq/Sussex/89/(H3N8)) HA1 gene for HAY subunit of haemagglutinin, genomic RNA.

```
ACCESSION X85090; SEQ ID NO: 40
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agttcagagc
  181 atttcaatag ggaaaatatg caacaactca tatagggttc tagatggaag aaattgcaca
  241 ttaatagatg caatgctagg agacccccac tgtgatgttt ttcagtatga gaattgggac
  301 ctcttcatag aaagaagcag cgctttcagc aattgctacc catatgacat ccctgactat
  361 gcatcgctcc ggtccattgt agcatcctca ggaacattag aattcacagc agagggattc
  421 acatggacag gtgtcactca aaacggaaga agtggagcct gcaaaagggg atcagccgat
  481 agtttcttta gccgactgaa ttggctaaca aaatctggaa attcttaccc catattgaat
  541 gtgacaatgc ctaacaataa aaatttcgat aaactataca tctgggggat tcatcacccg
  601 agctcaaaca aagagcagac aaaattgtat atccaagaat caggacgagt aacagtctca
  661 acagaaagaa gtcaacaaac agtaatccct aacatcggat ctagaccgtg ggtcaggggt
  721 caatcaggca ggataagcat atactggacc attgtaaaac ctggagatat tctaatgata
  781 aacagtaatg gcaacttagt tgcaccgcgg ggatatttta aattgagaac agggaaaagc
  841 tctgtaatga gatcagatgc actcataggc acttgtgtgt ctgaatgtat tacaccaaat
  901 ggaagcatcc ccaacgacaa accatttcaa aatgtgaaca aagttacata tggaaaatgc
  961 cccaagtata tcaggcaaaa cactttaaag ctggccactg ggatgaggaa tgtaccagaa
1021 aagcaaatca ga
```

DEFINITION Influenza A virus (A/eq/Lamboum/92/(H3N8)) HA1 gene for HAY subunit of haemagglutinin, genomic RNA.

```
ACCESSION X85087; SEQ ID NO: 41
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agttcagagc
  181 atttcaatag ggaaaatatg caacaactca tatagggttc tagatggaag aaattgcaca
  241 ttaatagatg caatgctagg agacccccat tgtgatgatt ttcagtatga gaattgggac
  301 ctcttcatag aaagaagcag tgctttcagc aattgctacc catatggcat ccctgactat
  361 gcatcgctcc ggtccattgt agcatcctca ggaacattag aattcacagc agaggggttc
  421 acatggacag gtgtcactca aaacggaaga agtggagcct gcaaaagggg atcagccgat
  481 agtttcttta gccgactcaa ttggctaaca aaatctggaa attcttaccc catattgaat
  541 gtgacaatgc ctaacaataa aaatttcgat aaactataca tctgggggat tcatcacccg
  601 agctcaaaca aagagcagac aaaattatat atccaagaat caggacgagt aacagtctca
  661 acagaaagaa gtcaacaaac agtaatccct aacatcggat ctaggccgtg ggtcaggggt
  721 caatcaggca ggataagcat atactggacc attgtaaaac ctggagatat tctaatgata
  781 aacaataatg gcaacttagt tgcaccgcgg ggatatttta aattgagaac agggaaaagc
  841 tctgtaatga gatcagatgc actcatagac acttgtgtgt ctgaatgtat tacaccaaat
  901 ggaagcatcc ccaacgacaa accatttcaa aatgtgaaca aagttacata tggaaaatgc
  961 cccaagtata tcaggcaaaa cactttaaag ctggccactg ggatgaggaa tataccagaa
1021 aagcaaatca ga
```

DEFINITION Influenza A virus (A/eq/Ella/89/(H3N8)) HA1 gene for HAY subunit of haemagglutinin, genomic RNA.

```
ACCESSION X85086; SEQ ID NO: 42
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agttcagagc
  181 atttcaatag ggaaaatatg caacaactca tatagggttc tagatggaag aaattgcaca
  241 ttaatagatg caatgctagg agacccccac tgtgatgttt ttcagtatga gaattgggac
  301 ctcttcatag aaagaagcag cgctttcagc aattgctacc catatgacat ctctgactat
  361 gcatcgctcc ggtccattgt agcatcctca ggaacattag aattcacagc agagggattc
  421 acatggacag gtgtcactca aaacggaaga agtggagcct gcaaaagggg atcagccgat
  481 agtttcttta gccgactgaa ttggctaaca aaatctggaa attcttaccc catattgaat
  541 gtgacaatgc ctaacaataa aaatttcgat aaactataca tctgggggat tcatcacccg
  601 agctcaaaca aagagcagac aaaattgtat atccaagaat caggacgagt aacagtctca
  661 acagaaagaa gtcaacaaac agtaatccct aacatcggat ctagaccgtg ggtcaggggt
  721 caatcaggca ggataagcat atactggacc attgtaaaac ctggagatat tctaatgata
  781 aacagtaatg gcaacttagt tgcaccgcgg ggatatttta aattgagaac agggaaaagc
  841 tctgtaatga gatcagatgc acccataggc acttgtgtgt ctgaatgtat tacaccaaat
  901 ggaagcatcc ccaacgacaa accatttcaa aatgtgaaca aagttacata tggaaaatgc
  961 cccaagtata tcaggcaaaa cactttaaag ctggccactg ggatgaggaa tgtaccagaa
1021 aagcaaatca ga
```

DEFINITION Influenza A virus (A/eq/Arundel/91/(H3N8)) HA1 gene for HAY subunit of haemagglutinin, genomic RNA.

```
ACCESSION X85085; SEQ ID NO: 43
      1 atgaagacaa ccattatttt gatactactg acccattggg tctacagtca aaacccaacc
    61 agtggcaaca acacagccac attatgtctg ggacaccatg cagtagcaaa tggaacattg
  121 gtaaaaacaa taactgatga ccaaattgag gtgacaaatg ctactgaatt agttcagagc
  181 atttcaatag ggaaaatatg caacaactca tatagagttc tagatggaag aaattgcaca
  241 ttaatagatg caatgctagg agacccccac tgtgatgtct ttcagtatga gaattgggac
  301 ctcttcatag aaagaagcag cgctttcagc aattgctacc catatgacat ccctgactat
  361 gcatcgctcc ggtccattgt agcatcctca ggaacattag aattcacagc agagggattc
  421 acatggacag gtgtcactca aaacggaaga agtggagcct gcaaaagggg atcagccgat
  481 agtttcttta gccgactgaa ttggctaaca aaatctggaa attcttaccc catattgaat
  541 gtgacaatgc ctaacaataa aaatttcgat aaactataca tctgggggat tcatcacccg
  601 agctcaaaca aagagcagac aaaattgtac atccaagaat caggacgagt aacagtctca
  661 acaaaaagaa gtcaacaaac aataatccct aacatcggat ctagaccgtg ggtcaggggt
  721 caatcaggca ggataagcat atactggacc attgtaaaac ctggagatat cctaatgata
  781 aacagtaatg gcaacttagt tgcaccgcgg ggatatttta aattgagaac agggaaaagc
  841 tctgtaatga gatcagatgc acccatagac atttgtgtgt ctgaatgtat tacaccaaat
  901 ggaagcatcc ccagcgacaa accatttcaa aatgtaaaca aagttacata tggaaaatgc
  961 cccaagtata tcaggcaaaa cactttaaag ctggccactg ggatgaggaa tgtaccagaa
1021 aagcaaatca ga
```

DEFINITION Influenza A virus (A/equine/Suffolk/89(H3N8)) HA gene for haemagglutinin.

```
ACCESSION X68437; SEQ ID NO: 44
      1 ctgtcaatca tgaagacaac cattattttg atactactga cccattgggt ctacagtcaa
    61 aacccaacca gtggcaacaa cacagccaca ttatgtctgg gacaccatgc agtagcaaat
  121 ggaacattgg taaaaacaat aactgatgac caaattgagg tgacaaatgc tactgaatta
  181 gttcagagca tttcaatagg gaaaatatgc aacaactcat atagggttct agatggaaga
  241 aattgcacat taatagatgc aatgctagga gacccccact gtgatgtttt tcagtatgag
  301 aattgggacc tcttcataga aagaagcagc gctttcagca attgctaccc atatgacatc
  361 cctgactatg catcgctccg gtccattgta gcatcctcag gaacattaga attcacagca
  421 gagggattca catggacagg tgtcactcaa aacggaagaa gtggagcctg caaaagggga
  481 tcagccgata gtttctttag ccgactgaat tggctaacaa aatctggaaa ttcttacccc
  541 atattgaatg tgacaatgcc taacaataaa aatttcgata aactatacat ctgggggatt
  601 catcacccga gctcaaacaa agagcagaca aaattgtata tccaagaatc aggacgagta
  661 acagtctcaa cagaaagaag tcaacaaaca gtaatcccta acatcggatc tagaccgtgg
  721 gtcaggggtc aatcaggcag gataagcata tactggacca ttgtaaaacc tggagatatt
  781 ctaacgataa acagtaatgg caacttagtt gcaccgcggg gatattttaa attgagaaca
  841 gggaaaagct ctgtaatgag atcagatgca cccatagaca cttgtgtgtc tgaatgtatt
  901 acaccaaatg gaagcatccc caacgacaaa ccatttcaaa atgtgaacaa agttacatat
  961 ggaaaatgcc ccaagtatat caggcaaaac actttaaagc tggccaccgg gatgaggaat
1021 gtaccagaaa agcaaatcag aggaatcttt ggagcaatag cgggattcat agaaaacggc
1081 tgggaaggaa tggttgatgg gtggtatgga ttccgatatc aaaactcgga aggaacagga
1141 caagctgcag atctaaagag cactcaagca gccatcgacc agatcaatgg aaaattaaac
1201 agagtgattg aaaggaccaa tgagaaattc catcaaatag agaaggaatt ctcagaagta
1261 gaagggagaa tccaggattt ggagaagtat gtagaagaca ccaaaataga cctatggtcc
1321 tacaatgcag aattgctggt ggctctagaa aatcaacata caattgactt aacagatgca
1381 gaaatgaata aattattcga gaagactagg cgccagttaa gagaaaacgc ggaagacatg
1441 ggaggtggat gtttcaagat ttaccacaaa tgtgataatg catgcattgg atcaataaga
1501 aatgggacat atgaccatta catatacaga gatgaagcat taaacaaccg atttcaaatc
1561 aaaggtgttg agttgaaatc aggctacaaa gattggatac tgtggatttc attcgccata
1621 tcatgcttct taatttgcgt tgttctattg ggtttcatta tgtgggcttg ccaaaaaggc
1681 aacatcagat gcaacatttg catttgagta aactgatagt taaaaacacc cttgtttcta
1741 ct
```

DEFINITION Influenza A virus (A/equine/Newmarket/1/77(H7N7)) gene for haemagglutinin.

```
ACCESSION X62554; SEQ ID NO: 45
      1 nnnnnnnnnn nnnnnnnnna aatgaacact cagattctaa tattagccat ttcggcattc
    61 ctctgtgtac gtgcagataa aatctgccta ggacatcatg ctgtgtctaa tggaaccaaa
  121 gtagacaccc ttactgaaaa gggaatagaa gtcgtcaatg caacagaaac agttgaacaa
  181 aaaaacatcc ccaagatctg ctcaaaaggg aaacagacta ttgaccttgg tcaatgtgga
  241 ttactaggga ccactattgg tcccccccaa tgcgaccaat ttcttgaatt ctctgctaat
  301 ttaataattg agagaagaga aggtgatgat atttgttatc caggcaaatt tgacaatgaa
  361 gaaacattga gacaaatact cagaaaatcc ggaggaatta aaaaggagaa tatgggattc
  421 acatataccg gagtgagaac caatggagag actagcgcct gtagaaggtc aagatcttcc
  481 ttttatgcag aaatgaaatg gctcctatct aacacagaca atggggtatt cccacaaatg
  541 acaaaatcct acaagaacac taagaaggag ccagctctga taatctgggg aatccaccac
  601 tcaggatcaa ctgctgaaca gactagattg tatggaagtg gaaacaagtt gataacagtt
  661 tggagttcca aataccaaca atcttttgcc ccaaaccctg gaccaaggcc gcaaatgaat
  721 ggccaatcag gaagaattga cttttactgg ctgatgttag atcccaatga tactgttaat
  781 ttcagtttta atggggcctt tatagcacct gaccgcgcca gttttctaag aggtaaatct
  841 ctaggaattc agagtgacgc acaacttgac aacaattgtg aaggtgaatg ttatcatatt
  901 ggaggtacca taattagcaa cttgcccttt caaaacatta atagcagagc aattgggaaa
  961 tgccccagat acgtaaagca aaaaagctta atgctagcaa ccggaatgaa aaatgttcct
1021 gaaaattcta cacacaaaca gttaactcat cacatgcgca aaaaaagagg tttatttggt
1081 gcaatagcag gatttattga aaatggatgg gaaggattaa tagatggatg gtatggatac
1141 agacatcaga atgcacaagg agaaggaact gctgcagact acaaaagtac acaatctgct
1201 gtcaatcaaa taaccgggaa attaaacaga ctaatagaaa aaaccaacca gcaatttgaa
1261 ctaatagata atgaattcaa tgaaatagaa aagcaaattg gcaatgttat taactggact
1321 agagattcta tcatcgaaat atggtcatat aatgcagaat tcctcgtggc agtggagaat
1381 caacacacta ttgatttaac tgattcagag atgaacaaat tatatgaaaa ggtaagaaga
1441 cagctgagag aaaatgctga ggaagatggt aatggctgtt ttgaaatatt tcaccaatgt
1501 gacaatgatt gcatggccag cattagaaac aatacatatg atcataaaaa atacagaaag
1561 gaggcaatac aaaacagaat tcagattgat gcagtaaagt tgagcagcgg ttacaaagaa
1621 ataatacttt ggtttagctt cggggcatca tgtttcttat ttcttgccat tgcaatggtt
1681 cttgctttca tatgcataaa aaatggaaac atgcggtgca ctatttgtat ataagtttga
1741 aaaaacaccc ttgtttctan n
```

DEFINITION Influenza A virus HA partial gene for haemagglutinin, genomic RNA, strain A/equine/Newmarket-Bob Champion/89(H3N8).

```
ACCESSION AJ223193; SEQ ID NO: 46
      1 agtcaaaacc caaccagtgg caacaacaca gccacattat gtctgggaca ccatgcagta
    61 gcaaatggaa cattggtaaa aacaataact gatgaccaaa ttgaggtgac aaatgctact
  121 gaattagttc agagcatttc aatagggaaa atatgcaaca actcatatag ggttctagat
  181 ggaagaaatt gcacattaat agatgcaatg ctaggagacc cccactgtga tgtttttcag
  241 tatgagaatt gggacctctt catagaaaga agcagcgctt tcagcaattg ctacccatat
  301 gacatccctg actatgcatc gctccggtcc attgtagcat cctcaggaac attagaattc
  361 acagcagagg gattcacatg gacaggtgtc actcaaaacg gaagaagtgg agcctgcaaa
  421 aggggatcag ccgatagttt ctttagccga ctgaattggc taacaaaatc tggaaattct
  481 taccccatat tgaatgtgac aatgcctaac aataaaaatt tcgataaact atacatctgg
  541 gggattcatc acccgagctc aaacaaagag cagacaaaat tgtatatcca agaatcagga
  601 cgagtaacag tctcaacaga aagaagtcaa caaacagtaa tccctaacat cggatctaga
  661 ccgtgggtca ggggtcaatc aggcaggata agcatatact ggaccattgt aaaacctgga
  721 gatattctaa tgataaacag taatggcaac ttagttgctc cgcggggata ttttaaattg
  781 agaacaggga aaagctctgt aatgagatca gatgcaccca tagacacttg tgtgtctgaa
  841 tgtattacac caaatggaag catccccaac gacaaaccat ttcaaaatgt gaacaaagtt
  901 acatatggaa aatgccccaa gtatatcagg caaaacactt taaagctggc cactgggatg
  961 aggaatgtac cagaaaagca aatcagagga atctttggag caatagaggg attcatagaa
1021 aacggctggg aaggaatggt tgatgggtgg tatggattcc gatatcaaaa ctcggaagga
1081 acaggacaag ctgcag
```

DEFINITION Influenza A virus (A/Equine/NewMarket/D64/79(H3N8)) gene for hemagglutinin precursor, partial cds.

```
ACCESSION D30677; SEQ ID NO: 47
      1 ctgtcaatca tgaagacaac cattattttg atactactga cccattgggt ctacagtcaa
    61 aacccaacca gtggcaacaa cacagccaca ctatgtctgg gacaccatgc agtagcaaat
  121 ggaacattgg taaaaacaat aactgatgac caaattgagg tgacaaatgc cactgaatta
  181 gttcagagca cttcaatagg gaaaatatgc aacaacccat atagggttct agatggaaga
  241 aactgcacat taatagatgc aatgctagga gatccccact gtgatgtttt tcagtatgag
  301 aattgggacc tcttcataga aagaagcagc gctttcagca attgctaccc atatgacatc
  361 cctgactatg catcgctccg gtctattgtg gcatcttcag gaacattaga attcacagca
  421 gagggattca catggacagg tgtcactcaa aacggaagaa gtggcgcctg cagaagggga
  481 tcagccgata gtttctttag ccgactgaat tggctaacaa aatctggaga ttcttacccc
  541 acattgaatg tgacaatgcc taacaataac aatttcgata aactatacat ctgggggatc
  601 catcacccga gcacaaacaa tgagcagaca aaattgtatg tccaagaatc agggcgagta
  661 acagtctcaa caaaaagaag tcaacaaaca ataatcccca acatcggatc tagaccgtgg
  721 gtcaggggtc aaccaggcag gataagcata tattggacca ttgtgaaacc tggagatatc
  781 ctaatgataa acagtaatgg caacttagtt gcaccgcggg gatatttcaa aatgcgaaca
  841 ggaaaaagct ctataatgag atcagatgca cccatagaca cttgtgtgtc cgagtgtatt
  901 acaccaaatg gaagcatccc caacgacaaa ccatttcaaa atgtgaacaa agttacatat
  961 gggaaatgcc ccaagtatat caagcagaat actttgaagc tggccactgg gatgaggaat
1021 gtaccagaaa agcaaatcag aggaatcttt ggagcaatag cgggattcat agaaaatggc
1081 tgggag
```

DEFINITION Influenza A virus (A/eq/Newmarket/1/77(H7N7)) matrix proteins M1 and M2 (M) gene, complete cds.

```
ACCESSION AF001686; SEQ ID NO: 48
    1 atgagtcttc tgaccgaggt cgaaacgtac gttctctcta tcgtaccatc aggccccctc
  61 aaagccgaga tcgcgcagag acttgaagat gtctttgcag gaaagaacac cgatcttgag
121 gcactcatgg aatggctaaa gacaagacca atcctgtcac ctctgactaa ggggatttta
181 ggatttgtgt tcacgctcac cgtgcccagt gagcgaggac tgcagcgtag acgctttgtc
241 caaaatgccc ttaatgggaa cggagatcca aacaacatgg acagagcagt aaaactgtac
301 aggaagctta aaagggaaat aacattccat ggggcaaaag aggtggcact cagctattcc
361 actggtgcac tagccagctg catgggactc atatacaaca gaatggggac tgtgacaacc
421 gaagtggcat ttggcctggt atgcgccaca tgtgaacaga ttgctgattc ccagcaccga
481 tctcacagac agatggtgac aacaaccaac ccactaatca gacacgagaa cagaatggta
541 ctagccagta ccacagctaa agccatggag cagatggcag ggtcgagtga gcaggcagca
601 gaggccatgg aggttgctag tcaggccagg cagatggtgc aggcaatgag aaccattggg
661 acccacccta gctccagtgc cggtttgaaa aatgatcttc ttgaaaattt gcaggcctac
721 cagaaacgga tgggagtgca aatgcagcga ttcaagtgat cctctcgtta ttgcagcaag
781 tatcattggg atcttgcact tgatattgtg gattcttgat cgtcttttct tcaaatgcat
841 ttatcgtcgt cttaaatacg gtttgaaaag agggccttct acggaaggag tacctgagtc
901 tatgagggaa gaatatcggc aggaacagca gagtgctgtg gatgttgacg atggtcattt
961 tgtcaacata gagctggagt aa
```

DEFINITION Influenza A virus (A/eq/Newmarket/79(H3N8)) matrix proteins M1 and M2 (M) gene, complete cds.

```
ACCESSION AF001675; SEQ ID NO: 49
    1 atgagtcttc taaccgaggt cgaaacgtac gttctctcta tcgtaccatc aggccccctc
  61 aaagccgaga tcgcgcagag acttgaagat gtctttgcag ggaagaacac cgatcttgag
121 gcactcatgg aatggctaaa gacaagacca atcctgtcac ctctgactaa agggatttta
181 ggatttgtgt tcacgctcac cgtgcccagt gagcgaggac tgcagcgtag acgctttgtc
241 caaaatgccc ttagtggaaa cggagatcca aacaacatgg acagagcagt aaaactgtac
301 aggaagctta aaagagaaat aacattccat ggggcaaaag aggtggcact cagctattcc
361 actggtgcac tagccagctg catgggactc atatacaaca gaatggggac tgtgacaacc
421 gaagtggcat ttggcctggt atgcgccaca tgtgaacaga ttgctgattc ccagcatcga
481 tctcacaggc agatggtgac aacaaccaac ccactaatca gacatgaaaa cagaatggta
541 ctagccagta ccacagctaa agccatggag cagatggcag ggtcgagtga gcaggcagca
601 gaggccatgg aggttgctag taaggccagg cagatggtac aggcaatgag gaccattggg
661 acccacccta gctccagtgc cggtttgaaa gatgatcttc ttgaaaattt gcaggcctac
721 cagaaacgga tgggagtgca aatgcagcga ttcaagtgat cctctcgtta ttgcagcaag
781 tatcattggg atcttgcact tgatattgtg gattcttgat cgccttttct tcaaattcat
841 ttatcgtcgc cttaaatacg gtttgaaaag agggccttct acggaaggag tacctgagtc
901 tatgagggaa gaatatcggc aggaacagca gaatgctgtg gatgttgacg atggtcattt
961 tgtcaacata gagctggagt aa
```

DEFINITION Influenza A virus (A/eq/Newmarket/1/77(H7N7)) nonstructural proteins NS1 and NS2 (NS) gene, complete cds.

```
ACCESSION AF001663; SEQ ID NO: 50
    1 atggattcca acactgtgtc aagctttcag gtagactgtt ttctttggca tgtccgcaaa
  61 cgatttgcag accaagaact gggtgatgcc ccattccttg accggcttcg ccgagaccag
121 aagtccctaa aaggaagagg cagcactctt ggtctggaca tcgaaacagc cactcatgca
181 ggaaagcaga tagtggagcg aattctggaa gaggaatcag atgaggcact taaaatgacc
241 atagcctctg ttcctacttc acgctactta actgacatga ctcttgatga gatgtcaaga
301 gactggttca tgctcatgcc caagcagaaa gtaacaggct ccctatgtat aaggatggac
361 caagcaatca tggataagaa catcatacta aaagcaaact ttagtgtgat tttcgaaagg
421 ctggagacac taatactact tagagctttc accgaagaag gagcagtcgt tggcgaaatt
481 tcaccattgc cttctcttcc aggacatact aatgaggatg tcaaaaatgc aattggggtc
541 ctcatcggag gacttaaatg gaatgataac acagttagag tctctgaaac tctacagaga
601 ttcgcttgga gaagcagtca tgagaatggg agaccttcat tccctccaaa gcagaaacga
661 aaaatggcga gaacaattga gtcagaagtt tgaagaaata aggtggttga ttgaagaagt
721 gagacataga ttgaaaaata cagaaaatag ttttgaacaa ataacattta tgcaagcctt
781 acaactattg cttgaagtag aacaagagat aagaactttc tcgtttcagc ttatttaa
```

DEFINITION Influenza A virus (A/eq/Newmarket/D63/79(H3N8)) nonstructural proteins NS1 and NS2 (NS) gene, complete cds.

```
ACCESSION AF001662; SEQ ID NO: 51
    1 atggattcca acactgtgtc aagctttcag gtagactgtt ttctttggca tgtccgcaaa
  61 cgatttgcag accaagaact gggtgatgcc ccattccttg accggcttcg ccgagaccag
121 aagtccctaa aaggaagagg cagcactctt ggtctggaca tcgaaacagc cactcgtgca
181 ggaaagcaga tagtggagcg gattctggaa gaggagtcag atgaggcact taaaatgacc
241 attgcctctg ttcctgcttc acgctactta actgacatga ctcttgatga gatgtcaaga
301 gactggttca tgctcatgcc caagcagaaa gtaacaggct ccctatgtat aaggatggac
361 caggcaatca tggataagaa catcatacta aaagcaaact ttagtgtgat tttcgaaagg
421 ctggagacac taatactact tagagccttc accgaagaag gagcagtcgt tggcgaaatt
481 tcaccattgc cttctcttcc aggacatact aatgaggatg tcaaaaatgc aattggggtc
541 ctcatcggag gacttaaatg gaatgataat acagttagag tctctgaaac tctacagaga
601 ttcgcttgga gaagcagtca tgagaatggg agaccttcat tccctccaaa gcagaaacga
661 aaaatggcga gaacaattga gccagaagtt tgaagaaata agatggttga ttgaagaagt
721 gcgacataga ttgaaaaata cagaaaatag ttttgaacaa ataacattta tgcaagcctt
781 acaactattg cttgaagtag aacaagagat aagaactttc tcgtttcagc ttatttaa
```

### EXAMPLE 6

### Efficacy of Inactivated Influenza Virus Vaccine in Canines

Vaccine active ingredient (AI) is produced either in 9-11 day-old embryonated chicken eggs or in continuous cell cultures such as MDCK cells.

Egg Production Harvest and Purification. Vaccine AI production in embryonating chicken eggs is prepared by inoculation 0.1-0.25 ml of virus suspension containing 100-1000 EID50 (egg infectious dose) into the allantoic cavity. Allantoic fluid is harvested after incubation at 33-37° C. for 2-6 days and pooled. The virus pool is clarified by centrifugation at 1500-4000 g for 5-15 minutes and filtered through a 20 μm filter (1). The virus maybe concentrated by ultra-filtration using a 300 KDa membrane with a maximum concentration factor of 20 (2).

Further the concentrated virus is optionally purified by zonal ultra-centrifugation on a sucrose gradient followed by filtration of the virus containing fraction through a 0.5 μm membrane. This step can be repeated. The virus containing fraction is diluted in a suitable buffer such as a phosphate buffer (3).

Further purification consists of treatment with Tween/ether at 0.1% final concentration (w/v)) and 50% final concentration (w/w), respectively. The resulting solution is centrifuged and the aqueous phase is collected. This step can be repeated. The aqueous phase is treated with nitrogen to remove residual ether (4).

Cell Culture Production Harvest and Purification. Active ingredient is prepared in cells (e.g. MDCK, BHK, PerC6 cells) by inoculation at a rate of MOI of 0.001-1 TCID50. Culture is harvested after incubation at 35-37 C for 48-96 hrs when cytopathic effect (CPE) is approximately 50-90%. Culture is sonicated and centrifuged at 1500-4000 g for 5-15 minutes (1). The virus is concentrated by ultra-filtration using a 300 KD membrane with a max. concentration factor of 20 (2).

The virus is optionally purified by exclusion chromatography (Sepharose 6 fast flow)(3).

Inactivation. Virus (fraction 1, 2, 3 or 4) are inactivated by addition of formalin or beta-propiolactone to a final concentration of 0.05% (w/v) or 0.01% (w/v), respectively and the suspension is held respectively at 5° C. for 16-18 hours or at 20° C. for 25-36 hours with continuous agitation.

Active ingredient inactivation is confirmed by inoculation into embryonated chicken eggs or onto MDCK cells.

Preparation of Final Vaccine. Vaccine consists of the inactivated influenza virus and an adjuvant such as aluminium hydroxide, aluminum phosphate, Carbomer or oil-in-water emulsion, which may contain other immunostimulating components such as CpG (1-100 ug/dose). Vaccine typically contains 15-50 μg of hemagglutinin measured by single radial-immunodiffusion (SRD) or >400 haemagglutination Units

The vaccine is administered either by subcutaneous or intramuscular route. Two doses are administered at an interval of 2-5 weeks to pups from 4 weeks of age. Duration of immunity: 6-12 months after a primary vaccination course.

Vaccination/Challenge Protocol. Dogs (less than 4 weeks old) are vaccinated with 2 doses of inactivated vaccine at 3-4 weeks interval via the subcutaneous route. Blood samples are collected on the day of the first and second vaccination and 2-4 weeks after second vaccination to determine the levels of anti-influenza virus specific antibodies using virus neutralization, hemagglutination inhibition, ELISA or Single Radial Heamolysis (SRH) tests.

Two to 4 weeks after 2^{nd} immunization dogs are challenged with a virulent strain of influenza H3N8 to determine efficacy of the vaccines.

Vaccinated and control dogs are challenged by the following route:

By spray: animals are placed into an enclosed container or a mask is placed on the nose of the dogs and the virus suspension is aerosolized to generate 1-100 μM droplets. Animals are kept in the aerosolization chambers for 30 minutes to allow inhalation of the aerosol.

Alternativelly thy can be challenged by one the following routes:

Intra-nasally: 0.5 ml containing 10⁵⁻⁸ EID50 of challenge virus is dropped to each nostril by the aid of dropper.

Intra-tracheally: 1-2 ml containing 10⁵⁻⁸ EID₅₀ virus is directly instilled into the trachea.

Orally: 5-10 ml containing 10⁵⁻⁸ EID50 of challenge virus is orally administered.

Animals are observed daily for 14 days following challenge for clinical signs including fever, cough, nasal, ocular discharge, respiratory distress, anorexia and lethargy. In addition, groups of animals are euthanized and evaluated for pathological findings of pulmonary and pleural hemorrhage, tracheitis, bronchitis, broncholilitis, and bronchopneumonia.

Tracheal swabs are collected from all animals post challenge days 1-14 for virus isolation.

Presence or absence of viral antigens in respiratory tissues is evaluated by immunohistochemistry on days 3, 7 and 10 post-challenge.

Blood samples are collected on days 7 and 14 post-challenge and are analyzed for the presence of anti-influenza H3N8 virus specific antibody.

### EXAMPLE 7

### Vaccination of Dogs with Equine Influenza Vaccines to Induce a Humoral Immune Response Against CIV

A study was conducted in which 35 dogs were randomly allocated into four groups of 9, 9, 9 and 8 dogs. None of the dogs had detectable antibodies to CIV, as determined by HI tests (below) at the commencement of the study. One group was immunized with vCP1529 (canarypox recombinant expressing HA gene from Kentucky equine influenza, A/eq/Kentucky/94) 10^{5,7} TCID₅₀/1 ml dose, which was administered subcutaneously, and two groups were immunized with vCP2242 (canarypox recombinant expressing HA gene from A/eq/Ohio/03), which was administered to one group subcutaneously (10^{5,7} TCID₅₀/1 ml dose) and to one group transdermally (10^{5,7} TCID₅₀/0.25 ml dose). Vaccinates received 2 doses of vaccine on Day 0 and Day 28. The fourth group was unvaccinated and served as a control. All dogs were examined for local reactions at the site of inoculation and for general clinical signs.

Blood samples were collected for serology on days −1, 7, 13, 21, 28, 35, and 42 for all groups. A hemagglutination inhibition (HI) test was performed as described previously (Karaca et al., 2007). Briefly, sera were separated and were heat inactivated (56° C. for 30 minutes). Sera were then treated with potassium periodate followed by incubation with turkey RBCs to remove nonspecific HAs and inhibitors. Four hemagglutination units of antigen were added to serial 2-fold dilutions of treated sera and incubated at 18° to 22° C. for 30 minutes. Turkey RBCs were added, and plates were incubated for an additional 30 minutes. Wells were then examined for evidence of hemagglutination. The lowest dilution of serum tested was 1:8, and antibody titers corresponding to the reciprocal of the highest dilution that inhibited hemagglutination were presented as geometric mean titers (GMTs).

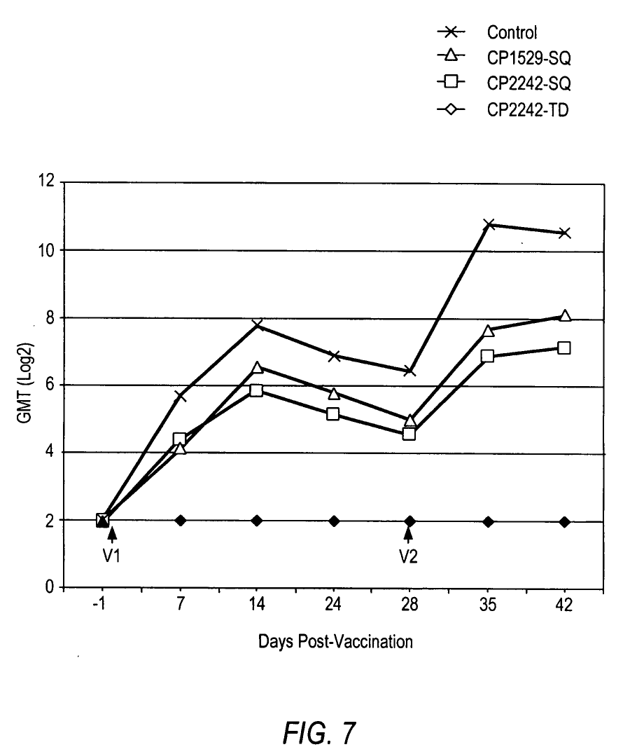

All dogs tolerated the inoculations well. None of the dogs had a systemic or local adverse reaction, regardless of the inoculum or route of administration. Antibody responses of dogs as measured by HI tests are shown in FIG. 7. On day −1, all dogs were seronegative (antibody titres <8). Control dogs remained seronegative throughout the duration of the study. All inoculated dogs had detectable antibodies against CIV on days 13, 21, and 28 after the initial inoculation. Although there was no significant difference in antibody titers between subcutaneously inoculated groups, antibody titres of the transdermally inoculated group were significantly higher than the others after the first and second inoculations. (repeated measures ANOVA, p<0.01).

### EXAMPLE 8

### Vaccination of Dogs with Equine Influenza Vaccines and Challenge with a Virulent Canine Influenza Isolate

A study was conducted in which 33 dogs were randomly allocated to one of two groups. Dogs of group 1 (n=16) were not inoculated and served as a control. Dogs of group 2 (n=17) were inoculated subcutaneously on day 0 and day 28 with 1057 TCID₅₀ of CP2242 (canarypox recombinant expressing HA gene from A/eq/Ohio/03). All dogs were challenged on day 43 with 10^{7,0} TCID₅₀ of CIV-NY05 first by syringe 0.5 ml directly into the nares followed with challenge by aerosolization into a closed chamber for 5-15 minutes, with oxygen supplied using a commercially available nebulizer. All dogs were clinically observed pre and post challenge according to the timeline in Table 2.

TABLE 2
Timeline
(VI = virus isolation;
Bb = Bordetella bronchiseptica isolation)

| Study | | |
|---|---|---|
| Day | DPC* | ACTIVITY |
| D-4 | N/A | Baseline injection site observations |
| | | Nasal swab for VI and Bb |
| | | Baseline Blood for serology |
| D0 | | Vaccination #1 |
| D1-4 | | Injection site observations |
| D7 | | Injection site observations |
| | | Weigh |
| D16-18 | | Baseline Injection site observations |
| D21 | | Baseline Injection site observations |
| | | Weigh |
| | | Nasal swabs for VI and Bb |
| | | Baseline Blood Collection for serology |
| | | Vaccination #2 |
| D22-25 | | Injection site observations |
| D28 | | Injection site observations |
| | | Baseline Clinical observations |
| | | Nasal swabs for VI and Bb |
| | | Baseline Blood Collection for serology |
| D42 | | Baseline Clinical Observations (Weigh) |
| | | Nasal swabs for VI and Bb |
| | | Baseline Blood collection for serology |
| D43 | | Challenge |
| D44-48 | 1-5 | Clinical observations |
| | | Nasal swabs for VI and Bb |
| D49 | 6 | Clinical observations (Weigh) |
| | | Nasal swabs for VI and Bb |
| D50 | 7 | Clinical observations |
| | | Nasal swabs for VI and Bb |
| | | Blood for serology |
| D52-53 | 9-10 | Clinical observations |
| | | Nasal swabs for VI and Bb |
| D57 | 14 | Clinical observations (Weigh) |
| | | Nasal swabs for VI and Bb |
| | | Blood for serology |
| | | Euthanasia- End of Study |

*Days Post-Challenge

The clinical signs that were recorded as absent or present include cough (spontaneous or inducible by mild tracheal palpation), ocular/nasal discharge (serous or purulent), rectal temperature (° F.), depression/lethargy, dyspnea, and weight loss.

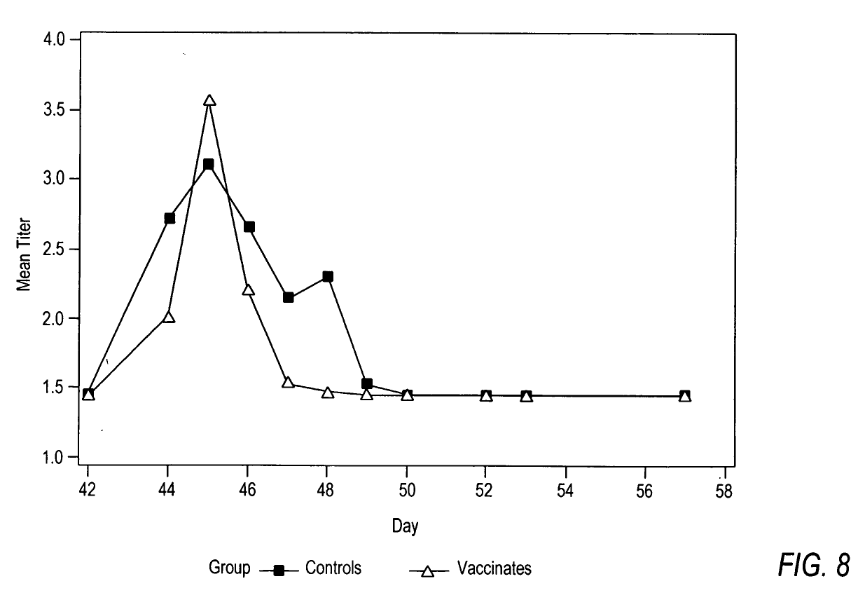

All dogs were evaluated for CIV viral load, body temperature, HI antibody responses to CIV HA antigen, and virus shedding. To determine CIV viral loads, nasal swabs were collected from all animals on days −4, 21, 28, and 42 pre-challenge samples and on days 44-50, 52, 53 and 57 (1-7,9,10 and 14 days post-challenge). Levels of virus were determined using a virus titration assay on Madine Darby canine kidney (MDCK) cells and the titer results were expressed as TCID₅₀. FIG. 8 illustrates describes the mean viral titers from swab extracts in controls and vaccinates. In this example, a TCID₅₀ of 1.45 is the minimum detectable titer, therefore a titer of 1.45=no virus detected. The vaccinated animals stop shedding at Day 47 whereas control animals shed until Day 49. Controls had a significantly higher titer than vaccinates on Day 44 (p<0.0001), Day 46 (p=0.0016), Day 47 (p=0.0027) and Day 48 (p<0.0001).

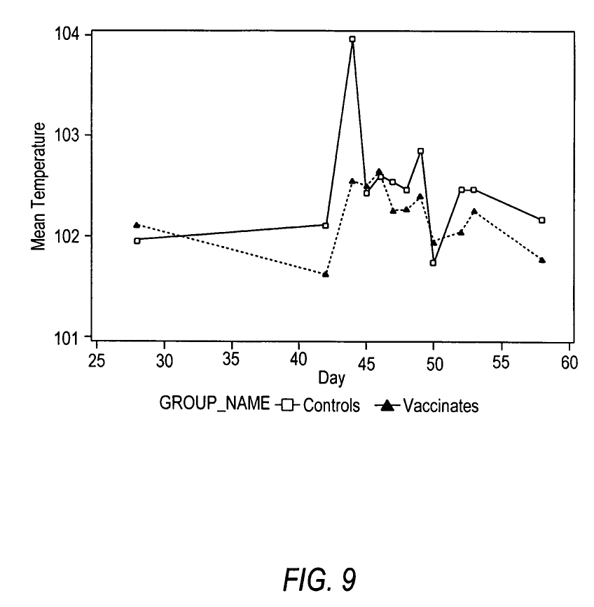

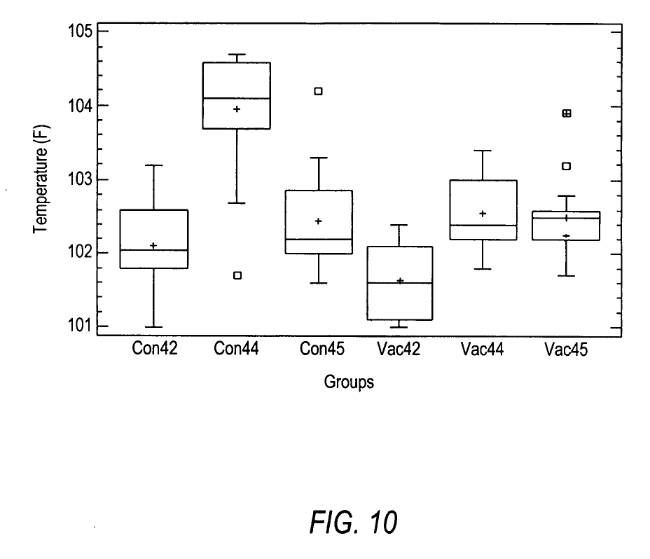

Body temperatures of dogs were evaluated throughout the study. The mean body temperatures of dogs vaccinated with vCP2242 were lower than the mean body temperatures of control dogs (FIGS. 9 and 10).

FIG. 9 shows the mean temperature over time from Day 28 to Day 57. Controls show an increase from a 102° F. baseline to 104° F., 24 hours after challenge whereas vaccinate temperature increase from 102° F. to 102.5° F. (statistically significant difference on that day with p<0.0001). Controls also have a significantly higher mean body temperature on day 49 (p=0.048).

FIG. 10 is a boxplot for Days 42 (1 day pre-challenge), 44 and 45 (1 and 2 days post-challenge).

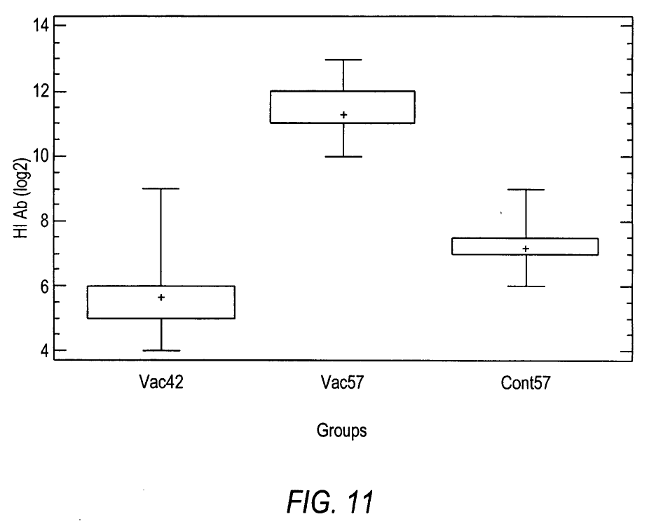

FIG. 11 shows the HI titers on Day 42 (1 day pre-challenge) and Day 57 (14 days post-challenge). A virulent CIV-NY05 challenge boosts the HI titers of dogs vaccinated with vCP2242. Although unvaccinated animals seroconverted after challenge, their mean Day 57 HI titer was significantly lower than vaccinates. This is an evidence of the efficient priming provided by the vaccine.

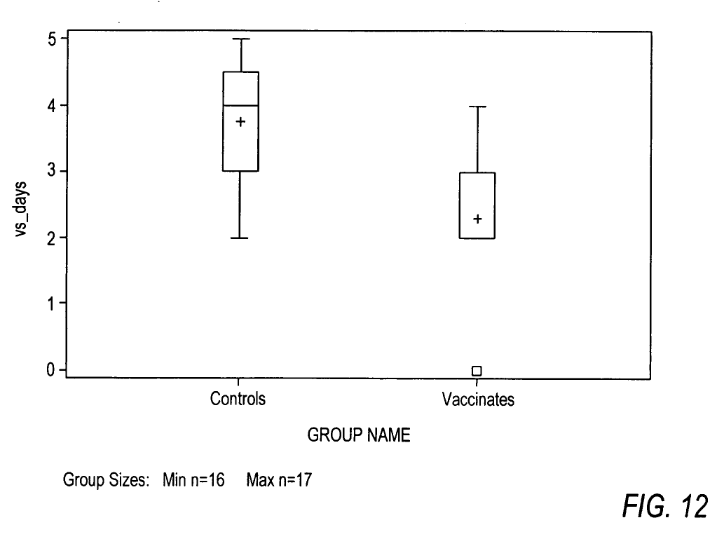

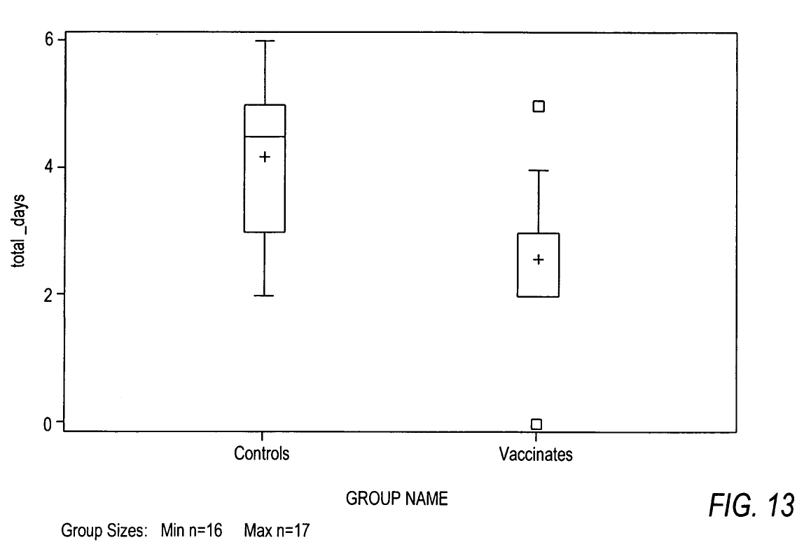

On average, dogs vaccinated with HA antigen of A2/equine/Ohio/03 (vCP2242) followed by challenge at day 43 with CIV-NY05 isolate demonstrated a decreased number of shedding days as compared to control dogs (FIG. 12) as well as a decreased duration of virus shedding in the nasal cavity (FIG. 13).

FIG. 12 show the number of shedding days illustrating the decrease in number of shedding days in the vaccinates vs. controls.

FIG. 13 shows the decrease in the duration of virus shedding in the vaccinates.

The invention is further described by the following numbered paragraphs:

1. A method of eliciting an immune response against influenza in a canine, comprising administering a formulation comprising an avipox expression vector comprising a polynucleotide encoding an influenza antigen, epitope or immunogen and a pharmaceutically or veterinarily acceptable carrier, excipient or vehicle in an effective amount for eliciting an immune response.

2. A method of inducing an immune response against influenza in a canine, comprising administering a formulation comprising an avipox expression vector comprising a polynucleotide encoding an influenza antigen, epitope or immunogen and a pharmaceutically or veterinarily acceptable carrier, excipient or vehicle in an effective amount for inducing an immune response.

3. The method of paragraph 1 or 2, wherein the formulation further comprises an adjuvant.

4. The method of any one of paragraphs 1 to 3, wherein the influenza antigen, epitope or immunogen is a hemagglutinin, matrix protein, membrane protein, neuraminidase, nonstructural protein, nucleoprotein, polymerase or any fragment thereof.

5. The method of any one of paragraphs 1 to 4, wherein the influenza antigen, epitope or immunogen is isolated from a canine infected with influenza.

6. The method of paragraph 5 wherein the influenza antigen, epitope or immunogen is isolated from the broncho alveolar lavage and/or lung tissues of the canine.

7. The method of any one of paragraphs 1 to 4, wherein the influenza antigen, epitope or immunogen is isolated from an equine influenza.

8. The method of paragraph 7, wherein the equine influenza is an Ohio equine influenza, a Kentucky equine influenza or a Newmarket equine influenza.

9. The method of any one of paragraphs 1 to 8, wherein the avipox expression vector is an attenuated avipox expression vector.

10. The method of paragraph 9, wherein the avipox expression vector is a canarypox vector.

11. The method of paragraph 10, wherein the canarypox vector is ALVAC.

12. The method of paragraph 10 or 11, wherein the influenza antigen, epitope or immunogen is a hemagglutinin.

13. The method of paragraph 12, wherein the hemagglutinin is H3.

14. The method of paragraph 12 or 13, wherein the canarypox vector is CP1529 or CP1533.

15. A method of eliciting an immune response against influenza in a canine, comprising administering a formulation comprising an inactivated influenza vaccine and a pharmaceutically or veterinarily acceptable carrier, excipient or vehicle in an effective amount for eliciting an immune response.

16. A method of inducing an immune response against influenza in a canine, comprising administering a formulation comprising an inactivated influenza vaccine and a pharmaceutically or veterinarily acceptable carrier, excipient or vehicle in an effective amount for inducing an immune response.

17. The method of paragraph 15 or 16, wherein the formulation further comprises an adjuvant.

18. The method of any one of paragraph 15 to 17, wherein the inactivated influenza vaccine is an inactivated canine influenza.

19. The method of any one of paragraphs 15 to 17, wherein the inactivated influenza vaccine is an inactivated equine influenza.

20. The method of paragraph 19, wherein the equine influenza is an Ohio equine influenza, a Kentucky equine influenza or a Newmarket equine influenza.

21. The method of any one of paragraphs 15 to 20, wherein the inactivated influenza vaccine is inactivated with formalin or beta-propiolactone.

22. The method of any one of paragraphs 17 to 21, wherein the adjuvant is aluminum hydroxide, aluminum phosphate, a carbomer or an oil-water-emulsion.

23. The method of any one of paragraphs 17 to 22, wherein the adjuvant further comprises CpG.

24. The method of any one of paragraphs 15 to 23, wherein the administration is subcutaneous or intramuscular.

25. A kit for performing any one of the methods of paragraphs 1 to 24 comprising the vaccine of any one of paragraphs 1 to * and instructions for performing the method of any one of paragraphs 1 to 24.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A Canidae vaccine comprising an effective amount of an expression vector that contains and expresses in vivo in a canine a polynucleotide encoding equine influenza antigen comprising equine influenza H3 operatively linked to a promoter and optionally to an enhancer, and a pharmaceutically or veterinarily acceptable carrier, excipient, or vehicle.

2. The vaccine of claim 1, wherein the polynucleotide comprises H3N8 codon optimized HA.

3. The vaccine according to claim 1, wherein the polynucleotide is isolated from a canine infected with equine influenza.

4. The vaccine according to claim 3, wherein the polynucleotide is isolated from a broncho alveolar lavage of the infected canine.

5. The vaccine according to claim 3, wherein the polynucleotide is isolated from lung tissue of the infected canine.

6. The vaccine according to claim 1, wherein the polynucleotide is isolated from an equine influenza virus isolate.

7. The vaccine according to claim 6, wherein the equine influenza isolate is selected from the group consisting of an Ohio equine influenza virus isolate, a Kentucky equine influenza virus isolate, a Newmarket equine influenza virus isolate or mixtures thereof.

8. The vaccine according to claim 6, wherein the polynucleotide is isolated from an Ohio equine influenza virus isolate.

9. The vaccine according to claim 6, wherein the polynucleotide is isolated from a Kentucky equine influenza virus isolate.

10. The vaccine according to claim 6, wherein the polynucleotide is isolated from a Newmarket equine influenza virus isolate.

11. The vaccine according to claim 1, wherein the expression vector is a poxvirus.

12. The vaccine according to claim 11, wherein the poxvirus is an avipox virus.

13. The vaccine according to claim 12, wherein the avipox virus is a canarypox virus.

14. The vaccine according to claim 13, wherein the canarypox virus is ALVAC.

15. The vaccine according to claim 13, wherein the canarypox virus is selected from CP1529, CP1533, or CP2242.

16. A method of vaccinating a Canidae comprising administering to said Canidae an effective amount of a vaccine comprising an effective amount of an expression vector that contains and expresses in vivo in a canine a polynucleotide encoding equine influenza antigen comprising equine influenza H3 operatively linked to a promoter and optionally to an enhancer, and a pharmaceutically or veterinarily acceptable carrier, excipient, or vehicle.

17. The method of claim 16, wherein the vaccine further comprises an adjuvant.

18. The method of claim 16, wherein the polynucleotide is isolated from an equine influenza virus isolate selected from the group consisting of an Ohio equine influenza virus isolate, a Kentucky equine influenza virus isolate, a Newmarket equine influenza virus isolate or mixtures thereof.

19. The method of claim 17, wherein the adjuvant is a cationic lipid containing a quaternary ammonium salt.

20. The method of claim 16, wherein the administering is subcutaneously or intramuscularly.


SEQUENCE LISTING (51 sequences)

SEQ ID NO: 1 - DNA, 3959 bp, Influenza A virus
gtattctaaactaggaatagatgaaattatgtgcaaaggagatacctttagatatggatctgatttatttggtttttcataatcataatctaacaacattttcactatactataccttcttgcacaagtcgccattagtagtatagacttatactttgtaaccatagtatactttagcgcgtcatcttcttcatctaaaacagatttacaacaataatcatcgtcgtcatcttcatcttcattaaagttttcatattcaataactttcttttctaaaacatcatctgaatcaataaacatagaacggtatagagcgttaatctccattgtaaaatatactaacgcgttgctcatgatgtacttttttttcattatttagaaattatgcattttagatctttataagcggccgtgattaactagtcataaaaacccgggatcgattctagactcgagcggccgccagtgtgatggatatctgcagaattcggctttggtccttactcaaatgcaaatgttgcacctgatgttgcctttttggcaagcccacataatgaaacccaatagaacaacgcaaattaagaagcatgatatggcgaatgaaatccacagtatccaatctttgtagcctgatttcaactcaacacttttgatttgaaatcggttgtttaatgcttcatctctgtatatgtaatggtcatatgtcccatttcttattgatccaatgcatgcattatcacatttgtggtaaatcttgaaacatccacctcccatgtcttccgcgttttctcttaactggcgcctagtcttctcgaataatttattcatttctgcatctgttaagtcaattgtatgttgattttctagagccaccagcaattctgcattgtaggaccataggtctattttggtgtcttctacatacttctccaaatcctggattctcccttctacttctgagaattccttttctatttgatggaatttctcattggtcctttcaatcactctgtttaattttccattgatctggtctatggctgcttgagtgctctttagatctgcagcttgtcctgttccttccgagttttgatatcggaatccataccacccatcaaccattccttcccagccgttttctatgaatcccgctattgctccaaagattcctctgatttgcttttctggtacattcctcatcccagtggccagctttaaagtgttttgcctgatatacttggggcattttccatatgtaattttgttcacattttgaaatggtttgtcgttggggatgcttccatttggtgtaatacattcagacacacaagtgtctatgagtgcatctgatctcattacagagcttttccctgttctcaatttaaaatatccccgcggtgcaactaagttgccattactgtttatcattagaatatctccaggttttacaatggtccagtatatgcttatcctgcctgattgacccctgacccacggcctagatccgatgttagggattactgtttgttgacttctttctgttgagactgttactcgtcctgattcttggatatataattttgtctgctctttgtttgagctcgggtgatgaatcccccagatgtatagtttatcgaaatttttattgttaggcattgtcacattcaatatggggtaagaatttccagattttgttagccaattcagtcggctaaagaaactatcggctgatccccttttgcaggctccacttcttccgttttgagtgacacctgtccatgtgaacccctctgctgtgaattctaatgttcctgaggatgctacaatggaccggagcgatgcatagtcagggatgtcatatgggtagcaattgctgaaagcgctgcttctttctatgaagaggtcccaattctcatactgaaaatcatcacaatgggggtctcctagcattgcatctattaatgtgcaatttcttccatctagaaccctatatgagttgttgcatattttccctattgaaatgctctggactaattcagtagcatttgtcacctcaatttggtcatcagttattgtttttaccaatgttccatttgctactgcatggtgtcccagacataatgtggctgtgttgttgccactggttgggttttgactgtagacccaatgggtcagtagtatcaaaataatggttgtcttcattacgatacaaacttaacggatatcgcgataatgaaataatttatgattatttctcgctttcaatttaacacaaccctcaagaacctttgtatttattttcactttttaagtatagaataaagaagctctaattaattaacgagcagatagtctcgttctcgccctgcctgatgactaattaattaacccggatcctttttatagctaattagtcacgtacctttgagagtaccacttcagctacctcttttgtgtctcagagtaactttctttaatcaattccaaaacagtatatgattttccatttctttcaaagatgtagtttacatctgctcctttgttgaaaagtagcctgagcacttcttttctaccatgaattacagctggcaagatcaatttttcccagttctggacattttattttttttaagtagtgtgctacatatttcaatatttccagattgtacagcgatcattaaaggagtacgtcccatgttatccagcaagtcagtatcagcacctttgttcaatagaagtttaaccattgttaaatttttatttgatacggctatatgtagaggagttaaccgatccgtgtttgaaatatctacatccgccgaatgagccaatagaagtttaaccaaattaactttgttaaggtaagctgccaaacacaaaggagtaaagcctccgctgtaaagaacattgtttacatagttattcttcaacagatctttcactattttgtagtcgtctctcaacaccgcatcatgcagacaagaagttgtgcattcagtaactacaggtttagctccatacctcatcaagatttttatagcctcggtattcttgaacattacagccatttcaagaggagattgtagagtaccatattccgtgttagggtcgaatccattgtccaaaaacctatttagagatgcattgtcattatccatgatagcctcacagacgtatatgtaagccatcttgaatgtataattttgttgttttcaacaaccgctcgtgaacagcttctatactttttcattttcttcatgattaatatagtttacggaatataagtatacaaaaagtttatagtaatctcataatatctgaaacacatacataaaacatggaagaattacacgatgtcgttgagataaatggctttttattgtcatagtttacaaattcgcagtaatcttcatcttttacgaatattgcagaatctgttttatccaaccagtgatttttgtataatataactggtatcctatcttccgatagaatgctgttatttaacatttttgcacctattaagttacatctgtcaaatccatctttccaactgactttatgtaacgatgcgaaatagcatttatcactatgtcgtacccaattatcatgacaagattctcttaaatacgtaatcttattatctcttgcatattcgtaatagtaattgtaaagagtatacgataacagtatagatatacacgtgatataaatatttaaccccattcctgagtaaaataattacgatattacatttccttttattatttttatgttttagttatttgttaggttatacaaaaattatgtttatttgtgtatatttaaagcgtcgttaagaataagcttagttaacatattatcgcttaggttttgtagtatttgaatcctttctttaaatggattatttttccaatgcatatttatagcttcatccaaagtataacatttaacattca

SEQ ID NO: 2 - DNA, 3917 bp, Influenza A virus
gtattctaaactaggaatagatgaaattatgtgcaaaggagatacctttagatatggatctgatttatttggtttttcataatcataatctaacaacattttcactatactataccttcttgcacaagtcgccattagtagtatagacttatactttgtaaccatagtatactttagcgcgtcatcttcttcatctaaaacagatttacaacaataatcatcgtcgtcatcttcatcttcattaaagttttcatattcaataactttcttttctaaaacatcatctgaatcaataaacatagaacggtatagagcgttaatctccattgtaaaatatactaacgcgttgctcatgatgtacttttttttcattatttagaaattatgcattttagatctttataagcggccgtgattaactagtcataaaaacccgggatcgattctagactcgagggtacctcaaatgcaaatgttgcatctgatgttgcctttttggcaagcccacataatgaaacccaatagaacaacgcaaattaagaagcatgatatggcgaatgaaatccacagtatccaatctttgtagcctgatttcaactcaacacctttgatttgaaatcggttgtttaatgcttcatctctgtatatgtaatggtcatatgtcccatttcttattgatccaatgcatgcattatcacatttgtggtaaatcttgaaacatccacctcccatgtcttccgcgttttctcttaactggcgtctagtcttctcgaataatttattcatttctgcatctgttaagtcaattgtatgttgattttctagagccaccagcaattctgcattgtaggaccataggtctattttggtgtcttctacatacttctccaagtcctggattctcccttctacttctgagaattccttctctatttgatggaatttctcattggtcctttcaatcactctgtttaattttccattaatctggtcgatggctgcttgagtgctctttagatctgcagcttgtcctgttccttccgagttttgatatcggaatccataccacccatcaaccattccttcccagccgttttctatgaatcccgctattgctccaaagattcctctgatttgcttttctggtacattcctcatcccagtggccagctttaaagtgttttgcctgatatacttggggcattttccatatgtaactttgttcacattttgaaatggtttgtcgttggggatgcttccatttggtgtaatacattcagacacacaaatgtctatgggtgcatctgatctcattacagagcttttccctgttttcaatttaaaatatccccgcggtgcaactaagttgccattactgtttatcattaggatatctccaggttttacaatggtccagtatatgcttatcctgcctgattgacccctgacccacggtctagatccgatattagggattatcgtttgttgacttctttttgttgagactgttactcgtcctgattcttggatgtacaattctgtttgctgttggtttgagctcgggtgatgaatcccccagatgtatagtttgtcgaaatttttattgttaggcattgtcacattcaatgtggggtaagagtttccagattttgttagccaattcagtcggctaaagaaactatcggctgatccccttttgcaggctccacttcttccgttttgagtgacacctgtccatgtgaatccctctgctgtgaattccaatgttcctgaggatgctacaatggaccggagcgatgcatagtcagggatgtcatatgggtagcaattgctgaaagcgctgcttctttctatgaagaggtcccaattctcatactgaaagacatcacagtgggggtctcctagcattgcatctattaatgtgcaatttcttccatctagaactctatatgagttgttgcatattttccctattgaaatgctctgaactaattcagtagcatttgtcacctcaatttggtcatcagttattgtttttaccaatgttccatttgctactgcatggtgtcccagacataatgtggctgtgttgttgccactggttgggttttgactgtagacccaatgggtcagtagtatcaaaataatggttgtcttcattacgatacaaacttaacggatatcgcgataatgaaataatttatgattatttctcgctttcaatttaacacaaccctcaagaacctttgtatttattttcactttttaagtatagaataaagaagctctaattaattaacgagcagatagtctcgttctcgccctgcctgatgactaattaattaacccggatcctttttatagctaattagtcacgtacctttgagagtaccacttcagctacctcttttgtgtctcagagtaactttctttaatcaattccaaaacagtatatgattttccatttctttcaaagatgtagtttacatctgctcctttgttgaaaagtagcctgagcacttcttttctaccatgaattacagctggcaagatcaatttttcccagttctggacattttattttttttaagtagtgtgctacatatttcaatatttccagattgtacagcgatcattaaaggagtacgtcccatgttatccagcaagtcagtatcagcacctttgttcaatagaagtttaaccattgttaaatttttatttgatacggctatatgtagaggagttaaccgatccgtgtttgaaatatctacatccgccgaatgagccaatagaagtttaaccaaattaactttgttaaggtaagctgccaaacacaaaggagtaaagcctccgctgtaaagaacattgtttacatagttattcttcaacagatctttcactattttgtagtcgtctctcaacaccgcatcatgcagacaagaagttgtgcattcagtaactacaggtttagctccatacctcatcaagatttttatagcctcggtattcttgaacattacagccatttcaagaggagattgtagagtaccatattccgtgttagggtcgaatccattgtccaaaaacctatttagagatgcattgtcattatccatgatagcctcacagacgtatatgtaagccatcttgaatgtataattttgttgttttcaacaaccgctcgtgaacagcttctatactttttcattttcttcatgattaatatagtttacggaatataagtatacaaaaagtttatagtaatctcataatatctgaaacacatacataaaacatggaagaattacacgatgtcgttgagataaatggctttttattgtcatagtttacaaattcgcagtaatcttcatcttttacgaatattgcagaatctgttttatccaaccagtgatttttgtataatataactggtatcctatcttccgatagaatgctgttatttaacatttttgcacctattaagttacatctgtcaaatccatctttccaactgactttatgtaacgatgcgaaatagcatttatcactatgtcgtacccaattatcatgacaagattctcttaaatacgtaatcttattatctcttgcatattcgtaatagtaattgtaaagagtatacgataacagtatagatatacacgtgatataaatatttaaccccattcctgagtaaaataattacgatattacatttccttttattatttttatgttttagttatttgttaggttatacaaaaattatgtttatttgtgtatatttaaagcgtcgttaagaataagcttagttaacatattatcgcttaggttttgtagtatttgaatcctttctttaaatggattatttttccaatgcatatttatagcttcatccaaagtataacatttaacattca

SEQ ID NO: 3 - protein, 565 aa, Influenza A virus
MKTTIILILLTHWVYSQNPTSGNNTATLCLGHHAVANGTLVKTITDDQIEVTNATELVQSISIGKICNNSYRVLDGRNCTLIDAMLGDPHCDDFQYENWDLFIERSSAFSNCYPYDIPDYASLRSIVASSGTLEFTAEGFTWTGVTQNGRSGACKRGSADSFFSRLNWLTKSGNSYPILNVTMPNNKNFDKLYIWGIHHPSSNKEQTKLYIQESGRVTVSTERSQQTVIPNIGSRPWVRGQSGRISIYWTIVKPGDILMINSNGNLVAPRGYFKLRTGKSSVMRSDALIDTCVSECITPNGSIPNDKPFQNVNKITYGKCPKYIRQNTLKLATGMRNVPEKQIRGIFGAIAGFIENGWEGMVDGWYGFRYQNSEGTGQAADLKSTQAAIDQINGKLNRVIERTNEKFHQIEKEFSEVEGRIQDLEKYVEDTKIDLWSYNAELLVALENQHTIDLTDAEMNKLFEKTRRQLRENAEDMGGGCFKIYHKCDNACIGSIRNGTYDHYIYRDEALNNRFQIKSVELKSGYKDWILWISFAISCFLICVVLLGFIMWACQKGNIRCNICI

SEQ ID NO: 4 - protein, 565 aa, Influenza A virus
MKTTIILILLTHWAYSQNPISGNNTATLCLGHHAVANGTLVKTISDDQIEVTNATELVQSISMGKICNNSYRILDGRNCTLIDAMLGDPHCDAFQYENWDLFIERSSAFSNCYPYDIPDYASLRSIVASSGTLEFTAEGFTWTGVTQNGRSGACKRGSADSFFSRLNWLTKSGSSYPTLNVTMPNNKNFDKLYIWGIHHPSSNQEQTKLYIQESGRVTVSTKRSQQTIIPNIGSRPWVRGQSGRISIYWTIVKPGDILMINSNGNLVAPRGYFKLKTGKSSVMRSDVPIDICVSECITPNGSISNDKPFQNVNKVTYGKCPKYIRQNTLKLATGMRNVPEKQIRGIFGAIAGFIENGWEGMVDGWYGFRYQNSEGTGQAADLKSTQAAIDQINGKLNRVIERTNEKFHQIEKEFSEVEGRIQDLEKYVEDTKIDLWSYNAELLVALENQHTIDLTDAEMNKLFEKTRRQLKENAEDMGGGCFKIYHKCDNACIGSIRNGTYDHYIYRDEALNNRFQIKGVELKSGYKDWILWISFAISCFLICVVLLGFIMWACQKGNIRCNICI

SEQ ID NO: 5 - protein, 565 aa, Influenza A virus; identical to SEQ ID NO: 4

SEQ ID NO: 6 - DNA, 3973 bp, Influenza A virus
  CDS (1800)..(3494)
ggaaacagctatgaccatgattacgaattgcggccgcaattctgaatgttaaatgttatactttggatgaagctataaatatgcattggaaaaataatccatttaaagaaaggattcaaatactacaaaacctaagcgataatatgttaactaagcttattcttaacgacgctttaaatatacacaaataaacataatttttgtataacctaacaaataactaaaacataaaaataataaaaggaaatgtaatatcgtaattattttactcaggaatggggttaaatatttatatcacgtgtatatctatactgttatcgtatactctttacaattactattacgaatatgcaagagataataagattacgtatttaagagaatcttgtcatgataattgggtacgacatagtgataaatgctatttcgcatcgttacataaagtcagttggaaagatggatttgacagatgtaacttaataggtgcaaaaatgttaaataacagcattctatcggaagataggataccagttatattatacaaaaatcactggttggataaaacagattctgcaatattcgtaaaagatgaagattactgcgaatttgtaaactatgacaataaaaagccatttatctcaacgacatcgtgtaattcttccatgttttatgtatgtgtttcagatattatgagattactataaactttttgtatacttatattccgtaaactatattaatcatgaagaaaatgaaaaagtatagaagctgttcacgagcggttgttgaaaacaacaaaattatacattcaagatggcttacatatacgtctgtgaggctatcatggataatgacaatgcatctctaaataggtttttggacaatggattcgaccctaacacggaatatggtactctacaatctcctcttgaaatggctgtaatgttcaagaataccgaggctataaaaatcttgatgaggtatggagctaaacctgtagttactgaatgcacaacttcttgtctgcatgatgcggtgttgagagacgactacaaaatagtgaaagatctgttgaagaataactatgtaaacaatgttctttacagcggaggctttactcctttgtgtttggcagcttaccttaacaaagttaatttggttaaacttctattggctcattcggcggatgtagatatttcaaacacggatcggttaactcctctacatatagccgtatcaaataaaaatttaacaatggttaaacttctattgaacaaaggtgctgatactgacttgctggataacatgggacgtactcctttaatgatcgctgtacaatctggaaatattgaaatatgtagcacactacttaaaaaaaataaaatgtccagaactgggaaaaattgatcttgccagctgtaattcatggtagaaaagaagtgctcaggctacttttcaacaaaggagcagatgtaaactacatctttgaaagaaatggaaaatcatatactgttttggaattgattaaagaaagttactctgagacacaaaagaggtagctgaagtggtactctcaaaggtacgtgactaattagctataaaaaggatccgggttaattaattagtcatcaggcagggcgagaacgagactatctgctcgttaattaattagagcttctttattctatacttaaaaagtgaaaataaatacaaaggttcttgagggttgtgttaaattgaaagcgagaaataatcataaattatttcattatcgcgatatccgttaagtttgtatcgtaatgaaaaccaccatcatcctgatcctgctgacccactgggcctacagccagaaccctatcagcggcaacaacaccgccaccctgtgcctgggccaccacgccgtggccaacggcaccctggtcaagaccatcagcgacgaccagatcgaagtgaccaacgccaccgagctggtgcagagcatcagcatgggcaagatctgcaacaacagctaccgcatcctggacggcagaaactgcaccctgatcgacgccatgctgggcgacccccactgcgacgccttccagtacgagaactgggacctgttcatcgagaggagcagcgccttcagcaactgctacccctacgacatccctgactacgccagcctgagaagcatcgtggccagcagcggcaccctggagttcaccgccgagggcttcacctggaccggcgtgacccagaacggcagaagcggcgcctgcaagagaggcagcgccgacagcttcttcagccgcctgaactggctgaccaagagcggcagcagctaccccaccctgaacgtgaccatgcccaacaacaagaacttcgacaagctgtacatctggggcatccaccaccccagcagcaaccaggagcagaccaagctgtacatccaggagagcggcagagtgaccgtgtccaccaagagaagccagcagaccatcatccccaacatcggcagcagaccttgggtgcgcggccagtccggcaggatcagcatctactggaccatcgtgaagcctggcgacatcctgatgatcaacagcaacggcaacctggtggcccccagaggctacttcaagctgaaaaccggcaagagcagcgtgatgagaagcgacgtgcccatcgacatctgcgtgtccgagtgcatcacccctaacggcagcatcagcaacgacaagcccttccagaacgtgaacaaagtgacctacggcaagtgccccaagtacatccgccagaacaccctgaagctggccaccggcatgagaaacgtgcccgagaagcagatcagaggcatcttcggcgccatcgccggcttcatcgagaacggctgggagggcatggtggacggctggtacggcttcagataccagaacagcgagggcaccggccaggccgccgacctgaagagcacccaggccgccatcgaccagatcaacggcaagctgaaccgcgtgatcgagcgcaccaacgagaagttccaccagatcgagaaggagttcagcgaagtggagggcagaatccaggacctggagaagtacgtggaggacaccaagatcgacctgtggagctacaacgccgagctgctggtcgccctggagaaccagcacaccatcgacctgaccgacgccgagatgaacaagctgttcgaaaagaccaggcgccagctgaaggaaaacgccgaggacatgggcggcggctgcttcaagatctaccacaagtgcgacaacgcctgcatcggctccatcaggaacggcacctacgaccactacatctacagggacgaggccctgaacaaccgcttccagatcaagggcgtggagctgaagagcggctacaaggactggatcctgtggatcagcttcgccatcagctgcttcctgatctgcgtggtgctgctgggcttcatcatgtgggcctgccagaagggcaacatccgctgcaacatctgcatctgatgactcgagggtttttatgactagttaatcacggccgcttataaagatctaaaatgcataatttctaaataatgaaaaaaagtacatcatgagcaacgcgttagtatattttacaatggagattaacgctctataccgttctatgtttattgattcagatgatgttttagaaaagaaagttattgaatatgaaaactttaatgaagatgaagatgacgacgatgattattgttgtaaatctgttttagatgaagaagatgacgcgctaaagtatactatggttacaaagtataagtctatactactaatggcgacttgtgcaagaaggtatagtatagtgaaaatgttgttagattatgattatgaaaaaccaaataaatcagatccatatctaaaggtatctcctttgcacataatttcatctattcctagtttagaatacctgcagccaagcttggcactggccgtcgttttac

SEQ ID NO: 7 - DNA, 3973 bp, Influenza A virus
gtaaaacgacggccagtgccaagcttggctgcaggtattctaaactaggaatagatgaaattatgtgcaaaggagatacctttagatatggatctgatttatttggtttttcataatcataatctaacaacattttcactatactataccttcttgcacaagtcgccattagtagtatagacttatactttgtaaccatagtatactttagcgcgtcatcttcttcatctaaaacagatttacaacaataatcatcgtcgtcatcttcatcttcattaaagttttcatattcaataactttcttttctaaaacatcatctgaatcaataaacatagaacggtatagagcgttaatctccattgtaaaatatactaacgcgttgctcatgatgtactttttttcattatttagaaattatgcattttagatctttataagcggccgtgattaactagtcataaaaaccctcgagtcatcagatgcagatgttgcagcggatgttgcccttctggcaggcccacatgatgaagcccagcagcaccacgcagatcaggaagcagctgatggcgaagctgatccacaggatccagtccttgtagccgctcttcagctccacgcccttgatctggaagcggttgttcagggcctcgtccctgtagatgtagtggtcgtaggtgccgttcctgatggagccgatgcaggcgttgtcgcacttgtggtagatcttgaagcagccgccgcccatgtcctcggcgttttccttcagctggcgcctggtcttttcgaacagcttgttcatctcggcgtcggtcaggtcgatggtgtgctggttctccagggcgaccagcagctcggcgttgtagctccacaggtcgatcttggtgtcctccacgtacttctccaggtcctggattctgccctccacttcgctgaactccttctcgatctggtggaacttctcgttggtgcgctcgatcacgcggttcagcttgccgttgatctggtcgatggcggcctgggtgctcttcaggtcggcggcctggccggtgccctcgctgttctggtatctgaagccgtaccagccgtccaccatgccctcccagccgttctcgatgaagccggcgatggcgccgaagatgcctctgatctgcttctcgggcacgtttctcatgccggtggccagcttcagggtgttctggcggatgtacttggggcacttgccgtaggtcactttgttcacgttctggaagggcttgtcgttgctgatgctgccgttaggggtgatgcactcggacacgcagatgtcgatgggcacgtcgcttctcatcacgctgctcttgccggttttcagcttgaagtagcctctgggggccaccaggttgccgttgctgttgatcatcaggatgtcgccaggcttcacgatggtccagtagatgctgatcctgccggactggccgcgcacccaaggtctgctgccgatgttggggatgatggtctgctggcttctcttggtggacacggtcactctgccgctctcctggatgtacagcttggtctgctcctggttgctgctggggtggtggatgccccagatgtacagcttgtcgaagttcttgttgttgggcatggtcacgttcagggtggggtagctgctgccgctcttggtcagccagttcaggcggctgaagaagctgtcggcgctgcctctcttgcaggcgccgcttctgccgttctgggtcacgccggtccaggtgaagccctcggcggtgaactccagggtgccgctgctggccacgatgcttctcaggctggcgtagtcagggatgtcgtaggggtagcagttgctgaaggcgctgctcctctcgatgaacaggtcccagttctcgtactggaaggcgtcgcagtgggggtcgcccagcatggcgtcgatcagggtgcagtttctgccgtccaggatgcggtagctgttgttgcagatcttgcccatgctgatgctctgcaccagctcggtggcgttggtcacttcgatctggtcgtcgctgatggtcttgaccagggtgccgttggccacggcgtggtggcccaggcacagggtggcggtgttgttgccgctgatagggttctggctgtaggcccagtgggtcagcaggatcaggatgatggtggttttcattacgatacaaacttaacggatatcgcgataatgaaataatttatgattatttctcgctttcaatttaacacaaccctcaagaacctttgtatttattttcactttttaagtatagaataaagaagctctaattaattaacgagcagatagtctcgttctcgccctgcctgatgactaattaattaacccggatcctttttatagctaattagtcacgtacctttgagagtaccacttcagctacctcttttgtgtctcagagtaactttctttaatcaattccaaaacagtatatgattttccatttctttcaaagatgtagtttacatctgctcctttgttgaaaagtagcctgagcacttcttttctaccatgaattacagctggcaagatcaatttttcccagttctggacattttattttttttaagtagtgtgctacatatttcaatatttccagattgtacagcgatcattaaaggagtacgtcccatgttatccagcaagtcagtatcagcacctttgttcaatagaagtttaaccattgttaaatttttatttgatacggctatatgtagaggagttaaccgatccgtgtttgaaatatctacatccgccgaatgagccaatagaagtttaaccaaattaactttgttaaggtaagctgccaaacacaaaggagtaaagcctccgctgtaaagaacattgtttacatagttattcttcaacagatctttcactattttgtagtcgtctctcaacaccgcatcatgcagacaagaagttgtgcattcagtaactacaggtttagctccatacctcatcaagatttttatagcctcggtattcttgaacattacagccatttcaagaggagattgtagagtaccatattccgtgttagggtcgaatccattgtccaaaaacctatttagagatgcattgtcattatccatgatagcctcacagacgtatatgtaagccatcttgaatgtataattttgttgttttcaacaaccgctcgtgaacagcttctatactttttcattttcttcatgattaatatagtttacggaatataagtatacaaaaagtttatagtaatctcataatatctgaaacacatacataaaacatggaagaattacacgatgtcgttgagataaatggctttttattgtcatagtttacaaattcgcagtaatcttcatcttttacgaatattgcagaatctgttttatccaaccagtgatttttgtataatataactggtatcctatcttccgatagaatgctgttatttaacatttttgcacctattaagttacatctgtcaaatccatctttccaactgactttatgtaacgatgcgaaatagcatttatcactatgtcgtacccaattatcatgacaagattctcttaaatacgtaatcttattatctcttgcatattcgtaatagtaattgtaaagagtatacgataacagtatagatatacacgtgatataaatatttaaccccattcctgagtaaaataattacgatattacatttccttttattatttttatgttttagttatttgttaggttatacaaaaattatgtttatttgtgtatatttaaagcgtcgttaagaataagcttagttaacatattatcgcttaggttttgtagtatttgaatcctttctttaaatggattatttttccaatgcatatttatagcttcatccaaagtataacatttaacattcagaattgcggccgcaattcgtaatcatggtcatagctgtttcc

SEQ ID NO: 8 - DNA, 890 bp, Influenza A virus
agcaaaagcagggtgacaaaaacataatggattccaacactgtgtcaagctttcaggtagactgttttctttggcatgtccgcaaacgattcgcagaccaagaactgggtgatgccccattccttgaccggcttcgccgagaccagaagtccctaaggggaagaggtatcactcttggtctggacatcgaaacagccactcatgcaggaaagcagatagtggagcagattctggaaaaggaatcagatgaggcacttaaaatgaccattgcctctgttcctacttcacgctacttaactgacatgactcttgatgagatgtcaagagactggttcatgctcatgcccaagcaaaaagtaacaggctccctatgtataagaatggaccaggcaatcatggataagaacatcatacttaaagcaaactttagtgtgattttcgaaaggctggaaacactaatactacttagagccttcaccgaagaaggagcagtcgttggcgaaatttcaccattaccttctcttccaggacatactaatgaggatgtcaaaaatgcaattggggtcctcatcggaggacttaaatggaatgataatacggttagaatctctgaaactctacagagattcgcttggagaagcagtcatgagaatgggagaccttcattcccttcaaagcagaaatgaaaaatggagagaacaattaagccagaaatttgaagaaataagatggttgattgaagaagtgcgacatagattgaaaaatacagaaaatagttttgaacaaataacatttatgcaagccttacaactattgcttgaagtagaacaagagataagaactttctcgtttcagcttatttaatgataaaaaacacccttgtttctact

SEQ ID NO: 9 - DNA, 1027 bp, Influenza A virus
agcaaaagcaggtagatatttaaagatgagtcttctgaccgaggtcgaaacgtacgttctctctatcgtaccatcaggccccctcaaagccgagatcgcgcagagacttgaagatgtctttgcagggaagaacaccgatcttgaggcactcatggaatggctaaagacaagaccaatcctgtcacctttgactaaagggattttaggatttgtattcacgctcaccgtgcccagtgagcgaggactgcagcgtagacgctttgtccaaaatgcccttagtggaaacggagatccaaacaacatggacagagcagtaaaactgtacaggaagcttaaaagagaaataacattccatggggcaaaagaggtggcactaagctattccactggtgcactagccagctgcatgggactcatatacaacagaatgggaactgtgacaaccgaagtggcatttggcctggtatgcgccacatgtgaacagattgctgattcccagcatcgatctcacaggcagatggtgacaacaaccaacccattaatcagacatgaaaacagaatggtattagccagtaccacggctaaagccatggaacagatggcaggatcaagtgagcaggcagcagaggccatggaggttgctagtaaggctaggcagatggtacaggcaatgagaaccattgggacccaccctagctccagtgccggtttgaaagatgatctccttgaaaatttacaggcctaccagaaacggatgggagtgcaaatgcagcgattcaagtgatcctctcgttattgcagcaagtatcattgggatcttgcacttgatattgtggattcttgatcgtcttttcttcaaattcatttatcgtcgccttaaatacgggttgaaaagagggccttctacggaaggagtacctgagtctatgagggaagaatatcggcaggaacagcagaatgctgtggatgttgacgatggtcattttgtcaacatagagctggagtaaaaaactaccttgtttctact

SEQ ID NO: 10 - DNA, 1460 bp, Influenza A virus
agcaaaagcaggagtttaaaatgaatccaaatcaaaagataatagcaattggatttgcatcattggggatattaatcattaacgtcattctccatgtagtcagcattatagtaacagtactggtcctcaataacaatggaacaggtctgaactgcaaagggacgatcataagagagtacaatgaaacagtaagagtagaaaaaattactcaatggtataataccagtgcaattaagtacatagagagacctccaaatgaatactacatgaacaacaccgaaccactttgtgaggcccaaggctttgcaccattttccaaagataatggaatacgaattgggtcgagaggccatgtttttgtgataagagaaccttttgtatcatgttcgccctcagaatgtagaacctttttcctcacacagggctcattactcaatgacaaacattctaacggcacagtaaaggaccgaagtccatataggactttgatgagtgtcaaaatagggcaatcacctaatgtgtatcaagctaggtttgaatcggtggcatggtcagcaacagcatgccatgatggaaaaaaatggatgacagttggagtcacagggcccgacaatcaagcaattgcagtagtgaactatggaggtgttccggttgatattattaattcatgggcaggggatatcttaagaacccaagaatcatcatgcacctgcattaaaggagactgttattgggtaatgactgatggaccggcaaataggcaagctaaatataggatattcaaagcaaaagatggaagagtaattggacagactgatataagtttcaatgggggacacatagaggagtgttcttgttaccccaatgaagggaaggtggaatgcatatgcagggacaattggactggaacaaatagaccaattctggtaatatcttctgatctatcgtacacagttggatatttgtgtgctggcattcccactgacactcctaggggagaggatagtcaattcacaggctcatgtacaagacctttgggaaataaaggatacggtgtaaaaggtttcgggtttcgacaaggaactgacgtatgggccggaaggacaattagtaggacttcaagatcaggattcgaaataataaaaatcaggaatggttggacacagaacagtaaagaccaaatcaggaggcaagtgattatcgatgacccaaattggtcaggatatagcggttctttcacattgccggttgaactaacaaaaaagggatgtttggtcccctgtttctgggttgaaatgattagaggtaaacctgaagaaacaacaatatggacctctagcagctccattgtgatgtgtggagtagatcataaaattgccagttggtcatggcacgatggagctattcttccctttgacatcgataagatgtaatttacgaaaaaactccttgtttctact

SEQ ID NO: 11 - DNA, 1565 bp, Influenza A virus
agcaaaagcagggtagataatcactcactgagtgacatcaaagtcatggcgtctcaaggcaccaaacgatcctatgaacagatggaaactgatggggaacgccagaatgcaactgaaatcagagcatctgtcggaaggatggtgggaggaatcggccggttttatgttcagatgtgtactgagcttaaactaaacgaccatgaagggcggctgattcagaacagcataacaatagaaaggatggtactttcggcattcgacgaaagaagaaacaagtatctcgaggagcatcccagtgctggaaaagaccctaagaaaacgggaggcccgatatacagaaggaaagatgggaaatggatgagggaactcatcctccatgataaagaagaaatcatgagaatctggcgtcaggccaacaatggtgaagacgctactgctggtcttactcatatgatgatctggcactccaatctcaatgacaccacataccaaagaacaagggctcttgttcggactgggatggatcccagaatgtgctctctgatgcaaggctcaaccctcccacggagatctggagccgctggtgctgcagtaaaaggtgttggaacaatggtaatggaactcatcagaatgatcaaacgcggaataaatgatcggaatttctggagaggtgaaaatggtcgaaggaccagaattgcttatgaaagaatgtgcaatatcctcaaagggaaatttcagacagcagcacaacgggctatgatggaccaggtgagggaaggccgcaatcctggaaacgctgagattgaggatctcattttcttagcacgatcagcacttattttgagaggatcagtagcccataaatcatgcctacctgcctgtgtttatggccttgcagtaaccagtgggtatgactttgagaaggaaggatactctctggttggaattgatcctttcaaactactccagaacagtcaaattttcagtctaatcagaccaaaagaaaacccagcacacaagagccagttggtgtggatggcatgccattctgcagcatttgaggacctgagagttttgaatttcattagaggaaccaaagtaatcccaagaggacagttaacaaccagaggagttcaaattgcttcaaatgaaaacatggagacaatagattctagcacacttgaactgagaagcaaatattgggcaataaggaccagaagtggaggaaacaccagtcaacagagagcatctgcaggacagataagtgtgcaacctactttctcagtacagagaaatcttccctttgagagagcaaccattatggctgcattcactggtaacactgaagggaggacttccgacatgagaacggaaatcataaggatgatggaaagtgccaaatcagaagatgtgtctttccaggggcggggagtcttcgagctctcggacgaaaaggcaacgaacccgatcgtgccttcctttgacatgagcaatgaagggtcttatttcttcggagacaatgctgaggaatttgacagttaaagaaaaatacccttgtttctact

SEQ ID NO: 12 - DNA, 1762 bp, Influenza A virus
agcaaaagcaggggatatttctgtcaatcatgaagacaaccattattttgatactactgacccattgggcctacagtcaaaacccaatcagtggcaacaacacagccacattgtgtctgggacaccatgcagtagcaaatggaacattggtaaaaacaataagtgatgatcaaattgaggtgacaaatgctacagaattagttcagagcatttcaatggggaaaatatgcaacaactcatatagaattctagatggaagaaattgcacattaatagatgcaatgctaggagacccccactgtgacgtctttcagtatgagaattgggacctctttatagaaagaagcagcgctttcagcaattgctacccatatgacatccctgactatgcatcgctccgatccattgtagcatcctcaggaacattggaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaagctatacatctgggggattcatcacccgagctcaaatcaagagcagacaaaattgtacatccaagaatcaggacgagtaacagtctcaacaaaaagaagtcaacaaacaataatccctaacatcggatctagaccgtgggtcagaggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatctccaacgacaagccattccaaaatgtgaacaaagttacatatggaaaatgccccaagtatatcaggcaaaacactttaaagttggccactgggatgaggaatgtaccagaaaagcaaatcagaggaatctttggagcaatagcgggattcatcgaaaacggctgggaaggaatggttgatgggtggtatgggttccgatatcaaaactctgaaggaacagggcaagctgcagatctaaagagcactcaagcagccatcgaccagattaatggaaagttaaacagagtgattgaaagaaccaatgagaaattccatcaaatagagaaggaattctcagaagtagaaggaagaattcaggacttggagaaatatgtagaagacaccaaaatagacctatggtcctacaatgcagaattgctggtggctctagaaaatcaacatacaattgacttaacagatgcagaaatgaataaattattcgagaagactagacgccagttaagagaaaacgcagaagacatgggaggtggatgtttcaagatttaccacaaatgtgataatgcatgcattggatcaataagaaatgggacatatgaccattacatatacagagatgaagcattaaacaaccgatttcaaatcaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 13 - DNA, 2233 bp, Influenza A virus
agcgaaagcaggtactgatccaaaatggaagactttgtgcgacagtgcttcaatccaatgatcgtcgagcttgcggaaaaggcaatgaaagaatatggagaggacccgaaaatcgaaacaaacaaatttgcagcaatatgcactcacttggaagtctgcttcatgtactcggatttccactttattaatgaactgggtgagtcagtggtcatagagtctggtgacccaaatgctcttttgaaacacagatttgaaatcattgaggggagagatcgaacaatggcatggacagtggtaaacagcatctgcaacaccacaagagctgaaaaacctaaatttcttccagatttatacgactataaggagaacagatttgttgaaattggtgtgacaaggagagaagttcacatatactacctggagaaggccaacaaaataaagtctgagaaaacacatatccacattttctcatttacaggagaggaaatggctacaaaagcggactatactcttgatgaagagagtagagccaggatcaagaccagactattcactataagacaagaaatggccagtagaggcctctgggattcctttcgtcagtccgagagaggcgaagagacaattgaagaaagatttgaaatcacagggacgatgcgcaagcttgccaattacagtctcccaccgaacttctccagccttgaaaattttagagtctatgtggatggattcgaaccgaacggctgcattgagagtaagctttctcaaatgtccaaagaagtaaatgccagaatcgaaccattttcaaagacaacaccccgaccactcaaaatgccaggtggtccaccctgccatcagcgatctaaattcttgctaatggatgctctgaaactgagcattgaggacccaagtcacgagggagagggaataccactatatgatgcaatcaaatgcatgaaaactttctttggatggaaagagcccagtattgttaaaccacatgaaaagggtataaacccgaactatctccaaacttggaagcaagtattagaagaaatacaagaccttgagaacgaagaaaggacccccaagaccaagaatatgaaaaaaacaagccaattgaaatgggcactaggtgaaaatatggcaccagagaaagtggattttgaggattgtaaagacatcagtgatttaaaacagtatgacagcgatgagccagaaacaaggtctcttgcaagttggattcaaagtgagttcaacaaagcttgtgagctgacagattcaagctggatagagctcgatgaaattggggaggatgtcgccccaatagaatacattgcgagcatgaggagaaattattttactgctgagatttcccattgtagagcaacagaatatataatgaaaggagtgtacatcaacactgctctactcaatgcatcctgtgctgcgatggatgaatttcaattaattccgatgataagtaaatgcaggaccaaagaagggagaaggaaaacaaatttatatggattcataataaagggaaggtcccatttaagaaatgatactgacgtggtgaactttgtaagtatggaattttctctcactgatccaagatttgagccacacaaatgggaaaaatactgcgttctagaaattggagacatgcttctaaggactgctgtaggtcaagtgtcaagacccatgtttttgtatgtaaggacaaatggaacctctaaaattaaaatgaaatggggaatggaaatgaggcgctgcctccttcagtctctgcaacagattgaaagcatgatcgaagctgagtcctcagtcaaagaaaaggacatgaccaaagaattttttgagaacaaatcagagacatggcctataggagagtcccccaaaggagtggaagagggctcaatcgggaaggtttgcaggaccttattagcaaaatctgtgtttaacagtttatatgcatctccacaactggaagggttttcagctgaatctaggaaattacttctcattgttcaggctcttagggataacctggaacctggaacctttgatattggggggttatatgaatcaattgaggagtgcctgattaatgatccctgggttttgcttaatgcatcttggttcaactccttccttacacatgcactgaagtagttgtggcaatgctactatttgctatccatactgtccaaaaaagtaccttgtttctact

SEQ ID NO: 14 - DNA, 2341 bp, Influenza A virus
agcgaaagcaggcaaaccatttgaatggatgtcaatccgactctacttttcttaaaggtgccagcgcaaaatgctataagcacaacattcccttatactggagatcctccctacagtcatggaacagggacaggatacaccatggatactgtcaacagaacacaccaatattcagaaaaggggaaatggacaacaaacactgagattggagcaccacaacttaatccaatcgatggaccacttcctgaagacaatgaaccaagtgggtacgcccaaacagattgtgtattggaagcaatggctttccttgaagaatcccatcccggaatctttgaaaattcgtgtcttgaaacgatggaggtgattcagcagacaagagtggacaaactaacacaaggccgacaaacttatgattggaccttgaataggaatcaacctgccgcaacagcacttgctaatacaattgaagtgttcagatcaaatggtctgacttccaatgaatcagggaggttgatggacttcctcaaagatgtcatggagtccatgaacaaggaagaaatggaaataacaacacacttccaacgaaagagaagagtaagagacaacatgacaaagagaatggtaacacagagaaccatagggaagaaaaaacaacgattaaacagaaagagttatctaatcagaacattaaccctaaacacaatgaccaaggacgctgagagagggaaattgaaacgacgagcaatcgcaaccccagggatgcagataagaggatttgtatattttgttgaaacactagcccgaagaatatgtgaaaagcttgaacaatcaggattgccagttggcggtaatgagaaaaaggccaaactggctaatgtcgtcagaaaaatgatgactaattcccaagacactgaactctccttcaccatcactggggacaataccaaatggaatgaaaatcagaacccacgcatattcctggcaatgatcacatacataactagaaaccagccagaatggttcagaaatgttctaagcattgcaccgattatgttctcaaataaaatggcaagactggggaaaggatatatgtttgaaagcaaaagtatgaaactgagagctcaaataccagcagaaatgctagcaagcattgacctgaaatatttcaatgattcaacaaaaaagaaaattaaaaagatacgaccacttctggttgacgggactgcttcactgagtcctggcatgatgatgggaatgttcaacatgttgagcactgtgctgggtgtatccatattaaacctgggccagaggaaatacacaaagaccacatactggtgggatggtctgcaatcatccgatgactttgctttgatagtgaatgcgcctaatcatgaaggaatacaagctggagtagacagattctatagaacttgcaaactggtcgggatcaacatgagcaaaaagaagtcctacataaatagaaccggaacattcgaattcacaagctttttctaccggtatggttttgtagccaatttcagcatggaactacccagttttggggtttccggaataaatgaatctgcagacatgagcattggagtgacagtcatcaaaaacaacatgataaataatgatctcggtcctgccacggcacaaatggcactccaactcttcattaaggattaccggtacacataccggtgccatagaggtgatacccagatacaaaccagaagatcttttgagttgaagaaactgtgggaacagactcgatcaaagactggtctactggtatcagatgggggtccaaacctatataacatcagaaacctacacatcccggaagtttgtttaaaatgggagctaatggatgaagattataaggggaggctatgcaatccattaaatcctttcgttagtcacaaagaaattgaatcagtcaacagtgcagtagtaatgcctgcgcatggccctgccaaaagcatggagtatgatgctgttgcaacaacacattcttggatccccaagaggaaccggtccatattgaacacaagccaaaggggaatactcgaagatgagcagatgtatcagaaatgctgcaacctgtttgaaaaattcttccccagcagctcatacagaagaccagtcggaatttctagtatggttgaggccatggtgtccagggcccgcattgatgcacgaattgacttcgaatctggacggataaagaaggatgagttcgctgagatcatgaagatctgttccaccattgaagagctcagacggcaaaaatagtgaatttagcttgatcttcatgaaaaaatgccttgtttctact

SEQ ID NO: 15 - DNA, 2341 bp, Influenza A virus
agcgaaagcaggtcaaatatattcaatatggagagaataaaagaactgagagatctgatgttacaatcccgcacccgcgagatactaacaaaaactactgtggaccacatggccataatcaagaaatacacatcaggaagacaagagaagaaccctgcacttaggatgaaatggatgatggcaatgaaatacccaattacagcagataagaggataatggagatgattcctgagagaaatgaacagggacaaaccctttggagcaaaacgaacgatgctggctcagaccgcgtaatggtatcacctctggcagtgacatggtggaataggaatggaccaacaacgagcacaattcattatccaaaagtctacaaaacttattttgaaaaggttgaaagattgaaacacggaacctttggccccgttcattttaggaatcaagtcaagataagacgaagagttgatgtaaaccctggtcacgcggacctcagtgccaaagaagcacaagatgtgatcatggaagttgttttcccaaatgaagtgggagccagaattctaacatcggaatcacaactaacaataaccaaagagaaaaaggaagaacttcaggactgcaaaattgctcccttgatggtagcatacatgctagaaagagagttggtccgaaaaacaaggttcctcccagtggcaggcggaacaagcagtgtatacattgaagtgttgcatctgactcagggaacatgctgggagcaaatgtacaccccaggaggagaagttagaaacgatgatattgatcaaagtttaattattgcagcccggaacatagtgagaagagcgacagtatcagcagatccactagcatccctactggaaatgtgccacagtacacagattggtggaataaggatggtagacatccttaagcagaatccaacagaggaacaagctgtggatatatgcaaagcagcaatgggattgagaattagctcatcattcagctttggtggattcaccttcaaaagaacaagtggatcatcagtcaagagagaagaagaaatgcttacgggcaaccttcaaacattgaaaataagagtgcatgagggctatgaagaattcacaatggtcggaagaagagcaacagccattctcagaaaggcaaccagaagattgattcaattgatagtaagtgggagagatgaacaatcaattgctgaagcaataattgtagccatggtgttttcgcaagaagattgcatgataaaagcagttcgaggcgatttgaactttgttaatagagcaaatcagcgtttgaaccccatgcatcaactcttgaggcatttccaaaaagatgcaaaagtgcttttccaaaattggggaattgaacccatcgacaatgtaatgggaatgattggaatattgcctgacatgaccccaagcaccgagatgtcattgagaggagtgagagtcagcaaaatgggagtggatgagtactccagcactgagagagtggtggtgagcattgaccgttttttaagagttcgggatcaaaggggaaacatactactgtcccctgaagaagtcagtgaaacacaaggaacggaaaagctgacaataatttattcgtcatcaatgatgtgggagattaatggtcccgaatcagtgttggtcaatacttatcaatggatcatcaggaactgggaaattgtaaaaattcagtggtcacaggaccccacaatgttatacaataagatagaatttgagccattccagtccctggtccctagggccaccagaagccaatacagcggtttcgtaagaaccctgtttcagcaaatgcgagatgtacttggaacatttgatactgctcaaataataaaactcctcccttttgccgctgctcctccggaacagagtaggatgcagttctcttctttgactgttaatgtaagaggatcgggaatgaggatacttgtaagaggcaattccccagtgttcaactacaataaagccactaaaaggctcacagtcctcggaaaggatgcaggtgcgcttactgaggacccagatgaaggtacggctggagtagaatctgctgttctaagagggtttctcattttaggtaaagaaaacaagagatatggcccagcactaagcatcaatgaactaagcaaacttgcaaaaggggagaaagccaatgtactaattgggcaaggggacgtagtgttggtaatgaaacggaaacgtgactctagcatacttactgacagccagacagcgaccaaaaggattcggatggccatcaattagtgttgaattgtttaaaaacgaccttgtttctact

SEQ ID NO: 16 - DNA, 1762 bp, Influenza A virus
agcaaaagcaggggatatttctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtctttcagtatgagaattgggacctcttcatagaaagaagcagcgctttcagcagttgctacccatatgacatccctgactatgcatcgctccggtccattgtagcatcctcaggaacattggaattcacagcagagggattcacatggacaggtgtcactcaaaacggaagaagtggatcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaacgaagagcagacaaaattgtacatccaagaatcaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcagaggaatctttggagcaatagcgggattcatagaaaacggctgggaaggaatggttgatgggtggtatggattccgatatcaaaactcggaaggaacaggacaagctgcagatctaaagagcactcaagcagccatcgaccagatcaatggaaaattaaacagagtgattgaaaggaccaatgagaaattccatcaaatagagaaggaattctcagaagtagaagggagaatccaggatttggagaagtatgtagaagacaccaaaatagacctatggtcctacaatgcagaattgctggtggctctagaaaatcaacatacaattgacttaacagatgcagaaatgaataaattattcgagaagactagacgccagttaagagaaaacgcggaagacatgggaggtggatgtttcaagatataccacaaatgtgataatgcatgcattggatcaataagaaatgggacatatgaccattacatatacagagatgaagcattaaacaaccggtttcaaatcaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 17 - DNA, 1762 bp, Influenza A virus
agcaaaagcaggggatatttctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtctttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaacaatgagcagacaaaattgtatatccaagaaacaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagcaatggcaacttagttgcaccgcggggatattttaaattgagaacagggagaagctctgtaatgagatcagatgcacccatagacatttgtgtgtctgaatgtattacaccaaatggaagcatccccaacgacaaaccatttcaaaatgtgaacaaagttacatatggaaaatgccccaaatatatcaggcaaaacactttaaagctggccactgggatgaggaatgtaccagaaaagcaaatcagaggaatctttggagcaatagcgggattcatagaaaacggctgggaaggaatggttgatgggtggtatggattccgatatcaaaactcggaaggaacaggacaagctgcagatctaaagagcactcaagcagccatcgaccagatcaatggaaaattaaacagagtgattgaaaggaccaatgagaaattccatcaaatagagaaggaattctcagaagtagaagggagaatccaggatttggagaagtatgtagaagacaccaaaatagacctatggtcctacaatgcagaattgctagtggctctagaaaatcaacatacaattgacttaacagacgcagaaatgaataaattattcgagaagactagacgccagttaagagaaaacgcggaagacatgggaggtggatgtttcaagatttaccacaaatgtgataatgcatgcattggatcaataagaaatgggacatatgaccattacatatacagagatgaagcattaaacaaccgatttcaaatcaaaggtgttgaattaaaatcaggctacaaagattggatactgtggatttcattcgccatatcatgcttcttaatttgtgttgttctattgggtttcattatgtgggcttgccaaaaaggcaacatcagatgcaacatttgcatttgagtaaactgatagttaaaaacacccttgtttctact

SEQ ID NO: 18 - DNA, 1762 bp, Influenza A virus
agcaaaagcaggggatatttctgtcaatcatgaaaacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaataggaaaaatatgcaacaactcatatagggttctagatggaagaaattgcacattaatagatgcaatgctaggagaccctcactgtgatgtctttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaggaagtggagcctgcaaaagaggatcagccgatagtttctttagccgactgaattggctaacaaaatctggaaactcttaccccacattgaatgtgacaatgcctaacaataaaaatttcgacaaactatacatctgggggattcatcacccgagctcaaacaatgagcagacaaaattgtatatccaagaatcaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagtaatggcaacttagttgcaccgcggggatattttaaattgagaacagggaaaagctctgtaatgagatcagatgcacccatagacacttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcagaggaatctttggagcaatagcgggattcatagaaaacggctgggaaggaatggttgatgggtggtatggattccgatatcaaaactcggaaggaacaggacaagctgcagatctaaagagcactcaagcagccatcgaccagatcaatggaaaattaaacagagtgattgaaaggaccaatgagaaattccatcaaatagagaaggaattctcagaagtagaagggagaatcaaggacttggagaagtatgtagaagacaccaaaatagacctatggtcctacaatgcagaattgctggtggctctagaaaatcaacatacaattgacttaacagatgcagaaatgaataaattattcgagaagactagacgccagttaagagaaaacgcggaagacatgggaggtggatgtttcaagatctaccacaaatgtgataatgcatgcattggatcaataagaaatgggacatatgaccattacatatacaaagatgaagcattaaacaaccgatttcaaatcaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 19 - DNA, 1762 bp, Influenza A virus
agcaaaagcaggggatatttctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtctttcagtatgagaattgggacctcttcatagaaagaagcagcgctttcagcaattgctacccatatgacatccctgactatgcatcgctccggtccattgtagcatcctcaggaacattggaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaacagcaaacagaattgtacatccaagaatcaggacgagtaacagtctcaacaaaaagaagtcaacaaacgataatccctaatatcggatctagaccgtgggtcaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcagaggaatctttggagcaatagcgggattcatagaaaacggctgggaaggaatggttgatgggtggtatggattccgatatcaaaactcggaaggaacaggacaagctgcagatctaaagagcactcaagcagccatcgaccagattaatggaaaattaaacagagtgattgaaaggaccaatgagaaattccatcaaatagagaaggaattctcagaagtagaagggagaatccaggacttggagaagtatgtagaagacaccaaaatagacctatggtcctacaatgcagaattgctggtggctctagaaaatcaacatacaattgacttaacagatgcagaaatgaataaattattcgagaagactagacgccagttaagagaaaacgcggaagacatgggaggtggatgtttcaagatttaccacaaatgtgataatgcatgcattggatcaataagaaatgggacatatgaccattacatatacagagatgaagcattaaacaaccgatttcaaatcaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 20 - DNA, 1565 bp, Influenza A virus
agcaaaagcagggtagataatcactcactgagtgacatcaaagtcatggcgtctcaaggcaccaaacgatcttatgagcagatggaaactggtggggaacgccagaatgcaactgaaatcagagcatctgttggaaggatggtgggaggaatcggccggttctatgttcaaatgtgtactgagcttaaactcaacgaccatgaagggcggctgattcagaacagcataacaatagaaaggatggtactttcggcattcgacgaaagaagaaacaagtacctcgaggagcatcccagtgctgggaaagaccccaagaaaacgggaggcccgatatacagaaggagagatgggaaatggatgagagaactcatcctccatgataaagaagaaatcatgaggatctggcgtcaggccaacaatggtgaagacgctactgctggtcttactcatatgatgatctggcactccaatctcaatgacaccacctaccaaagaacaagggctcttgttcgggctgggatggatcccagaatgtgctctctgatgcaaggatcaactctcccacggagatctggagctgccggtgctgcagtgaagggtgttggaacaatggtaatggaactcatcaggatgatcaaacgcgggataaatgatcgaaacttctggagaggtgaaaatggtcgaagaaccagaattgcttatgaaagaatgtgcaacatcctcaaggggaaattccaaacagcagcacaacgagcaatgatggaccaagtgagggagggccgcaatcctggaaatgctgagattgaggatctcattttcttggcacgatcagcactcattctgagaggatcagtagcccataaatcatgcctacctgcctgtgtttatggccttgcagtagccagtgggtatgactttgagaaagagggatactctctggttggaattgatcctttcaaactactccagaacagccaaattttcagtctaatcagaccgaaagaaaatccagcacacaagagccagctggtgtggatggcatgccattctgcagcatttgaggacctgagagtttcgaatttcattagaggaaccaaagtaatcccaagaggacagttagcaaccaggggagtgcaaattgcttcaaatgaaaacatggagacaatagattctagcacactcgaactgaggagcagatattgggcaataaggaccaggagtggggggaacaccagtcaacagagagcatctgcaggacagataagtgtgcaacccactttctcagtgcagagaaatcttccctttgaaagagcaaccattatggctgcattcactggaaacactgaggggaggacttccgacatgagaacggaaatcataaggatgatggaaaatgccagatcagaagatgtgtctttccaggggcggggagtcttcgagctctcggacgaaaaggcaacgaacccgatcgtgccttcctttgacatgagcaatgaagggtcttatttcttcggagacaatgctgaggagtttgacagttaaagaaaaatacccttgtttctact

SEQ ID NO: 21 - DNA, 1096 bp, Influenza A virus
gtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtctttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaggaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaacaatgagcagacaaaattgtatatccaagaaacaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagcaatggcaacttagttgcaccgcggggatattttaaattgagaacagggagaagctctgtaatgagatcagatgcacccatagacatttgtgtgtctgaatgtattacaccaaatggaagcatccccaacgacaaaccatttcaaaatgtgaacaaagttacatatggaaaatgccccaaatatatcaggcaaaacactttaaagctggccactgggatgaggaatgtaccagaaaagcaaatcagaggaatctttggagcaatagcgggattcatagaaaacggctgggaaggaatggttgat

SEQ ID NO: 22 - DNA, 1061 bp, Influenza A virus
agcaaaagcaggggatatttctgtcaatcatgaagacaaccattattttgatactactgacccattgggcctacagtcaaaacccaatcagtggcaacaacacagccacattgtgtctgggacaccatgcagtagcaaatggaacattggtaaaaacaataactgatgatcaaattgaggtgacaaatgctactgaattagttcagagcatttcaatggggaaaatatgcaacaactcatatagagttctagatggaagaaattgcacattaatagatgcaatgctaggagacccccactgtgatgtctttcagtatgagaattgggacctctttatagaaagaagcagcgctttcagcaattgctacccatatgacatccctgactatgcatcgctccggtccattgtagcatcctcaggaacattggaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaagagcagacaaaattgtacatccaagaatcaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatctccaacgacaaaccattccaaaatgtgaacaaagttacatatggaaaatgccccaagtatatcaggcaaaacactttaaagctggccactgggatgaggaatgaaccagaaaagcaaatcaga

SEQ ID NO: 23 - DNA, 1061 bp, Influenza A virus
agcaaaagcaggggatatttctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtcttccagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaaaagcagacagaattgtacatccaagaatcaggacgagtaacagtctcaacaaaaagaagtcaacaaacgataatccctaatatcggatctagaccgtgggttaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcaga

SEQ ID NO: 24 - DNA, 1061 bp, Influenza A virus
agcaaaagcaggggatatttctgtcaatcatgaagacaaccattattttgatactactgacccattgggtctacagtcaaaacccaaccagtggaaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtcttccagtatgagaattgggacctcttcatagaaagaagcagcgctttcagcaattgctacccatatgacatccctgactatgcatcgctccggtccattgtagcatcctcaggaacattggaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaaaagcagacagaattgtacatccaagaatcaggacgagtaacagtctcaacaaaaagaagtcaacaaacgataatccctaatatcggatctagaccgtgggtcaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcaga

SEQ ID NO: 25 - DNA, 1061 bp, Influenza A virus
agcaaaagcaggggatatttctgtcaatcatgaagacaaccattattttgatactactgacccattgggtctacagtcaaaacccaaccagtggaaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatataaagttctagatggaagaaattgcacattaatagatgcaatgctaggagacccccactgtgatgtcttccagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaacagcagacagaattgtacatccaagaatcaggacgagtaacagtctcaacaaaaagaagtcaacaaacgatagtccctaatatcggatctagaccgtgggttaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatataccagaaaagcaaatcaga

SEQ ID NO: 26 - DNA, 1763 bp, Influenza A virus
agcaaaagcaggggatactttctgtcaatcatgaagacaaccattattttgatactactgacccattgggtctacagtcaaaacccaaccagtggcaacaacacagccacactatgtctgggacaccatgcagtagcaaatggaacattggtaaaaacaataactgatgaccaaattgaggtgacaaatgctactgaattagttcagagcacttcaatagggaaaatatgcaacaacccatatagggttctagatggaagaaactgcacattaatagatgcaatgctaggagatccccactgtgatgtttttcagtatgagaattgggacctcttcatagaaagaagcagcgctttcagcaattgctacccatatgacatccctgactatgcatcgctccggtctattgtggcatcttcaggaacattagaattcacagcagagggattcacatggacaggtgtcactcaaaacggaggaagtggagcctgcagaagggggtcagccgatagtttctttagccgactgaattggctaacaaaatctggaaattcttaccccacattgaatgtaacaatgcctaacaataacaatttcgataaactatacatctgggggatccatcacccgagcacaaacaatgagcagacaaaattgtatatccaagaatcagggcgagtaacagtctcaacaaaaagaagtcaacaaacaataatccccaacatcggatctagaccgtgggtcaggggtcaatcaggcaggataagcatatattggaccattgtgaaacctggagatatcctaatgataaacagtaatggcaacttagttgcaccgcggggatattttaaaatgcgaacagggaaaagctctgtaatgagatcagatgcacccatagacacttgtgtgtccgagtgtattacaccaaatggaagcatccccaacgacaaaccatttcaaaatgtgaacaaagttacatatggaaaatgccccaagtatatcaagcagaatactttgaagctggccactgggatgaggaatgtaccagaaaagcaaatcagaggaatctttggagcaatagcgggattcatagaaaacggctgggaaggaatggttgatgggtggtatggattccgatatcagaattcggaaggaacaggacaagctgcagatctaaagagcactcaagcagccatcgaccagatcaatggaaaattgaacagagtgattgaaaggaccaatgagaaattccatcaaatagagaaggaattctcagaagtagaagggagaatccaggacttggagaagtatgtagaagacaccaaaatagacctatggtcctacaatgcagagttactggtggctctagaaaatcaacatacgattgacttaacagatgcagaaatgaataaattattcgagaagactaggcgccagttaagagaaaacgcggaagacatggggggtggatgtttcaagatttatcacaaatgtgataatgcatgcattggatcaataagaaatgggacatatgaccattacatatacagagatgaagcattaaacaaccgatttcaaattaaaggtgttgaattgaaatcaggctacaaagattggatactgtggatttcattcgccatatcatgcttcttaatttgcgttgttctattgggtttcatcatgtgggcttgccaaaaaggcaacatcagatgcaacatttgcatttgagtaaactgataattaaaaacacccttgtttctact

SEQ ID NO: 27 - DNA, 1027 bp, Influenza A virus
agcaaaagcaggtagatatttaaagatgagtcttctaaccgaggtcgaaacgtacgttctctctattgtaccatcaggccccctcaaagccgagatcgcgcagagacttgaagatgtctttgcagggaagaacaccgatcttgaggcactcatggaatggctaaagacaagaccaatcctgtcacctctgactaaagggattttaggatttgtgttcacgctcaccgtgcccagtgagcgaggactgcaacgtagacgctttgtccaaaatgcccttagtggaaacggagatccaaataacatggacagagcagtaaaactgtacaagaagcttaaaagagaaataacattccatggggcaaaagaggtggcactcagctattccactggtgcactagccagctgcatgggactcatatacaacagaatggggactgtgacaaccgaagtggcatttggcctggtatgcgccacatgtgaacagattgctgattcccagcatcgatctcacaggcagatggtgacaacaaccaacccactaatcagacatgaaaacagaatggtactagccagtaccacagctaaaaccatggagcaggtggcagggtcgagtgagcaggcagcagaggccatggaggttgctagtaaggccaggcagatggtgcaggcaatgaggaccattgggacccaccctagctccagtgccggtttgaaagatgatcttcttgaaaatttgcaggcctaccagaaacggatgggagtgcaaatgcagcggttcaagtgatcctctcgttattgcagcaagtatcattgggatcttgcacttgatattgtggattcttgatcgccttttcttcaaattcatttatcgtctccttaaatacggtttgaaaagagggccttctacggaaggagtacctgagtctatgagggaagaatatcggcaggaacagcagaatgctgtggatgttgacgatggtcattttgtcaacatagagctggagtaaaaaactaccttgtttctact

SEQ ID NO: 28 - DNA, 890 bp, Influenza A virus
agcaaaagcagggtgacaaaaacataatggattccaacactgtgtcaagctttcaggtagactgttttctttggcatgtccgcaaacgatttgcagaccaagaactgggtgatgccccattccttgaccggcttcgccgagaccagaagtccctaaaaggaagaggcagcactcttggtctggacatcgaaacagccactcgtgcaggaaagcagatagtggagcggattctggaagaggagtcagatgaggcacttaaaatgaccattgcctctgttcctgcttcacgctacttaactgacatgactcttgatgagatgtcaagagactggttcatgctcatgcccaagcagaaagtaacaggctccctatgtataaggatggaccaggcaatcatggataagaacatcatactaaaagcaaactttagtgtgattttcgaaaggctggagacactaatactacttagagctttcaccgaagaaggagcagtcgttggcgaaatttcaccattgccttctcttccaggacatactaatgaggatgtcaaaaatgcaattggggtcctcatcggaggacttaaatggaatgataacacagttagaatctctgaaactctacagagattcgcttggagaagcagtcatgagaatgggagaccttcattccctccaaagcagaaacgaaaaatggcgagaacaattgagtcagaagtttgaagaaataaggtggttgattgaagaagtgcgacatagattgaaaaatacagaaaatagttttgaacaaataacatttatgcaagccttacaactattgcttgaagtagaacaagagataagaactttctcgtttcagcttatttaatgataaaaaacacccttgtttctact

SEQ ID NO: 29 - DNA, 982 bp, Influenza A virus
atgagtcttctgaccgaggtcgaaacgtacgttctctctatcgtaccatcaggccccctcaaagccgagatcgcgcagagacttgaagatgtctttgcagggaagaacaccgatcttgaggcactcatggaatggctaaagacaagaccaatcctgtcacctctgactaaagggattttaggattcgtattcacgctcaccgtgcccagtgagcgaggactgcagcgtagacgctttgtccaaaatgcccttagtggaaacggagatccaaacaacatggacagagcagtaaaactgtacaggaaacttaaaagagaaataacattccatggggcaaaagaggtggcactcagctattccactggtgcactagccagctgcatgggactcatatacaacagaatgggaactgtgacaaccgaagtggcatttggcctagtatgcgccacatgtgaacagattgctgattcccagcatcgatctcacaggcagatggtgacaacaaccaacccattaatcagacatgaaaacagaatggtattagccagtaccacggctaaagccatggagcagatggcagggtcgagtgagcaggcagcagaggccatggaggttgctagtaaggctaggcagatggtacaggcaatgaggaccattgggacccaccctagctccagtgccggtttgaaaaatgatctccttgaaaatttgcaggcctaccagaaacggatgggagtgcaaatgcagcgattcaagtgatcctctcgttattgcagcaagtatcattgggatcttgcacttgatattgtggattcttgatcgccttttcttcaaattcatttatcgtcgccttaaatacgggttgaaaagagggccttctacggaaggagtacctgagtctatgagggaagaatatcggcaggaacagcagaatgctgtggatgttgacgatggtcattttgtcaacatagagctggagtaa

SEQ ID NO: 30 - DNA, 982 bp, Influenza A virus
atgagtcttctaaccgaggtcgaaacgtacgttctctctatcgtaccatcaggccccctcaaagccgagatcgcgcagagacttgaagatgtctttgcagggaagaacaccgatcttgaggcactcatggaatggctaaagacaagaccaatcctgtcacctctgactaaagggattttaggatttgtgttcacgctcaccgtgcccagtgagcgaggactgcagcgtagacgctttgtccaaaatgcccttagtggaaacggagatccaaacaacatggacagagcagtaaaactgtacaggaagcttaaaagagaaataacattccatggggcaaaagaggtggcactcagctattccactggtgcactagccagctgcatgggactcatatacaacagaatggggactgtgacaaccgaagtggcatttggcctggtatgcgccacatgtgaacagattgctgattcccagcatcgatctcacaggcagatggtgacaacaaccaacccactaatcagacatgaaaacagaatggtactagccagtaccacagctaaagccatggaacagatggcagggtcgagtgagcaggcagcagaggccatggaggttgctagtaaggccaggcagatggtacaggcaatgaggaccattgggacccaccctagctccagtgccggtttgaaagatgatcttcttgaaaatttgcaggcctaccagaaacggatgggagtgcaaatgcagcgattcaagtgaccctctcgttattgcagcaagtatcattgggatcttgcacttgatattgtggattcttgatcgccttttcttcaaattcatttatcgtcgccttaaatacggtttgaaaagagggccttctacggaaggagtacctgagtctatgagggaagaatatcggcaggaacagcagaatgctgtggatgttgacgatggtcattttgtcaacatagagctggagtaa

SEQ ID NO: 31 - DNA, 838 bp, Influenza A virus
atggattccaacactgtgtcaagctttcaggtagactgttttctttggcatgtccgcaaacgattcgcagaccaagaactgggtgatgccccattccttgaccggcttcgccgagaccagaagtccctaaaaggaagaggtagcactcttggtctggacatcgaaacagccactcgtgcaggaaagcagatagtggagcagattctggaagaggaatcagatgaggcacttaaaatgaccattgcctctgttcctgcttcacgctacttaactgacatgactcttgatgagatgtcaagagactggttcatgctcatgcccaagcagaaagtaacaggctccctatgtataagaatggaccaggcaatcatggataagaacatcatacttaaagcaaactttagtgtgattttcgaaaggctggaaacactaatactacttagagccttcaccgaagaaggagcagtcgttggcgaaatttcaccattgccttctcttccaggacatactaatgaggatgtcaaaaatgcaattggggtcctcatcggaggacttaaatggaatgataatacggttagaatctctgaaactctacagagattcgcttggagaagcagtcatgagaatgggagaccttcattccctccaaagcagaaacgaaaaatggagagaacaattgagccagaagtttgaagaaataagatggttgattgaagaagtgcgacatagattgaaaaatacagaaaatagttttgaacaaataacatttatgcaagccttacaactattgcttgaagtagaacaagagataagaactttctcgtttcagcttatttaa

SEQ ID NO: 32 - DNA, 838 bp, Influenza A virus
atggattccaacactgtgtcaagctttcaggtagactgttttctttggcatgtccgcaaacgatttgcagaccaagaactgggtgatgccccattccttgaccggcttcgccgagaccagaagtccctaaaaggaagaggcagcactcttggtctggacatcgaaacagccactcgtgcaggaaagcagatagtggagcagattctggaagaggaatcagatgaggcacttaaaatgaccattgcctctgttcctgcttcacgctacttaactgacatgactcttgatgagatgtcaagagactggttcatgctcatgcccaagcagaaagtaacaggctccctatgtataaggatggaccaggcaatcatggataagaacatcatactaaaagcaaactttagtgtgattttcgaaaggctggagacactaatactacttagagccttcaccgaagaaggagcagtcgttggcgaaatttcaccattgccttctcttccaggacatactaatgaggatgtcaaaaatgcaattggggtcctcatcggaggacttaaatggaatgataatacagttagagtctctgaaactctacagagattcgcttggagaagcagtcatgagaatgggagaccttcattccctccaaagcagaaacgaaaaatggcgagaacaattgagccagaagtttgaagaaataagatggttgattgaagaagtgcgacatagattgaaaaatacagaaaatagttttgaacaaataacatttatgcaagccttacaactattgcttgaagtagaacaagagataagaactttctcgtttcagcttatttaa

SEQ ID NO: 33 - DNA, 1565 bp, Influenza A virus
agcaaaagcagggtagataatcactcactgagtgacatcaaagtcatggcgtctcaaggcaccaaacgatcttatgagcagatggaaactggtggggaacgccagaatgcaactgaaatcagagcatctgtcggaaggatggtgggaggaatcggccggttctatgttcagatgtgtactgagcttaaactcaacgaccatgaagggcggctgattcagaacagcataacaatagaaaggatggtactttcggcattcgacgaaagaagaaacaagtacctcgaggagcatcccagtgctgggaaagaccccaagaaaacgggaggcccgatatacagaaggaaagatgggaaatggatgagagaactcatcctccatgataaagaagaaatcatgaggatctggcgtcaggccaacaatggtgaagacgctactgctggtcttactcatatgatgatctggcactccaatctcaatgacaccacataccaaagaacaagggctcttgttcgggctgggatggatcccagaatgtgctctctgatgcaaggatcaaccctcccacggagatctggagctgccggtgctgcagtaaaaggtgttggaacaatggtaatggaactcatcaggatgatcaaacgcgggataaatgatcgaaatttctggagaggtgaaaatggtcgaagaaccagaattgcttatgaaagaatgtgcaatatcctcaaagggaaattccaaacagcagcacaacgggcaatgatggaccaagtgagggagggccgcaatcctggaaatgctgagattgaggatctcattttcttggcacgatcagcactcattttgagaggatcagtagcccataaatcatgcctacctgcctgtgtttatggccttgcagtagccagtgggtatgactttgagaaggaaggatactctctggttggaattgatcctttcaaactactccagaacagccaaattttcagtctaatcagaccgaaagaaaatccagcacacaagagccagttggtgtggatggcatgccattctgcagcatttgaggacctgagagttttgaatttcattagaggaaccaaagtaatcccaagaggacagttagcaaccagaggagtgcaaattgcttcaaatgaaaacatggagacaatagattctagcacactcgaactgaggagcagatattgggcaataaggaccaggagtggagggaacaccagtcaacagagagcatctgcaggacagataagtgtgcaacccactttctcagtgcagagaaatcttccctttgaaagagcaaccattatggctgcattcactgggaacactgagcggaggacttccgacatgagaacggaaatcataaggatgatggaaaatgccagatcagaagatgtgtctttccaggggcggggagtcttcgagctctcggacgaaaaggcaacgaacccgatcgtgccttcctttgacatgagcaatgaagggtcttatttcttcggagacaatgctgaggagtttgacagttaaagaaaaatacccttgtttctact

SEQ ID NO: 34 - DNA, 2341 bp, Influenza A virus
agcaaaagcaggtcaaatatattcaatatggagagaataaaagaactgagagatctaatgtcacagtcccgcacccgcgagatactaacaaaaactactgtggaccatatggccataatcaagaaatacacatcaggaagacaagagaagaaccccgcacttaggatgaagtggatgatggcaatgaaatacccaattacagcagataagaggataatggaaatgattcctgagagaaatgaacaggggcaaaccctttggagcaaaacgaacgatgctggctcagaccgcgtaatggtatcacctctggcagtgacatggtggaataggaatggaccaacaacgagcacaattcattatccaaaagtctacaaaacttattttgaaaaagttgaaaggttaaaacacggaacctttggccccgttcattttaggaatcaagtcaagataagacggagagttgacgtaaaccctggtcacgcggacctcagtgccaaagaagcacaagatgtgatcatggaagttgttttcccaaatgaagtgggagccagaattctaacatcggaatcacaactaacaataaccaaagagaaaaaagaagaacttcaggactgcaaaattgcccccttgatggtagcatacatgctagaaagagagttggtccgaaaaacaaggttcctcccagtggctggcggaacaagcagtgtatacattgaggtgttgcatctgactcagggaacgtgctgggaacaaatgtacaccccaggaggagaagttagaaacgatgacattgatcaaagtttaattattgctgcccggaacatagtgaaaagagcgacagtatcagcagatccactagcatccctgctggagatgtgccacagtacacagattggtggaataaggatggtagacatccttaagcagaatccaacagaggaacaagctgtggatatatgcaaagcagcaatggggttaagaattagctcatcattcagctttggtggattcacctttaagagaacaagtggatcatcagtcaagagagaagaagaaatgcttacgggcaaccttcaaacattgaaaataagagtgcatgagggctatgaagaattcacaatggtcggaagaagagcaacagccattctcagaaagacaaccagaagattgattcaattgatagtaagtgggagagatgaacagtcaattgctgaagcaataattgtagccatggtgttttcgcaagaagattgcatgataaaagcagttcgaggcgatttgaacttcgttaatagagcaaatcagcgcttgaaccccatgcatcaactcttgaggcatttccaaaaggatgcaaaagtgcttttccagaattgggggattgaacccatcgacaatgtgatgggaatgatcggaatattgcccgacatgaccccaagcaccgagatgtcattgagaggagtgagagtcagcaaaatgggagtggatgagtactccagcactgagagagtggtggtgagcattgaccgttttttaagagttcgggatcaaaggggaaacatactactgtcccctgaagaggtcagtgaaacacaaggaacggaaaagctgacaataatttattcatcatcaatgatgtgggagattaatggtcccgagtcagtgttggtcaatacttatcaatggatcatcagaaactgggaaattgtgaaaattcaatggtcacaggatcccacaatgttatacaataagatagaatttgagccattccagtccctggtccctagggccaccagaagccaatacagcggtttcgtaaggaccctgtttcagcaaatgcgagatgtacttggaacatttgacactgctcaaataataaaactcctcccttttgccgctgctcctccggaacagagtagaatgcagttctcttctttgactgttaatgtaagaggatcgggaatgaggatacttgtaagaggcaattccccagtgttcaactacaacaaagccactaagaggctcacagtcctcggaaaggatgcaggtgcgcttactgaagacccagatgaaggtacggctggagtagaatctgctgttctgagagggtttctcatcttaggtaaagaaaacaagagatatggcccagcactaagcatcaatgaactgagcaaacttacaaaaggggagaaagctaatgtgctaattgggcaaggggacgtggtgttggtaatgaaacggaaacgtgactctagcatacttactgacagtcagacagcgaccaaaaggattcggatggccatcaattagtgttgaattgtttaaaaacgaccttgtttctact

SEQ ID NO: 35 - DNA, 1762 bp, Influenza A virus
  modified_base (313): a, c, g, t, unknown or other
agcaaaagcaggggatatttctgtcaatcatgaagacaaccattgttttgatactactgacccattgggtctacagtcaaaacccaaccagtggcaacaacacagccacattatgtctgggacaccatgcagtagcaaatggaacactggtaaaaacaataactgatgaccagattgaggtgacaaatgctactgaattagttcagagcatttcaatagggaaaatatgcaacaactcatatagggttctagatggaagaaattgcacattaatagatgcaatgctaggagacccccactgtgatgtttttcngtatgagaattgggacctcttcatagaaagaagcagcgctttcagcaattgctacccatatgacatccctgactatgcatcgctccggtctattgtggcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccacattgaatgtgacaatgcctaacaataacaatttcgataaactatacatctgggggattcatcacccgagctcaaacaatgagcagacaaaattgtatatccaagaatcaggacgagtaacagtctcaacaaaaagaagtcaacaaacaataatccccaacatcggatctagaccgtgggtcaggggtcaatcaggcaggataagcatatattggaccattgtgaaacctggagatatcctaatgataaacagtaatggcaacttagttgcaccgcggggatatttcaaattgagaacagggaaaagctctgtaatgagatcagatgcacccatagacacttgtgtgtctgaatgtattacaccaaatggaagcatccccaacgacaaaccattccaaaatgtgaacaaagttacatatggaaaatgccccaagtatatcaggcaaaacactttgaagctggccactgggatgaggaatgtaccagaaaagcaaatcagaggaatctttggagcaatagcgggattcatagaaaacggctgggaaggaatggttgatgggtggtatggattccgatatcaaaattcggaaggaacaggacaagctggagatctaaagagcactcaagcagccatcgaccagatcaatggaaaattaaacagagtgattgaaaggaccaatgagaaattccatcaaatagagaaggaattctcagaagtagaagggagaatccaggacttggagaagtatgtagaagacaccaaaatagacctatggtcctacaatgcagaattgctggtggctctagaaaatcaacatacaattgacttaacagatgcagaaatgaataaattattcgagaagactaggcgccagttaagagaaaacgcggaagacatgggaggtggatgtttcaggatttaccacaaatgtgataatgcatgcattggatcaataagaaatgggacatatgaccattacatatacagagatgaagcattaaacaaccgatttcaaattaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 36 - DNA, 1762 bp, Influenza A virus
  modified_base (313): a, c, g, t, unknown or other
agcaaaagcaggggatatttctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtttttcngtatgagaattgggacctcttcatagaaagaagcagcgcttccagcaattgctacccatatgacatccctgactatgcatcgctccggtctattgtggcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccacattgaatgtgacaatgcctaacaataacaatttcgataagctatacatctgggggatccatcacccgagctcaaacaatgagcagacaaaattgtatatccaagaatcaggacgagtaacagtctcaacaaaaagaagtcaacaaacaataatccccaacatcggatctagaccgtgggtcaggggtcaatcaggcaggataagcatatattggaccattgtgaaacctggagatatcctaataataaacagtaatggcaacttagttgcaccgcggggatatttcaaattgcgaacagggaaaagctctgtaatgagatcagatgcacccatagacacttgtgtgtctgaatgtattacaccaaatggaagcatccccaacgacaaaccatttcaaaatgtgaacaaagttacatatggaaaatgccccaagtatatcaggcaaaacactttgaagctggccactgggatgaggaatgtaccagaaaagcaaatcagaggaatctttggagcaatagcgggattcatagaaaacggctgggaaggaatggttgatgggtggtatggattccgatatcaaaactcggaaggaacaggacaagctggagatctaaagagcactcaagcagccatcgaccagatcaatggaaaattgaacagagtgattgaaaggaccaatgagaaattccatcaaatagagaaggaattctcagaagtagaagggagaatccaggacttggagaagtatgtagaagacaccaaaatagacctatggtcctacaatgcagagttgctggtggctctagaaaatcaacatacaattgacttaacagatgcagaaatgaataaactattcgagaagactaggcgccagttaagagaaaacgcggaagacatgggaggtggatgtttcaagatttatcacaaatgtgataatgcatgcattggatcaataagaaatgggacatatgaccattacatatacagagatgaagcattaaacaaccgatttcaaattaaaggtgtagagctgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 37 - DNA, 693 bp, Influenza A virus
atggattccaacactgtgtcaagctttcaggtagactgttttctttggcatgtccgcaaacgatttgcagaccaagaactgggtgatgccccattccttgaccggcttcgccgagaccagaagtccctaaaaggaagaggtagcactcttggtctggacatcgaaacagccactcgtgcaggaaagcagatagtggagcagattctggaagaggaatcagatgaggcatttaaaatgaccattgcctctgttcctgcttcacgctacttaactgacatgactcttgatgagatgtcaagagactggttcatgctcatgcccaagcagaaagtaacaggctccctatgtataagaatggaccaggcaatcatggataagaacatcatacttaaagcaaactttagtgtgattttcgaaaggctggagacactaatactactcagggccttcaccgaagaaggagcagtcgttggcgaaatttcaccattgccttctcttccaggacatactaatgaggatgtcaaaaatgcaattggggtcctcatcggaggacttaaatggaatgataatacggttagagtctctgaaactctacagagattcgcttggagaagcagtcatgagaatgggagaccttcattccctccaaagcagaaacgaaaaatggagagaacaattgagtcagaagtttga

SEQ ID NO: 38 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagtccagagcatttcaatagggaaaatatgcaacaactcatatagggttctagatggaagaaattgcacattaatagatgcaatgctaggagacccccattgtgatgattttcagtatgagaattgggacctcttcatagaaagaagcagcgctttcagcaattgctacccatatgacatccctgactatgcatcgctccggtccattgtagcatcctcaggaacattagaattcacagcagaggggttcacatggacaggtgtcactcaaaacggaggaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattatatatccaagaatcaggacgagtaacagtctcaacagaaagaagtcaacaaacagtaatccctaacatcggatctaggccgtgggtcaggggtcaatcaggcaggataagcatatactggaccattgtaaaacctggagatattctaatgataaacagtaatggcaacttagttgcaccgcggggatattttaaattgagaacagggaaaagctctgtaatgagatcagatgcactcatagacacttgtgtgtctgaatgtattacaccaaatggaagcatccccaacgacaaaccatttcaaaatgtgaacaaaattacatatggaaaatgccccaagtatatcaggcaaaacactttaaagctggccactgggatgaggaatgtaccagaaaagcaaatcaga

SEQ ID NO: 39 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtctttcagtatgagaattgggacctcttcatagaaagaagcagcgctttcagcaattgctacccatatgacatccctgactatgcatcgctccggtccattgtagcatcctcaggaacattggaattcacagcagagggattcacatggacaggtgtcactcaaaacggaagaagtggagcctgcaaaaggggatcagccgatagtttctttagccgactgaattggctaacaaaatctggaaactcttaccccacattgaatgtgacaatgcctaacaataaaaatttcgacaaactatacatctgggggattcatcacccgagctcaaaccaacagcagacagaattgtacatccaagaatcaggacgagtaacagtctcaacaaaaagaagtcaacaaacgataatccctaatatcggatctagaccatgggtcaggggtcaatcaggcaggataagcatatactggaccattgtaaaacctggagatatcctaatgataaacagtaatggcaacttagttgcaccgcggggatattttaaattgaaaacagggaaaagctctgtaatgagatcagatgcacccatagacatttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcaga

SEQ ID NO: 40 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtttttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattgtatatccaagaatcaggacgagtaacagtctcaacagaaagaagtcaacaaacagtaatccctaacatcggatctagaccgtgggtcaggggtcaatcaggcaggataagcatatactggaccattgtaaaacctggagatattctaatgataaacagtaatggcaacttagttgcaccgcggggatattttaaattgagaacagggaaaagctctgtaatgagatcagatgcactcataggcacttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcaga

SEQ ID NO: 41 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccattgtgatgattttcagtatgagaattgggacctcttcatagaaagaagcagtgctttcagcaattgctacccatatggcatccctgactatgcatcgctccggtccattgtagcatcctcaggaacattagaattcacagcagaggggttcacatggacaggtgtcactcaaaacggaagaagtggagcctgcaaaaggggatcagccgatagtttctttagccgactcaattggctaacaaaatctggaaattcttaccccatattgaatgtgacaatgcctaacaataaaaatttcgataaactatacatctgggggattcatcacccgagctcaaacaaagagcagacaaaattatatatccaagaatcaggacgagtaacagtctcaacagaaagaagtcaacaaacagtaatccctaacatcggatctaggccgtgggtcaggggtcaatcaggcaggataagcatatactggaccattgtaaaacctggagatattctaatgataaacaataatggcaacttagttgcaccgcggggatattttaaattgagaacagggaaaagctctgtaatgagatcagatgcactcatagacacttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatataccagaaaagcaaatcaga

SEQ ID NO: 42 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtttttcagtatgagaattgggacctcttcatagaaagaagcagcgctttcagcaattgctacccatatgacatctctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattgtatatccaagaatcaggacgagtaacagtctcaacagaaagaagtcaacaaacagtaatccctaacatcggatctagaccgtgggtcaggggtcaatcaggcaggataagcatatactggaccattgtaaaacctggagatattctaatgataaacagtaatggcaacttagttgcaccgcggggatattttaaattgagaacagggaaaagctctgtaatgagatcagatgcacccataggcacttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcaga

SEQ ID NO: 43 - DNA, 1032 bp, Influenza A virus
atgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagagttctagatggaagaaattgcacattaatagatgcaatgctaggagacccccactgtgatgtctttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattgtacatccaagaatcaggacgagtaacagtctcaacaaaaagaagtcaacaaacaataatccctaacatcggatctagaccgtgggtcaggggtcaatcaggcaggataagcatatactggaccattgtaaaacctggagatatcctaatgataaacagtaatggcaacttagttgcaccgcggggatattttaaattgagaacagggaaaagctctgtaatgagatcagatgcacccatagacatttgtgtgtctgaatgtattacaccaaatggaagcatccccagcgacaaaccatttcaaaatgtaaacaaagttacatatggaaaatgccccaagtatatcaggcaaaacactttaaagctggccactgggatgaggaatgtaccagaaaagcaaatcaga

SEQ ID NO: 44 - DNA, 1742 bp, Influenza A virus
ctgtcaatcatgaagacaaccattattttgatactactgacccattgggtctacagtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtttttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattgtatatccaagaatcaggacgagtaacagtctcaacagaaagaagtcaacaaacagtaatccctaacatcggatctagaccgtgggtcaggggtcaatcaggcaggataagcatatactggaccattgtaaaacctggagatattctaacgataaacagtaatggcaacttagttgcaccgcggggatattttaaattgagaacagggaaaagctctgtaatgagatcagatgcacccatagacacttgtgtgtctgaatgtattacaccaaatggaagcatccccaacgacaaaccatttcaaaatgtgaacaaagttacatatggaaaatgccccaagtatatcaggcaaaacactttaaagctggccaccgggatgaggaatgtaccagaaaagcaaatcagaggaatctttggagcaatagcgggattcatagaaaacggctgggaaggaatggttgatgggtggtatggattccgatatcaaaactcggaaggaacaggacaagctgcagatctaaagagcactcaagcagccatcgaccagatcaatggaaaattaaacagagtgattgaaaggaccaatgagaaattccatcaaatagagaaggaattctcagaagtagaagggagaatccaggatttggagaagtatgtagaagacaccaaaatagacctatggtcctacaatgcagaattgctggtggctctagaaaatcaacatacaattgacttaacagatgcagaaatgaataaattattcgagaagactaggcgccagttaagagaaaacgcggaagacatgggaggtggatgtttcaagatttaccacaaatgtgataatgcatgcattggatcaataagaaatgggacatatgaccattacatatacagagatgaagcattaaacaaccgatttcaaatcaaaggtgttgagttgaaatcaggctacaaagattggatactgtggatttcattcgccatatcatgcttcttaatttgcgttgttctattgggtttcattatgtgggcttgccaaaaaggcaacatcagatgcaacatttgcatttgagtaaactgatagttaaaaacacccttgtttctact

SEQ ID NO: 45 - DNA, 1761 bp, Influenza A virus
  modified_base (1)..(19): a, c, g, t, unknown or other
nnnnnnnnnnnnnnnnnnnaaatgaacactcagattctaatattagccatttcggcattcctctgtgtacgtgcagataaaatctgcctaggacatcatgctgtgtctaatggaaccaaagtagacacccttactgaaaagggaatagaagtcgtcaatgcaacagaaacagttgaacaaaaaaacatccccaagatctgctcaaaagggaaacagactattgaccttggtcaatgtggattactagggaccactattggtcccccccaatgcgaccaatttcttgaattctctgctaatttaataattgagagaagagaaggtgatgatatttgttatccaggcaaatttgacaatgaagaaacattgagacaaatactcagaaaatccggaggaattaaaaaggagaatatgggattcacatataccggagtgagaaccaatggagagactagcgcctgtagaaggtcaagatcttccttttatgcagaaatgaaatggctcctatctaacacagacaatggggtattcccacaaatgacaaaatcctacaagaacactaagaaggagccagctctgataatctggggaatccaccactcaggatcaactgctgaacagactagattgtatggaagtggaaacaagttgataacagtttggagttccaaataccaacaatcttttgccccaaaccctggaccaaggccgcaaatgaatggccaatcaggaagaattgacttttactggctgatgttagatcccaatgatactgttaatttcagttttaatggggcctttatagcacctgaccgcgccagttttctaagaggtaaatctctaggaattcagagtgacgcacaacttgacaacaattgtgaaggtgaatgttatcatattggaggtaccataattagcaacttgccctttcaaaacattaatagcagagcaattgggaaatgccccagatacgtaaagcaaaaaagcttaatgctagcaaccggaatgaaaaatgttcctgaaaattctacacacaaacagttaactcatcacatgcgcaaaaaaagaggtttatttggtgcaatagcaggatttattgaaaatggatgggaaggattaatagatggatggtatggatacagacatcagaatgcacaaggagaaggaactgctgcagactacaaaagtacacaatctgctgtcaatcaaataaccgggaaattaaacagactaatagaaaaaaccaaccagcaatttgaactaatagataatgaattcaatgaaatagaaaagcaaattggcaatgttattaactggactagagattctatcatcgaaatatggtcatataatgcagaattcctcgtggcagtggagaatcaacacactattgatttaactgattcagagatgaacaaattatatgaaaaggtaagaagacagctgagagaaaatgctgaggaagatggtaatggctgttttgaaatatttcaccaatgtgacaatgattgcatggccagcattagaaacaatacatatgatcataaaaaatacagaaaggaggcaatacaaaacagaattcagattgatgcagtaaagttgagcagcggttacaaagaaataatactttggtttagcttcggggcatcatgtttcttatttcttgccattgcaatggttcttgctttcatatgcataaaaaatggaaacatgcggtgcactatttgtatataagtttgaaaaaacacccttgtttctann

SEQ ID NO: 46 - DNA, 1096 bp, Influenza A virus
agtcaaaacccaaccagtggcaacaacacagccacattatgtctgggacaccatgcagtagcaaatggaacattggtaaaaacaataactgatgaccaaattgaggtgacaaatgctactgaattagttcagagcatttcaatagggaaaatatgcaacaactcatatagggttctagatggaagaaattgcacattaatagatgcaatgctaggagacccccactgtgatgtttttcagtatgagaattgggacctcttcatagaaagaagcagcgctttcagcaattgctacccatatgacatccctgactatgcatcgctccggtccattgtagcatcctcaggaacattagaattcacagcagagggattcacatggacaggtgtcactcaaaacggaagaagtggagcctgcaaaaggggatcagccgatagtttctttagccgactgaattggctaacaaaatctggaaattcttaccccatattgaatgtgacaatgcctaacaataaaaatttcgataaactatacatctgggggattcatcacccgagctcaaacaaagagcagacaaaattgtatatccaagaatcaggacgagtaacagtctcaacagaaagaagtcaacaaacagtaatccctaacatcggatctagaccgtgggtcaggggtcaatcaggcaggataagcatatactggaccattgtaaaacctggagatattctaatgataaacagtaatggcaacttagttgctccgcggggatattttaaattgagaacagggaaaagctctgtaatgagatcagatgcacccatagacacttgtgtgtctgaatgtattacaccaaatggaagcatccccaacgacaaaccatttcaaaatgtgaacaaagttacatatggaaaatgccccaagtatatcaggcaaaacactttaaagctggccactgggatgaggaatgtaccagaaaagcaaatcagaggaatctttggagcaatagagggattcatagaaaacggctgggaaggaatggttgatgggtggtatggattccgatatcaaaactcggaaggaacaggacaagctgcag

SEQ ID NO: 47 - DNA, 1086 bp, Influenza A virus
ctgtcaatcatgaagacaaccattattttgatactactgacccattgggtctacagtcaaaacccaaccagtggcaacaacacagccacactatgtctgggacaccatgcagtagcaaatggaacattggtaaaaacaataactgatgaccaaattgaggtgacaaatgccactgaattagttcagagcacttcaatagggaaaatatgcaacaacccatatagggttctagatggaagaaactgcacattaatagatgcaatgctaggagatccccactgtgatgtttttcagtatgagaattgggacctcttcatagaaagaagcagcgctttcagcaattgctacccatatgacatccctgactatgcatcgctccggtctattgtggcatcttcaggaacattagaattcacagcagagggattcacatggacaggtgtcactcaaaacggaagaagtggcgcctgcagaaggggatcagccgatagtttctttagccgactgaattggctaacaaaatctggagattcttaccccacattgaatgtgacaatgcctaacaataacaatttcgataaactatacatctgggggatccatcacccgagcacaaacaatgagcagacaaaattgtatgtccaagaatcagggcgagtaacagtctcaacaaaaagaagtcaacaaacaataatccccaacatcggatctagaccgtgggtcaggggtcaaccaggcaggataagcatatattggaccattgtgaaacctggagatatcctaatgataaacagtaatggcaacttagttgcaccgcggggatatttcaaaatgcgaacaggaaaaagctctataatgagatcagatgcacccatagacacttgtgtgtccgagtgtattacaccaaatggaagcatccccaacgacaaaccatttcaaaatgtgaacaaagttacatatgggaaatgccccaagtatatcaagcagaatactttgaagctggccactgggatgaggaatgtaccagaaaagcaaatcagaggaatctttggagcaatagcgggattcatagaaaatggctgggag

SEQ ID NO: 48 - DNA, 982 bp, Influenza A virus
atgagtcttctgaccgaggtcgaaacgtacgttctctctatcgtaccatcaggccccctcaaagccgagatcgcgcagagacttgaagatgtctttgcaggaaagaacaccgatcttgaggcactcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggagatccaaacaacatggacagagcagtaaaactgtacaggaagcttaaaagggaaataacattccatggggcaaaagaggtggcactcagctattccactggtgcactagccagctgcatgggactcatatacaacagaatggggactgtgacaaccgaagtggcatttggcctggtatgcgccacatgtgaacagattgctgattcccagcaccgatctcacagacagatggtgacaacaaccaacccactaatcagacacgagaacagaatggtactagccagtaccacagctaaagccatggagcagatggcagggtcgagtgagcaggcagcagaggccatggaggttgctagtcaggccaggcagatggtgcaggcaatgagaaccattgggacccaccctagctccagtgccggtttgaaaaatgatcttcttgaaaatttgcaggcctaccagaaacggatgggagtgcaaatgcagcgattcaagtgatcctctcgttattgcagcaagtatcattgggatcttgcacttgatattgtggattcttgatcgtcttttcttcaaatgcatttatcgtcgtcttaaatacggtttgaaaagagggccttctacggaaggagtacctgagtctatgagggaagaatatcggcaggaacagcagagtgctgtggatgttgacgatggtcattttgtcaacatagagctggagtaa

SEQ ID NO: 49 - DNA, 982 bp, Influenza A virus
atgagtcttctaaccgaggtcgaaacgtacgttctctctatcgtaccatcaggccccctcaaagccgagatcgcgcagagacttgaagatgtctttgcagggaagaacaccgatcttgaggcactcatggaatggctaaagacaagaccaatcctgtcacctctgactaaagggattttaggatttgtgttcacgctcaccgtgcccagtgagcgaggactgcagcgtagacgctttgtccaaaatgcccttagtggaaacggagatccaaacaacatggacagagcagtaaaactgtacaggaagcttaaaagagaaataacattccatggggcaaaagaggtggcactcagctattccactggtgcactagccagctgcatgggactcatatacaacagaatggggactgtgacaaccgaagtggcatttggcctggtatgcgccacatgtgaacagattgctgattcccagcatcgatctcacaggcagatggtgacaacaaccaacccactaatcagacatgaaaacagaatggtactagccagtaccacagctaaagccatggagcagatggcagggtcgagtgagcaggcagcagaggccatggaggttgctagtaaggccaggcagatggtacaggcaatgaggaccattgggacccaccctagctccagtgccggtttgaaagatgatcttcttgaaaatttgcaggcctaccagaaacggatgggagtgcaaatgcagcgattcaagtgatcctctcgttattgcagcaagtatcattgggatcttgcacttgatattgtggattcttgatcgccttttcttcaaattcatttatcgtcgccttaaatacggtttgaaaagagggccttctacggaaggagtacctgagtctatgagggaagaatatcggcaggaacagcagaatgctgtggatgttgacgatggtcattttgtcaacatagagctggagtaa

SEQ ID NO: 50 - DNA, 838 bp, Influenza A virus
atggattccaacactgtgtcaagctttcaggtagactgttttctttggcatgtccgcaaacgatttgcagaccaagaactgggtgatgccccattccttgaccggcttcgccgagaccagaagtccctaaaaggaagaggcagcactcttggtctggacatcgaaacagccactcatgcaggaaagcagatagtggagcgaattctggaagaggaatcagatgaggcacttaaaatgaccatagcctctgttcctacttcacgctacttaactgacatgactcttgatgagatgtcaagagactggttcatgctcatgcccaagcagaaagtaacaggctccctatgtataaggatggaccaagcaatcatggataagaacatcatactaaaagcaaactttagtgtgattttcgaaaggctggagacactaatactacttagagctttcaccgaagaaggagcagtcgttggcgaaatttcaccattgccttctcttccaggacatactaatgaggatgtcaaaaatgcaattggggtcctcatcggaggacttaaatggaatgataacacagttagagtctctgaaactctacagagattcgcttggagaagcagtcatgagaatgggagaccttcattccctccaaagcagaaacgaaaaatggcgagaacaattgagtcagaagtttgaagaaataaggtggttgattgaagaagtgagacatagattgaaaaatacagaaaatagttttgaacaaataacatttatgcaagccttacaactattgcttgaagtagaacaagagataagaactttctcgtttcagcttatttaa

SEQ ID NO: 51 - DNA, 838 bp, Influenza A virus
atggattccaacactgtgtcaagctttcaggtagactgttttctttggcatgtccgcaaacgatttgcagaccaagaactgggtgatgccccattccttgaccggcttcgccgagaccagaagtccctaaaaggaagaggcagcactcttggtctggacatcgaaacagccactcgtgcaggaaagcagatagtggagcggattctggaagaggagtcagatgaggcacttaaaatgaccattgcctctgttcctgcttcacgctacttaactgacatgactcttgatgagatgtcaagagactggttcatgctcatgcccaagcagaaagtaacaggctccctatgtataaggatggaccaggcaatcatggataagaacatcatactaaaagcaaactttagtgtgattttcgaaaggctggagacactaatactacttagagccttcaccgaagaaggagcagtcgttggcgaaatttcaccattgccttctcttccaggacatactaatgaggatgtcaaaaatgcaattggggtcctcatcggaggacttaaatggaatgataatacagttagagtctctgaaactctacagagattcgcttggagaagcagtcatgagaatgggagaccttcattccctccaaagcagaaacgaaaaatggcgagaacaattgagccagaagtttgaagaaataagatggttgattgaagaagtgcgacatagattgaaaaatacagaaaatagttttgaacaaataacatttatgcaagccttacaactattgcttgaagtagaacaagagataagaactttctcgtttcagcttatttaa